(19) United States
(12) Patent Application Publication
(10) Pub. No.: US 2021/0338299 A1
(43) Pub. Date: Nov. 4, 2021

(54) ANATOMICAL STRUCTURE MOUNTING APPARATUSES

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Mouhsin El-Chafei, Arlington, TN (US); David W. Rister, Nesbit, MS (US); Michael D. Hollandsworth, JR., Memphis, TN (US); Daniel Cook, Cordova, TN (US); Jeffrey Lee, Memphis, TN (US); Ashley A. Roakes, Memphis, TN (US)

(21) Appl. No.: 17/243,045

(22) Filed: Apr. 28, 2021

Related U.S. Application Data
(60) Provisional application No. 63/017,403, filed on Apr. 29, 2020.
(60) Provisional application No. 63/017,368, filed on Apr. 29, 2020.
(60) Provisional application No. 63/017,384, filed on Apr. 29, 2020.

(51) Int. Cl. A61B 17/88; B25J 9/10; A61B 34/10; A61B 34/20

(52) U.S. Cl. CPC A61B 17/8866; B25J 9/1035; A61B 2034/105; A61B 34/20; A61B 2034/102; A61B 34/10

(57) ABSTRACT

Apparatuses for mounting medical aid devices to anatomical structures and methods of use are disclosed. Mounting apparatuses may include femoral clamps or clamps configured to be affixed to other bone structures. Mounting apparatuses may include a pair of opposing arms configured to be compressed around a portion of a bone structure via a clamping force provided by a clamping assembly to affix the mounting apparatus to the bone structure. Clamping assemblies may include a magnetically-actuated clamping assembly, a mechanically-actuated clamping assembly, or a spring-actuated clamping assembly. A magnetically-actuated clamping assembly may generate a clamping force via alignment of magnetic fields within the clamping apparatus. A mechanically-actuated clamping assembly may generate a clamping force via a linkage-tensioning system, a rack-and-pinion system, or a lever-locking system. A spring-actuated clamping assembly may generate a clamping force via a torsion spring having arms that bias the pair of opposing arms toward the bone structure.

### CROSS-REFERENCE TO RELATED APPLICATIONS

This is a non-provisional of, and claims the benefit of the filing date of, the following pending U.S. Provisional Patent Applications Numbers: 63/017,403, filed Apr. 29, 2020, titled “Anatomical Structure Mounting Apparatus;” 63/017,368, filed Apr. 29, 2020, titled “Anatomical Structure Mounting Apparatus;” and 63/017,384, filed Apr. 29, 2020, titled “Anatomical Structure Mounting Apparatus.” Each of the aforementioned applications are incorporated by reference herein in their entirety

### FIELD OF THE DISCLOSURE

The present disclosure relates generally to apparatuses for mounting devices on or within anatomical structures, and, more particularly, to clamps configured to use one or more of a magnetically-actuated clamping assembly, a spring-actuated clamping assembly, or a mechanically-actuated clamping assembly to temporarily and rigidly couple medical aid devices to a bone structure of a patient.

### BACKGROUND OF THE DISCLOSURE

Certain medical procedures and treatments use medical aid devices that are attached to internal portions of a patient. For example, computer-assisted surgery (CAS) navigation systems, such as computer- or robotic-assisted hip and knee replacement procedures, may use tracking arrays affixed to various bone structures in conjunction with a camera or other tracking device to assist in establishing anatomical landmarks and to facilitate surgical tool navigation.

Navigated surgical approaches can require additional steps compared with traditional, non-navigated surgical workflows. Illustrative additional steps may include confirming implant placement with trackable surgical instruments and verifying anatomical tracking parameters with pointers. For example, for hip replacement procedures, an additional step may include the attachment of tracking arrays to parts of the pelvis and femur.

The attachment of tracking arrays via conventional devices can require additional materials, such as adhesives or screws, surgical steps (for example, incisions), and time to perform the overall procedure. In the case of a femur, in order for the tracking camera to detect an array as a landmark, the tracking array must be placed along the femoral shaft via a rigid connection that is also able to accept different femur shapes, sizes, and other patient variances. In a direct anterior approach for a total hip replacement, a single incision at the proximal femur is created to perform the procedure. In a navigated surgical approach, a tracking array may be attached to the distal femur away from the proximal incision, necessitating another incision at the distal femur. Such a requirement adds a significant step to the overall procedure and may possibly impede the manipulating and positioning of the femur or other anatomical structures. Additional incisions may also increase the possibility of the patient contracting an infection, and may cause additional post-operative pain.

Conventional techniques for attaching medical aid devices to bony anatomy have included permanent (semi-permanent or fastener-affixed) and temporary (or releasably-coupled) methods for attaching a mounting device configured to hold the medical aid device to a portion of the bone. Permanent methods generally include attaching the mounting device to the bone using a pin (or screw) and/or an adhesive. For example, pin-based methods have used a fixator device that is attached directly to the femur of a surgical patient via a Schanz screw that is driven into the femur at a selected location to directly mount the fixator to the bone. Other external and less invasive permanent methods have used adhesives and/or adhesive-based components to externally mount a device to the skin of a patient. For example, conventional pin-less femur tracking arrays have used an adhesive draping placed over the edges of an array holder to affix the femur tracking arrays to patient soft tissue.

Temporary methods have included using releasably-coupled systems for attaching mounting devices to bone structures and have included the use of brackets configured to be tightened around the bone using mechanical notches, interlocking threads or teeth, a turn screw, a spindle screw, or a set screw. Releasably-coupled mounting systems are generally larger than fastener-affixed mounting devices and require more room to operate the elements required to tighten the brackets to the bone. In addition, tightening elements of conventional releasably-coupled mounting systems are challenging to manipulate in the limited space of an internal surgical environment. Furthermore, conventional releasably-coupled methods systems require tightening hardware on a mounting device that is difficult to tighten sufficiently to achieve a proper hold without introducing a risk of bone fracture due to over-tightening.

A primary challenge of existing techniques for procedures requiring internally-installed medical aid devices results from limited visibility and access to target anatomy, which has restricted the size and functionality of medical aid devices. Conventional permanent methods using fastener-affixed systems for attaching mounting devices to patient bony anatomy have used structures that lack flexibility and necessitate substantive additional pre- and post-operation procedures (for instance, additional and/or larger incisions) beyond those required for traditional surgical procedures. Conventional temporary methods using releasably-coupled systems have used mounting devices that require extensive space requirements around the installation area, lack variability, and are difficult to install with accurate tightening force. As a result, major disadvantages of conventional methods result from mounting devices that require manual loading and/or input from a healthcare professional and substantive extraneous workflow requirements to install and finalize the location and orientation of the mounting device and/or associated medical aid device.

Thus, it would be beneficial to provide mounting apparatuses that provide the rigid attachment to anatomy of fastener-affixed apparatuses, while allowing for flexibility and ease-of-use within a smaller invasive area than provided by conventional methods.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure provides mounting apparatuses configured to mount a medical aid device to a portion of a human body. The mounting apparatus may include a clamping assembly configured to temporarily and rigidly couple a medical aid device to a bone structure of the human body. In some embodiments, the mounting apparatuses may include a magnetically-actuated mounting apparatus using a magnetically-actuated clamping assembly, a spring-actuated mounting apparatus using a spring-actuated clamping assembly, or a mechanically-actuated mounting assembly using a mechanically-actuated clamping assembly.

In some embodiments, a magnetically-actuated mounting apparatus may include a pair of opposing arms coupled to a magnetically-actuated clamping assembly. The magnetically-actuated clamping assembly may be placed in an active state responsive to generation of a magnetic clamping force. The magnetically-actuated clamping assembly may be placed in an inactive state responsive to removal of the magnetic clamping force. In the active state, the opposing arms may be compressed around the portion of the human body to rigidly affix the magnetically-actuated mounting apparatus to the portion of the human body. In the inactive state, the opposing arms may have freedom of movement to move in a releasing direction away from the portion of the human body.

In some embodiments, the magnetically-actuated clamping assembly may be associated with an actuator magnet having an actuator magnetic field. In various embodiments, the magnetic clamping force may be generated responsive to alignment of the actuator magnetic field. In exemplary embodiments, the magnetic clamping force may be removed responsive to misalignment of the actuator magnetic field. In some embodiments, the magnetically-actuated clamping assembly may be associated with a fixed magnet having a fixed magnetic field. In one embodiment, alignment of the actuator magnetic field may include alignment of the actuator magnetic field with the fixed magnetic field to produce a combined magnetic field. In some embodiments, alignment of the actuator magnetic field and the fixed magnetic may include opposing poles of the actuator magnetic field and the fixed magnetic facing each other.

In one embodiment, the magnetic clamping force may be generated responsive to alignment of the actuator magnetic field and the fixed magnetic field, and the actuator magnetic field and the fixed magnetic being located at a distance within a threshold distance.

In some embodiments, movement of the magnetically-actuated mounting apparatus rotationally about or axially along the portion of the human body may be prevented when the magnetically-actuated clamping assembly is in the active state. In various embodiments, movement of the magnetically-actuated mounting apparatus rotationally about or axially along the portion of the human body may be allowed when the magnetically-actuated clamping assembly is in the inactive state. In some embodiments, movement of the opposing arms away from the portion of the human body is prevented when the magnetically-actuated clamping assembly is in the active state.

The present disclosure provides a magnetically-actuated mounting apparatus configured to mount a medical aid device to a portion of a human body. The magnetically-actuated mounting apparatus may include a first arm and a second arm configured to be arranged around the portion of the human body. The magnetically-actuated mounting apparatus may include a magnetically-actuated clamping assembly having the first arm and the second arm coupled to opposite ends thereof. The magnetically-actuated clamping assembly may include a fixed magnet having a fixed magnetic field and an actuator associated with an actuator magnet having an actuator magnetic field. The actuator may be configured to move to an engaged position to align the fixed magnetic field and the actuator magnetic field to generate a magnetic clamping force, the magnetic clamping force to force the first arm toward the second arm in a clamping direction to rigidly affix the magnetically-actuated mounting apparatus to the portion of the human body. The actuator may also be configured to move to a disengaged position in which the fixed magnetic field and the actuator magnetic field are misaligned to remove the magnetic clamping force.

In various embodiments, the magnetically-actuated clamping assembly may include a first support element configured to rigidly merge with a second support element responsive to the magnetic clamping force. In exemplary embodiments, the first support element may have a female portion configured to receive a corresponding male portion of the second support element.

In some embodiments, the actuator may be arranged within the second support element. In various embodiments, the actuator may be configured to rotate about a transverse axis of the magnetically-actuated clamping assembly to move to the engaged position or the disengaged position. In some embodiments, the actuator magnet may be arranged within a magnet cavity of the actuator. In some embodiments, the actuator magnet may be arranged within the magnet cavity such that the actuator magnetic field rotates in a corresponding direction with rotation of the actuator.

In exemplary embodiments, the magnetically-actuated clamping assembly may include an adjuster slidably arranged within a first support element. In some embodiments, the adjuster may be configured to move longitudinally within the first support element in one of a clamping direction or a releasing direction. In various embodiments, the fixed magnet may be arranged within the adjuster such that the fixed magnet moves with the adjuster.

In some embodiments, movement of the adjuster may adjust a distance between the fixed magnetic field and the actuator magnetic field. In various embodiments, the adjuster may move to an engaged position in which the fixed magnetic field is within a threshold distance of the actuator magnetic field. In exemplary embodiments, the adjuster may move to a disengaged position in which the fixed magnetic field is outside of the threshold distance.

In some embodiments, the magnetic clamping force may be generated within the magnetically-actuated clamping assembly responsive to the actuator being in the engaged position and the adjuster being within the engaged position. In various embodiments, the magnetic clamping force may be generated within the magnetically-actuated clamping assembly responsive to the fixed magnetic field and the actuator magnetic field being aligned and the fixed magnetic field and the actuator magnetic field being within the threshold distance.

In some embodiments, movement of the magnetically-actuated mounting apparatus rotationally about or axially along the portion of the human body is prevented when the magnetically-actuated clamping assembly is in the active state. In various embodiments, movement of the magnetically-actuated mounting apparatus rotationally about or axially along the portion of the human body is allowed when the magnetically-actuated clamping assembly is in the inactive state. In some embodiments, movement of the first arm or the second arm away from the portion of the human body in a releasing direction may be prevented when the magnetically-actuated clamping assembly is in the active state. In exemplary embodiments, movement of first support element or the second support element in a releasing direction may be prevented when the magnetically-actuated clamping assembly is in the active state.

In some embodiments, the actuator may include a drive configured to receive a tool for manually moving the actuator between the engaged position and the disengaged position. In various embodiments, the adjuster may include a post protruding through a cavity of the first support element, the post may be configured to facilitate manual movement of the adjuster between the engaged position and the disengaged position.

In some embodiments, at least one of the first arm and the second arm may include or may be an offset arm. In various embodiments, the offset arm may include a foot and a swivel connected via a ball-and-socket joint. In some embodiments, at least one of the first arm and the second arm may include at least one protrusion to facilitate attachment of at least one of the first arm or the second arm to the portion of the human body. In various embodiments, the at least one protrusion may include at least one claw structure.

In various embodiments, the medical aid device may be arranged in one of a plurality of cavities of one of the first support element or the second support element to facilitate variable placement of the medical aid device. In some embodiments, the medical aid device may be associated with an auxiliary magnet to affix a medical aid device within one of the first support element or the second support element.

The present disclosure provides a femur clamp to mount a tracking array to a femur. The femur clamp may include a first arm, a second arm, and a magnetically-actuated clamping assembly. The magnetically-actuated clamping assembly may have the pair of opposing arms coupled to opposite ends thereof. The magnetically-actuated clamping assembly may include a first indexer having a first proximal end configured to engage a second proximal end of a second indexer responsive to a magnetic clamping force within the magnetically-actuated clamping assembly. An actuator may be arranged within the magnetically-actuated clamping assembly to move to at least one engaged position to provide the magnetic clamping force within the magnetically-actuated clamping assembly to force the first arm and the second arm together in a clamping direction around the femur, and move to at least one disengaged position to remove the magnetic clamping force to allow at least one of the first arm or the second arm to move apart in a releasing direction.

The present disclosure provides methods for activating and deactivating a magnetically-actuated mounting apparatus configured to mount a medical aid device to a portion of a human body. In one embodiment, a method for activating the magnetically-actuated mounting apparatus may include placing the arms of a magnetically-actuated mounting apparatus around the portion of the human body, moving an actuator to an engaged position to align a magnetic field of an actuator magnet with a magnetic field of a fixed magnet, and moving the fixed magnet within a threshold distance of the actuator magnet to produce a combined magnetic field to generate a magnetic clamping force to force the arms around the portion of the human body.

In some embodiments, a method of deactivating the magnetically-actuated mounting apparatus may include one of moving actuator to a disengaged position to misalign the magnetic field of the actuator magnet with the magnetic field of the fixed magnet or moving the fixed magnet outside of a threshold distance of the actuator magnet to break up the combined magnetic field and remove the magnetic clamping force to allow the arms to move in a releasing direction.

The present disclosure provides a spring-actuated mounting apparatus configured to mount a medical aid device to a portion of a human body. The spring-actuated mounting apparatus may include a pair of opposing arms coupled to a spring-actuated clamping assembly. The spring-actuated clamping assembly may include a pin extending through a connection end of each of the pair of opposing arms and a spring arranged around a portion of the pin, each of the pair of opposing arms may be configured to rotate in one of a clamping direction or a releasing direction about the pin. The spring may include a pair of hooks extending away from a central body of the spring, each of the pair of hooks may be arranged to engage a portion of one of the pair of arms to bias an engagement end of each of the pair of arms in the clamping direction toward the portion of the human body.

In some embodiments, the spring-actuated mounting apparatus may include a holding device to hold a medical aid device. In some embodiments, the medical aid device may be or may include a tracking array for a computer-assisted surgical procedure. In various embodiments, the medical aid device may include one or more of a tracking array, a sensor, an image capturing device, a video capturing device, a logic device, or a wireless transmitter/receiver device. In some embodiments, the sensor may include a temperature sensor, a pressure sensor, an accelerometer sensor, or a gyroscopic sensor.

In one embodiment, the portion of the human body may include a bone structure. In various embodiments, the bone structure may include a femur. In some embodiments, the bone structure may include a shaft of a femur. In exemplary embodiments, the bone structure may include a medial portion of a femur. In some embodiments, the bone structure may include a lesser trochanter region. In various embodiments, the bone structure may include an anterior face of a proximal femur superior to a lesser trochanter.

In some embodiments, each of the pair of arms may include a set of prongs, and each of the set of prongs may include an opening to receive the pin. In various embodiments, the pin may include a barrel configured to receive a corresponding fastener. In some embodiments, the barrel may be internally threaded and the fastener may include a screw configured to be threaded into the barrel.

In various embodiments, the spring-actuated clamping assembly may include a pin having the central body of a torsion spring arranged around a longitudinal axis of the pin and a pair of hooks extending from the central body to engage the pair of opposing arms. In various embodiments, the pin may include a pair of flanges to hold arms in place longitudinally along pin. In various embodiments, the pair of flanges may include a barrel flange of a barrel of pin. In some embodiments, the pair of flanges may include a head of a screw threaded into the barrel of pin.

In some embodiments, the pair of opposing arms may include an anterior arm and a posterior arm. In various embodiments, the anterior arm may be configured to engage an anterior side of a lesser trochanter region of a femur. In exemplary embodiments, the posterior arm may be configured to engage a posterior side of the lesser trochanter region of a femur. In some embodiments, the posterior arm may be bifurcated to form a pair of claws. In various embodiments, the pair of claws may be positioned to straddle a portion of the posterior side of the lesser trochanter region. In some embodiments, the pair of claws may be positioned to straddle a portion of a bone structure anterior face of a proximal femur superior to a lesser trochanter.

In some embodiments, at least one of the pair of opposing arms may include a release attachment configured to receive a release device to place the spring-actuated mounting apparatus in an open position. In various embodiments, the release device may include a retractor. In some embodiments, the retractor may include a cobra retractor, a Gelpi retractor, or a device the same or similar to a cobra retractor or a Gelpi retractor that may operate as a release device according to various embodiments.

The present disclosure provides a method of mounting a medical aid device to a portion of a human body. The method may include providing a spring-actuated mounting apparatus that may include a pair of opposing arms coupled to a spring-actuated clamping assembly. The spring-actuated clamping assembly may include a pin extending through a connection end of each of the pair of opposing arms and a spring arranged around a portion of the pin, each of the pair of opposing arms may be configured to rotate in one of a clamping direction or a releasing direction about the pin. The spring may include a pair of hooks extending away from a central body of the spring, each of the pair of hooks may be arranged to engage a portion of one of the pair of arms to bias an engagement end of each of the pair of arms in the clamping direction toward the portion of the human body. The method may include positioning the spring-actuated mounting apparatus in a closed position around the portion of the human body.

The present disclosure provides a method of manufacturing a spring-actuated mounting apparatus configured to mount a medical aid device to a portion of a human body. The method may include providing a spring-actuated mounting apparatus that may include a pair of opposing arms coupled to a spring-actuated clamping assembly. The method may include forming the spring-actuated clamping assembly to include a pin extending through a connection end of each of the pair of opposing arms and a spring arranged around a portion of the pin, each of the pair of opposing arms may be configured to rotate in one of a clamping direction or a releasing direction about the pin. The method may including forming the spring to include a pair of hooks extending away from a central body of the spring, each of the pair of hooks may be arranged to engage a portion of one of the pair of arms to bias an engagement end of each of the pair of arms in the clamping direction toward the portion of the human body.

The present disclosure provides a mechanically-actuated mounting apparatus configured to mount a medical aid device to a portion of a human body. The mechanically-actuated mounting apparatus may include one of a linkage-tensioning mounting apparatus, a rack-and-pinion mounting apparatus, or a lever-locking mounting apparatus.

The linkage-tensioning mounting apparatus may include a pair of opposing arms coupled to a linkage-tensioning clamping assembly. The linkage-tensioning clamping assembly may include a tensioning mechanism configured to engage a linkage coupled to a connection end of at least one of the pair of opposing arms. The tensioning mechanism may operate to tighten the linkage to force the at least one of the pair of opposing arms in a clamping direction toward the portion of the human body. The tensioning mechanism may operate to release the linkage to allow the at least one of the pair of opposing arms to move in a releasing direction away from the portion of the human body.

In some embodiments of the linkage-tensioning mounting apparatus, the linkage may include a cable. In exemplary embodiments of the linkage-tensioning mounting apparatus, the tensioning mechanism may include a ratcheting system to tension the linkage via a ratcheting mechanism. In various embodiments, the ratcheting system may include a ratchet device having a drive shaft coupled to a spool holding at least a portion of the linkage. In exemplary embodiments, the ratchet device may be rotated in a tensioning direction to tension linkage to move one of the opposing arms in the clamping direction. In various embodiments, the ratchet device may be rotated in a relaxing direction to release the tension to allow one of the opposing arms to move in the releasing direction.

In some embodiments of the linkage-tensioning mounting apparatus, the ratchet device may be arranged within a housing. In various embodiments of the linkage-tensioning mounting apparatus, the ratchet device may include at least one tooth and the housing may include a set of internal teeth configured to engage the at least one tooth to prevent rotation of the ratchet device in the relaxing direction. In some embodiments, the ratchet device may be operably coupled to or include a release mechanism. In various embodiments, the release mechanism may allow the ratchet device to rotate in the relaxing direction.

In some embodiments of the linkage-tensioning mounting apparatus, the pair of opposing arms may be configured as free-arms. In various embodiments of the linkage-tensioning mounting apparatus, the free-arms may only be coupled via the linkage. In some embodiments of the linkage-tensioning mounting apparatus, the pair of opposing arms may include track-arms coupled via a post on a first arm configured to be arranged within a cylinder of a second arm.

The present disclosure provides a rack-and-pinion mounting apparatus configured to mount a medical aid device to a portion of a human body. The rack-and-pinion mounting apparatus may include a pair of opposing arms coupled to a rack-and-pinion clamping assembly. The rack-and-pinion clamping assembly may include a tensioning mechanism configured to engage a portion of a connection end of at least one of the pair of opposing arms. The tensioning mechanism may include a pinion operative to engage at least one rack of the at least one of the pair of opposing arms. Rotation of the pinion in a tensioning direction may cause the at least one of the pair of opposing arms to move in a clamping direction toward the portion of the human body. Rotation of the pinion in a relaxing direction may cause the at least one of the pair of opposing arms to move in a releasing direction away from the portion of the human body.

In some embodiments of the rack-and-pinion mounting apparatus, the rack-and-pinion clamping assembly may include a ratchet device configured to rotate in a tensioning direction to cause rotation of the pinion in the tensioning direction. In its nominal position, the ratchet device only rotates in the tensioning direction and prevents the pinion from rotating in a releasing direction. In some embodiments of the rack-and-pinion mounting apparatus, the rack-and-pinion clamping assembly may include a release mechanism to allow the ratchet device to rotate in a relaxing direction and allow the pinion to rotate in the relaxing direction.

In some embodiments of the rack-and-pinion mounting apparatus, the rack-and-pinion clamping assembly may include a ratchet pawl configured to prevent rotation of the pinion gear in the relaxing direction. In various embodiments of the rack-and-pinion mounting apparatus, the rack-and-pinion clamping assembly may include a biasing element configured to bias ratchet pawl in a direction to prevent rotation of the pinion gear in the relaxing direction. In some embodiments of the rack-and-pinion mounting apparatus, the rack-and-pinion clamping assembly may include a release element configured to move into an open position to disengage ratchet pawl from preventing rotation of the pinion gear in the relaxing direction to allow pinion to move in the relaxing direction. In exemplary embodiments of the rack-and-pinion mounting apparatus, the rack portion may be or may include a cylindrical rack. In some embodiments, the rack-and-pinion mounting apparatus may include a housing having the rack-and-pinion clamping assembly arranged therein, and the pair of opposing arms may include a first arm integral to the housing and a second arm having the rack portion.

In some embodiments of the rack-and-pinion mounting apparatus, the rack-and-pinion clamping assembly may include an anti-rotation hub comprising anti-rotation teeth, and the pinion gear may include ratchet kick teeth operative to engage the anti-rotation teeth to prevent rotation of the pinion gear in the relaxing direction when the anti-rotation hub is in a closed position. In some embodiments of the rack-and-pinion mounting apparatus, the anti-rotation teeth may be configured to allow rotation of the pinion gear in the tensioning direction when the anti-rotation hub is in the closed position. In some embodiments of the rack-and-pinion mounting apparatus, the anti-rotation hub may be configured to be moved into an open position to disengage anti-rotation teeth from the ratchet kick teeth to allow the pinion gear to rotate in the relaxing direction.

The present disclosure provides a lever-locking mounting apparatus configured to mount a medical aid device to a portion of a human body. The lever-locking mounting apparatus may include a pair of opposing arms coupled to a lever-locking clamping assembly. The lever-locking clamping assembly may include a locking mechanism and a tensioning mechanism coupled via a connector extending through a connection end of each of the pair of opposing arms. The tensioning mechanism may be configured to move in a tensioning direction to force at least one of the pair of opposing arms to move in the clamping direction toward the portion of the human body. The tensioning mechanism may be configured to move in a relaxing direction to allow the at least one of the pair of opposing arms to move in the releasing direction away from the portion of the human body. The locking mechanism may be configured to move into a locked position to fixate the mechanically-actuated mounting apparatus to the portion of the human body.

In various embodiments of the lever-locking mounting apparatus, the connector may include a threaded shaft. In some embodiments of the lever-locking mounting apparatus, the tensioner may include a wing nut. In some embodiments of the lever-locking mounting apparatus, the locking mechanism may include a cam lever. In various embodiments, the cam lever may be arranged in one of a horizontal position or a vertical position. In some embodiments of the lever-locking mounting apparatus, each of the opposing arms may include a prong having an opening configured to receive the connector.

In some embodiments of the lever-locking mounting apparatus, the tensioning mechanism may include a bevel-gear tensioner. In various embodiments of the lever-locking mounting apparatus, the bevel-gear tensioner may allow movement of the bevel-gear tensioner in the tensioning direction and the relaxing direction from above the mechanically-actuated clamping assembly. In some embodiments of the lever-locking mounting apparatus the bevel-gear tensioner may include a threaded bevel gear threaded onto the connector in a first orientation and a bevel-gear tensioner arranged in a second orientation perpendicular to the first orientation, the bevel-gear tensioner configured to engage the threaded bevel gear such that rotation of the bevel-gear tensioner causes a corresponding rotation of the threaded bevel gear.

In some embodiments of the described mechanically-actuated mounting apparatuses, a medical aid device may be coupled to the mechanically-actuated mounting apparatus. In various embodiments of the described mechanically-actuated mounting apparatuses, the medical aid device may be or may include a tracking array for a computer-assisted surgical procedure. In various embodiments of the described mechanically-actuated mounting apparatuses, the medical aid device may include one or more of a tracking array, a sensor, an image capturing device, a video capturing device, a logic device, or a wireless transmitter/receiver device. In some embodiments of the described mechanically-actuated mounting apparatuses, the sensor may include a temperature sensor, a pressure sensor, an accelerometer sensor, or a gyroscopic sensor.

In various embodiments of the described mechanically-actuated mounting apparatuses, the medical aid device may be coupled to the mechanically-actuated mounting apparatus via at least one mounting point of a holding device. In some embodiments of the described mechanically-actuated mounting apparatuses, the holding device may include a plurality of mounting points to facilitate mounting of a plurality of medical aid devices or to allow for placement of the medical aid device in a plurality of positions about mounting device.

In one embodiment of the described mechanically-actuated mounting apparatuses, the portion of the human body may include a bone structure. In various embodiments of the described mechanically-actuated mounting apparatuses, the bone structure may include a femur. In some embodiments of the described mechanically-actuated mounting apparatuses, the bone structure may include a shaft of a femur. In exemplary embodiments of the described mechanically-actuated mounting apparatuses, the bone structure may include one of a proximal portion of a femur.

In some embodiments of the described mechanically-actuated mounting apparatuses, the pair of opposing arms may include a medial arm and a lateral arm. In various embodiments of the described mechanically-actuated mounting apparatuses, the medial arm may be configured to engage a medial side of a femur. In exemplary embodiments of the described mechanically-actuated mounting apparatuses, the lateral arm may be configured to engage a lateral side of the femur.

In some embodiments of the described mechanically-actuated mounting apparatuses, both of the pair of opposing arms are engaged with mechanically-actuated clamping assembly such that mechanically-actuated clamping assembly may move both of the pair of opposing arms in a clamping direction and/or a releasing direction. In some embodiments of the described mechanically-actuated mounting apparatuses, one of the pair of opposing arms may be a fixed arm that is not moved by the mechanically-actuated clamping assembly.

The present disclosure provides a method of mounting a medical aid device to a portion of a human body via a linkage-tensioning mounting apparatus. The method may include providing the linkage-tensioning mounting apparatus having a pair of opposing arms coupled to a linkage-tensioning clamping assembly. The linkage-tensioning clamping assembly may include a tensioning mechanism configured to engage a linkage coupled to a connection end of at least one of the pair of opposing arms. The method may include operating the tensioning mechanism to tighten the linkage to force the at least one of the pair of opposing arms in a clamping direction toward the portion of the human body. The method may include operating the tensioning mechanism to release the linkage to allow the at least one of the pair of opposing arms to move in a releasing direction away from the portion of the human body.

The present disclosure provides a method of mounting a medical aid device to a portion of a human body via a rack-and-pinion mounting apparatus. The method may include providing the rack-and-pinion mounting apparatus having a pair of opposing arms coupled to a rack-and-pinion clamping assembly. The rack-and-pinion clamping assembly may include a tensioning mechanism configured to engage a portion of a connection end of at least one of the pair of opposing arms. The tensioning mechanism may include a pinion operative to engage at least one rack of the at least one of the pair of opposing arms. The method may include rotating the pinion in a tensioning direction to cause the at least one of the pair of opposing arms to move in a clamping direction toward the portion of the human body. The method may include rotating the pinion in a relaxing direction to cause the at least one of the pair of opposing arms to move in a releasing direction away from the portion of the human body.

The present disclosure provides a method of mounting a medical aid device to a portion of a human body via a lever-locking mounting apparatus. The method may include providing the lever-locking mounting apparatus having a pair of opposing arms coupled to a mechanically-actuated clamping assembly. The lever-locking clamping assembly may include a locking mechanism and a tensioning mechanism coupled via a connector extending through a connection end of each of the pair of opposing arms. The method may include operating the tensioning mechanism to move in a tensioning direction to force at least one of the pair of opposing arms to move in the clamping direction toward the portion of the human body. The method may include operating the tensioning mechanism to move in a relaxing direction to allow the at least one of the pair of opposing arms to move in the releasing direction away from the portion of the human body. The method may include operating the locking mechanism to move into a locked position to fixate the mechanically-actuated mounting apparatus to the portion of the human body.

In various embodiments, the medical aid device may be coupled to a mounting apparatus via at least one mounting point of a holding device. In some embodiments, the holding device may include a plurality of mounting points to facilitate mounting of a plurality of medical aid devices or to allow for placement of the medical aid device in a plurality of positions about the mounting device.

In some embodiments, the medical aid device may be or may include a tracking array for a computer-assisted surgical procedure. In various embodiments, the medical aid device may include one or more of a tracking array, a sensor, an image capturing device, a video capturing device, a logic device, or a wireless transmitter/receiver device. In some embodiments, the sensor may include a temperature sensor, a pressure sensor, an accelerometer sensor, or a gyroscopic sensor.

In one embodiment, the portion of the human body may include a bone structure. In exemplary embodiments, the bone structure may include a portion of a hip. In various embodiments, the bone structure may include a femur. In some embodiments, the bone structure may include a shaft of a femur. In exemplary embodiments, the bone structure may include a medial portion of a femur. In some embodiments, the bone structure may include a lesser trochanter region. In various embodiments, the bone structure may include an anterior face of a proximal femur superior to a lesser trochanter. In some embodiments, the portion of the human body may include a portion of a femur or portions of a femur, such as a medial portion of a femur, a femur shaft, a lesser trochanter region, a greater trochanter region, and/or the like.

Embodiments of the present disclosure provide numerous advantages. In one non-limiting example technological advantage, a mounting apparatus according to some embodiments may provide a clamping body that allows for rigid attachment to a variety of bony anatomy through a magnetically-actuated mechanism, a spring-actuated mechanism, and/or a mechanically-actuated (including, for instance, a linkage-tensioning mechanism, a lever-locking mechanism, or a rack-and-pinion mechanism).

In one non-limiting example advantage, a mounting apparatus having a clamping assembly according to some embodiments may require less space and may be easier to install, position, and/or orient than conventional apparatuses. In an additional non-limiting example advantage, a mounting apparatus according to some embodiments may be more flexible and configurable to accommodate a wider range of patient anatomical variances than conventional apparatuses, while still being able to temporarily and rigidly attach to target anatomical structures. For instance, a magnetically-actuated mounting apparatus according to some embodiments may provide a clamping body that allows for rigid attachment to a variety of bony anatomy through a loss motion mechanism.

In a further non-limiting example advantage, clamping assemblies according to some embodiments may allow for simple, efficient clamping and releasing of a mounting apparatus without requiring the substantive pre- and post-procedure steps required of conventional systems and techniques.

In a still further non-limiting example advantage, a mounting apparatus according to some embodiments may include an indexer with variable installation points for a medical aid device and/or that may be articulated about a mounting apparatus to facilitate simple and efficient variable positioning of a medical aid device compared with conventional apparatuses. In another non-limiting example advantage, mounting apparatuses according to some embodiments may not require any screws and/or adhesives, in contrast with conventional apparatuses.

In an additional non-limiting example advantage, mounting apparatuses according to some embodiments may mitigate risk to disruption to blood flow and soft tissue surrounding a bone target site and fracturing of the bone target. In a further non-limiting example advantage, mounting apparatuses according to some embodiments may provide a clamping body that enables multiple degrees of freedom around a central axis for optimal application of the magnetically-actuated mounting apparatus and positioning of a medical aid device (for instance, a tracking array).

In another non-limiting example technological advantage, a mounting apparatus according to some embodiments may be rigidly attached to a portion of a human body through an external (for instance, magnetically-actuated, spring-actuated, and/or mechanically-actuated) clamping force that does not require permanent attachment techniques, such as screws or adhesive.

In one non-limiting example technological advantage, mounting apparatuses according to some embodiments may operate using a clamping force as a fixation method for mounting a medical aid device to an anatomical structure.

With particular respect to a hip replacement procedure, in a non-limiting example advantage, mounting apparatuses according to some embodiments may be compatible with a direct anterior approach. For example, clamping assemblies according to some embodiments may reduce or even eliminate additional femur preparation by allowing placement of the mounting apparatus in an incision created for the procedure (for instance, a proximal portion of the femur).

Further features and advantages of at least some of the embodiments of the present disclosure, as well as the structure and operation of various embodiments of the present disclosure, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 shows an example of a first operating environment that may be representative of some embodiments of the present disclosure;

FIG. 2 shows a block diagram of an example of an embodiment of a magnetically-actuated mounting apparatus in accordance with features of the present disclosure;

FIG. 3 shows examples of active and inactive configurations of an embodiment of a magnetically-actuated clamping assembly in accordance with features of the present disclosure;

FIG. 4A shows a perspective view of an example of an embodiment of a magnetically-actuated mounting apparatus in accordance with features of the present disclosure;

FIG. 4B shows a cross-sectional side view of the magnetically-actuated mounting apparatus shown in FIG. 4A;

FIG. 4C shows an exploded top view of the magnetically-actuated clamping assembly shown in FIG. 4A;

FIG. 4D shows an exploded side view of the magnetically-actuated clamping assembly shown in FIG. 4A;

FIG. 4E depicts a close-up view of an area of the magnetically-actuated mounting apparatus shown in FIG. 4A;

FIG. 5 shows active and inactive states of an embodiment of a magnetically-actuated clamping assembly in accordance with features of the present disclosure;

FIG. 6A shows a first perspective side view of an example of an embodiment of a magnetically-actuated mounting apparatus with an offset arm in accordance with features of the present disclosure;

FIG. 6B shows a second perspective side view of the magnetically-actuated mounting apparatus shown in FIG. 6A;

FIG. 6C shows a cross-sectional side view of the magnetically-actuated mounting apparatus shown in FIG. 6A;

FIG. 6D shows an exploded, perspective side view of the offset arm of the magnetically-actuated mounting apparatus shown in FIG. 6A;

FIG. 7 depicts a method in accordance with embodiments of the disclosure.

FIG. 8A shows a side view of a block diagram of an example of an embodiment of a spring-actuated mounting apparatus in accordance with features of the present disclosure;

FIG. 8B shows a top view a top view of a block diagram of an example of an embodiment of a spring-actuated mounting apparatus in accordance with features of the present disclosure;

FIG. 9A shows a first perspective view of an example of an embodiment of a spring-actuated mounting apparatus in accordance with features of the present disclosure;

FIG. 9B shows a second perspective view of the spring-actuated mounting apparatus shown in FIG. 9A;

FIG. 9C shows an exploded view of the spring-actuated clamping assembly shown in FIG. 9A;

FIG. 10 shows an example of an embodiment of a spring-actuated mounting apparatus attached to a portion of a femur in accordance with features of the present disclosure;

FIGS. 11A and 11B show perspective views of an example embodiment of an arm of a spring-actuated mounting apparatus in accordance with features of the present disclosure;

FIG. 12 shows a perspective view of an example embodiment of a release device and a spring-actuated mounting apparatus in accordance with features of the present disclosure;

FIG. 13 shows a side view of a block diagram of an example of an embodiment of a linkage-tensioning mounting apparatus in accordance with features of the present disclosure;

FIG. 14A shows a side view of a ratchet-based tensioning mechanism of a linkage-tensioning mounting apparatus in accordance with features of the present disclosure;

FIG. 14B shows a perspective view of a ratchet device of the tensioning mechanism of FIG. 14A;

FIG. 14C shows a perspective view of a housing of the tensioning mechanism of FIG. 14A;

FIG. 15A shows a side view of a first embodiment of a free-arm linkage-tensioning mounting apparatus in accordance with features of the present disclosure;

FIG. 15B shows an internal side view of arms of the linkage-tensioning mounting apparatus of FIG. 15A;

FIG. 16A shows a perspective view of a second embodiment of a free-arm linkage-tensioning mounting apparatus in accordance with features of the present disclosure;

FIG. 16B shows a perspective view of a tensioning mechanism for the free-arm linkage-tensioning mounting apparatus of FIG. 16A;

FIG. 17A shows a side view of a track-arm linkage-tensioning mounting apparatus in accordance with features of the present disclosure;

FIG. 17B shows a perspective view of a first embodiment of track-arms for a track-arm linkage-tensioning mounting apparatus in accordance with features of the present disclosure;

FIG. 17C shows a perspective view of a second embodiment of track-arms for a track-arm linkage-tensioning mounting apparatus in accordance with features of the present disclosure;

FIG. 18A shows a perspective view of a first embodiment of a rack-and-pinion mounting apparatus in accordance with features of the present disclosure;

FIG. 18B shows a perspective view of a tensioning mechanism for the rack-and-pinion mounting apparatus of FIG. 18A;

FIGS. 19A and 19B show top-down views of a second embodiment of a rack-and-pinion mounting apparatus in an open configuration in accordance with features of the present disclosure;

FIGS. 19C and 19D show top-down views of the second embodiment of a rack-and-pinion mounting apparatus in a closed configuration in accordance with features of the present disclosure;

FIG. 19E shows a side view of the rack-and-pinion mounting apparatus of FIG. 19A;

FIG. 19F shows a side view and a perspective view of a cylindrical rack arm of the rack-and-pinion mounting apparatus of FIG. 19A;

FIG. 20A shows a perspective view and a cross-sectional view of a third embodiment of a rack-and-pinion mounting in accordance with features of the present disclosure;

FIG. 20B shows a pinion gear of the rack-and-pinion mounting apparatus of FIG. 20A;

FIG. 21A shows a perspective view of a fourth embodiment of a rack-and-pinion mounting apparatus in accordance with features of the present disclosure;

FIG. 21B shows a side view of the rack-and-pinion mounting apparatus of FIG. 21A;

FIG. 21C shows a pinion gear of the rack-and-pinion mounting apparatus of FIG. 21A;

FIG. 22A shows a side view of a lever-locking mounting apparatus in accordance with features of the present disclosure;

FIG. 22B shows an exploded side view of the lever-locking mounting apparatus of FIG. 22A;

FIG. 22C shows a locking/unlocking process for the lever-locking mounting apparatus of FIG. 22A;

FIG. 22D shows a side view of the lever-locking mounting apparatus of FIG. 22A in a side-locking position;

FIG. 22E shows a top view of the lever-locking mounting apparatus of FIG. 22A in a side-locking position;

FIG. 23 shows a side view of a bevel-gear configuration of a lever-locking mounting apparatus in accordance with features of the present disclosure;

FIGS. 24A-24G show perspective views of example embodiments of arms of a mounting apparatus in accordance with features of the present disclosure; and\

FIG. 25 shows cross sections of bone anatomical structures.

The drawings are not necessarily to scale. The drawings are merely representations, not intended to portray specific parameters of the disclosure. The drawings are intended to depict example embodiments of the disclosure, and therefore are not to be considered as limiting in scope. In the drawings, like numbering represents like elements.

Furthermore, certain elements in some of the figures may be omitted, or illustrated not-to-scale, for illustrative clarity. The cross-sectional views may be in the form of “slices”, or “near-sighted” cross-sectional views, omitting certain background lines otherwise visible in a “true” cross-sectional view, for illustrative clarity. Furthermore, for clarity, some reference numbers may be omitted in certain drawings.

### DETAILED DESCRIPTION

Embodiments of an improved anatomical structure mounting apparatus will now be described more fully hereinafter with reference to the accompanying drawings, in which preferred embodiments of the present disclosure are presented. As will be described and illustrated, in some embodiments, the improved anatomical structure mounting apparatus may include one of a magnetically-actuated mounting apparatus, a spring-actuated mounting apparatus, or a mechanically-actuated mounting apparatus.

In some embodiments, a magnetically-actuated mounting apparatus may include a pair of arms, a magnetically-actuated clamping assembly having a magnet-based mechanism to temporarily and rigidly compress the pair of arms around the anatomical structure, and at least one support element to hold a medical aid device proximate to the anatomical structure.

Thus arranged, as will be described in greater detail, the magnetically-actuated mounting apparatus according to some embodiments provides the ability to temporarily yet rigidly mount a medical aid device to an internal anatomical structure of a patient in a compact area, while being flexible to accommodate a wide range of anatomical variances between patients in a simple, adjustable, and easily activated/deactivated configuration.

In one embodiment, for example, the described technology may use a combination of magnetic power and stiffness of mechanical members to provide a magnetically-actuated mounting apparatus (or clamp) that accepts a variety of bony anatomy. Magnetic poles within the clamp connection may provide a linear clamping force to mount the clamp around, for instance, a femoral shaft. In the inactive state, the clamp may provide adjustability for a variety of bony anatomy. In the active state, rigidity is provided through the magnet-based connection that may prevent all or substantially all rotational and axial degrees of freedom. As such, a tracking array can be placed on the clamp in various positions, for example, to allow for the best opportunity for detection by a detection device (for instance, a navigation camera).

In one embodiment, for example, the magnetically-actuated mounting apparatus may be in the form of a variable anatomic femur clamp having an indexing, magnetic clamp connection that allows a magnetic force to overcome a linear spring force causing two support or indexing bodies to merge. A break in the magnetic field may occur when a separate shaft or actuator is moved to misalign a portion of the magnetic field of the device, thereby significantly reducing clamping force and allowing disassembly of the device.

As will be described and illustrated, in some embodiments, an improved anatomical structure mounting apparatus may include a pair of arms (or claws), a spring-actuated clamping assembly having a spring-actuated mechanism to temporarily and rigidly compress the pair of arms around the anatomical structure, and at least one support element to hold a medical aid device proximate to the anatomical structure.

Thus arranged, as will be described in greater detail, the spring-actuated mounting apparatus according to some embodiments provides the ability to temporarily yet rigidly mount a medical aid device to an internal anatomical structure of a patient in a compact area, while being flexible enough to mitigate damage to the anatomical structure and accommodate a wide range of anatomical variances between patients in a simple, adjustable, and easily attachable/detachable configuration.

In one embodiment, for example, the described technology may use a combination of spring-actuated forces and stiffness of mechanical members to provide a spring-actuated mounting apparatus (or clamp) that may be installed on a variety of bony anatomy. The spring-actuated mounting apparatus may include a pair of arms configured to clamp around an anatomical structure. In some embodiments, each of the pair of arms may include a connecting end configured to have a pin or rod extend therethrough such that each of the arms may rotate or pivot about the pin. A spring mechanism may be arranged around the pin. In various embodiments, the spring mechanism may have a central body formed of coils and hooks extending from the central body to engage each of the arms to bias, force, or push the arms toward the anatomical structure to clamp the spring-actuated mounting apparatus to the anatomical structure. In some embodiments, for example, a spring-actuated mounting apparatus may be affixed to an anatomical structure via a spring-actuated clamping assembly that actuates a clamping force by using a single pin connection and a torsion spring.

In some embodiments, the portion of the human body may include a portion of a femur or portions of a femur, such as a medial portion of a femur, a femur shaft, a lesser trochanter region, a greater trochanter region, and/or the like. In various embodiments, the opposing arms may include an anterior arm configured to engage a first side of the lesser trochanter region and a posterior arm configured to engage a second side, opposite the first side, of the lesser trochanter region. In some embodiments, the first side may be an anterior side of the lesser trochanter and the second side may be a posterior side of the lesser trochanter. One or both of the opposing arms may include teeth to dig into the femur to further facilitate attachment of the spring-actuated mounting apparatus. A portion of the spring-actuated mounting apparatus may include a device holder configured to hold a medical aid device directly adjacent to the anatomical structure when the spring-actuated mounting apparatus is installed at the target mounting site. In some embodiments, the medical aid device may include a tracking array, for example, as a navigation guide for computer-assisted surgery.

As will be described and illustrated, in some embodiments, the improved anatomical structure mounting apparatus may include a pair of arms (or claws) and a mechanically-actuated clamping assembly having a linkage-tensioning mechanism, a rack-and-pinion mechanism, or a lever-locking mechanism to temporarily and rigidly compress the pair of arms around the anatomical structure.

Thus arranged, as will be described in greater detail, the mechanically-actuated mounting apparatus according to some embodiments provides the ability to temporarily yet rigidly mount a medical aid device to an internal anatomical structure of a patient in a compact area, while being flexible enough to mitigate damage to the anatomical structure and accommodate a wide range of anatomical variances between patients in a simple, adjustable, and easily attachable/detachable configuration.

In one embodiment, for example, the described technology may use a combination of linkage-tensioning forces and stiffness of mechanical members to provide a mechanically-actuated mounting apparatus (or clamp) that may be installed on a variety of bony anatomy. The linkage-tensioning mounting apparatus may include a pair of opposing arms configured to clamp around an anatomical structure. In some embodiments, at least one of the pair of opposing arms may include a connecting end coupled to a linkage. Non-limiting examples of a linkage may include a cable or a wire. The linkage may be coupled to a tensioning mechanism, such as a ratchet or ratchet system. The tensioning mechanism may be coupled or otherwise engaged with the linkage. The tensioning mechanism may move to tighten the linkage, thereby causing at least one of the pair of opposing arms to compress or move in a clamping direction toward the portion of the human anatomy. In this manner, the mechanically-actuated clamping assembly may operate to bias, force, push, or hold the opposing arms toward the anatomical structure to clamp the mechanically-actuated mounting apparatus to the anatomical structure.

In one embodiment, for example, the described technology may use a combination of rack-and-pinion forces and stiffness of mechanical members to provide a mechanically-actuated mounting apparatus (or clamp) that may be installed on a variety of bony anatomy. The rack-and-pinion mounting apparatus may include a pair of opposing arms coupled to a rack-and-pinion clamping assembly. The rack-and-pinion clamping assembly may include a tensioning mechanism configured to engage a portion of a connection end of at least one of the pair of opposing arms. The tensioning mechanism may include a pinion operative to engage at least one rack of the at least one of the pair of opposing arms. Rotation of the pinion in a tensioning direction may cause the at least one of the pair of opposing arms to move in a clamping direction toward the portion of the human body. Rotation of the pinion in a relaxing direction may cause the at least one of the pair of opposing arms to move in a releasing direction away from the portion of the human body.

In another embodiment, for example, the described technology may use a combination of lever-locking forces and stiffness of mechanical members to provide a mechanically-actuated mounting apparatus (or clamp) that may be installed on a variety of bony anatomy. The mechanically-actuated mounting apparatus may include a pair of opposing arms configured to clamp around an anatomical structure. In some embodiments, the opposing arms may be coupled to a lever-locking clamping assembly. The lever-locking clamping assembly may include a locking mechanism and a tensioning mechanism coupled via a connector extending through a connection end of each of the pair of opposing arms. The tensioning mechanism may be configured to move in a tensioning direction to force at least one of the pair of opposing arms to move in the clamping direction toward the portion of the human body. The tensioning mechanism may be configured to move in a relaxing direction to release tension in the linkage and allow the at least one of the pair of opposing arms to move in the releasing direction away from the portion of the human body. The locking mechanism may be configured to move into a locked position to fixate the mechanically-actuated clamping assembly. The locking assembly may be moved into an unlocked position to allow movement of arms away from each other in a releasing direction.

In some embodiments, the portion of the human body may include a portion of a femur or portions of a femur, such as a femur shaft, a lesser and/or greater trochanter region, a proximal femur, a medial femur, a portion distal to a femoral neck cut, and/or the like. Although a femur may be used as an example of an anatomical structure herein, embodiments are not so limited, as any type of anatomical structure capable of being used as a mounting structure for apparatuses and/or methods according to some embodiments is contemplated herein. In some embodiments in which the portion of the human body is a femur (including the proximal end of the femur), a first of the opposing arms may be a medial arm configured to engage a medial side of the femur, and a second of the opposing arms may be a lateral arm configured to engage a lateral side of the femur. In some embodiments, at least one of the opposing arms may include an angled offset to allow for increased force on the portion of the human anatomy.

In exemplary embodiments, one or both of the opposing arms may include protrusions, such as teeth or a roughened surface, to dig into the femur to further facilitate attachment of the mechanically-actuated mounting apparatus. A portion of the mechanically-actuated mounting apparatus may include a device holder configured to hold a medical aid device directly adjacent to the anatomical structure when the mechanically-actuated mounting apparatus is installed at the target mounting site. In various embodiments, a medical aid device may be attached directly to a portion of the mechanically-actuated mounting apparatus, such as to an arm or portion of a mechanically-actuated clamping assembly. In some embodiments, the medical aid device may include a tracking array, for example, as a navigation guide for computer-assisted surgery.

In some linkage-tensioning embodiments, a mechanically-actuated clamping assembly or mechanism may use a ratcheting cable tensioner to squeeze a portion of the human anatomy, for example, the proximal femur. As the tensioner is turned, a linkage (for example, a cable) that is attached to at least one of two opposing arms or claws is tightened (for example, the length of the linkage outside of the tensioner decreases or shortens and/or via elastic forces of the linkage), thereby causing or increasing tension in the linkage. The tension in the linkage may draw the two arms together to fixate the mechanically-actuated mounting apparatus to the portion of the human anatomy. The tensioner may include a release mechanism that allows for the tensioner to disengage the tension and quickly allow the arms to separate, thereby releasing (or partially releasing) the mechanically-actuated mounting apparatus from the portion of the human anatomy.

In some lever-locking embodiments, a pair of opposing arms may be configured to grip both sides of a portion of the human anatomy, for instance, the proximal end of a femur. A mechanically-actuated clamping assembly may be used to generate a force that may be transferred to the opposing arms via turning a fastener about a connector extending between the opposing arms that may be hand or tool tightened. For example, the fastener may be a threaded nut configured to engage corresponding threads on a bolt connector. Turning the nut in a first direction may move the nut along the connector in a clamping direction toward the portion of the human anatomy, and turning the nut in a second direction may move the nut along the connector in a releasing direction away from the portion of the human anatomy. Movement of the nut may cause corresponding movement of at least one of the opposing arms. The mechanically-actuated clamping assembly and, therefore, the opposing arms, may be fixated through the levering action of a cam or other locking mechanism that prevents rotation or other movement of the fastener about the connector.

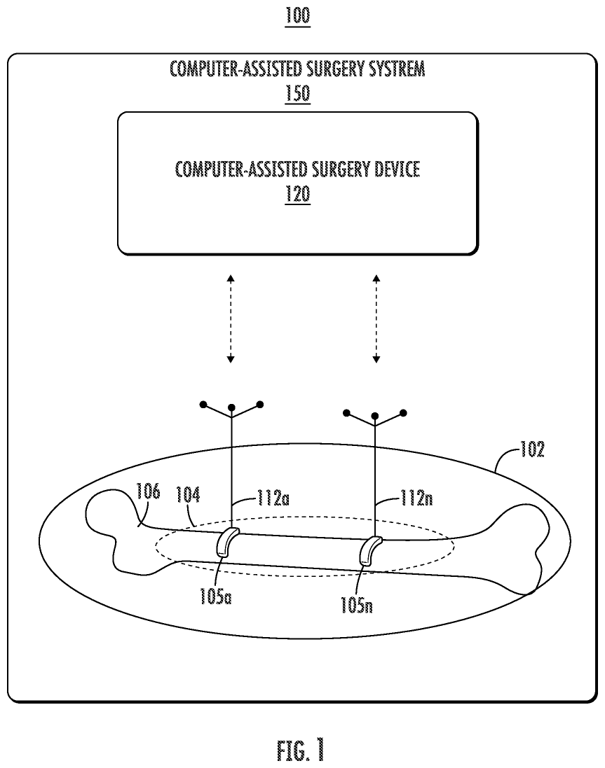

FIG. 1 illustrates an example of an operating environment that may be representative of some embodiments. As shown in FIG. 1, operating environment 100 may include a computer-assisted surgery (CAS) system 150 for performing computer- and/or robotic-assisted surgery on a patient, such as a computer-assisted navigated hip or knee replacement procedure. Although hip and knee replacement procedures are used as examples, embodiments are not so limited; any type of procedure capable of being performed using apparatuses and/or methods according to some embodiments is contemplated herein.

CAS 150 may include one or more types of computer-assisted surgical systems, devices, and/or the like, including, without limitation, image-guided systems, robotics systems, computer-assisted systems, navigation systems, and/or the like. Non-limiting examples of CAS 150 may be or may include Brainlab® surgery systems and NAVIO™ surgical robotics systems provided by Smith & Nephew of London, United Kingdom.

In some embodiments, CAS 150 may include a CAS device 120 configured to implement a computer-assisted surgical procedure on a patient. For example, CAS device 120 may by or may include a processor, logic device, computing device, and/or the like configured to at least partially perform a navigated hip or knee replacement procedure. Disclosure of a computing and/or logic device herein may generally be or include a device having a processor, controller, circuitry, and/or the like operative to execute instructions, including, without limitation, computer-readable instructions, program code, and/or the like stored or otherwise accessible by the device to perform the described function(s). CAS device 120 may be configured to communicate with various medical aid devices 112a-n mounted on a portion of a patient via mounting apparatuses 105a-n configured according to some embodiments.

In the example depicted in FIG. 1, medical aid devices 112a-n may include tracking arrays mounted on a femur 106 of a leg 102 of a patient. Although tracking arrays are used as examples of medical aid devices 112a-n, embodiments are not so limited; various types of medical aid devices may be used in accordance with embodiments described herein. In addition, embodiments are not limited to coupling mounting apparatuses 105 to femurs and/or shafts of femurs, as any portion of the human body capable of being coupled to mounting apparatuses 105 according to some embodiments is contemplated herein. Non-limiting examples of medical aid devices 112a-n may include sensors (including, without limitation, temperature sensors, pressure sensors, accelerometer sensors, gyroscopic sensors, and/or the like), image and/or video devices, logic devices, processors, controllers, circuitry, wireless transmitters/receivers, and/or the like. Tracking arrays 112a-n may operate as landmarks, guides, navigation elements, and/or the like to assist CAS device 120 in performing a navigated surgical procedure.

Medical aid devices 112a-n and/or mounting apparatuses 105a-n may be accessed via an incision 104 in leg 102. As described in more detail below, mounting apparatuses may be clamped to and/or released from femur 106 and the position and/or orientation of medical aid devices 112a-n and/or mounting apparatuses 105a-n may be adjusted in a simple, efficient manner that requires less space, time, and surgical steps than that required of conventional mounting apparatuses.

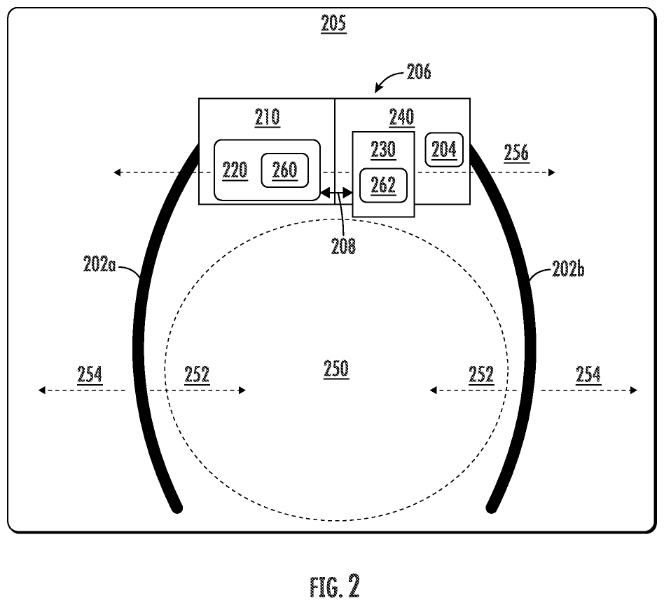

FIG. 2 shows a block diagram of an example embodiment of a magnetically-actuated mounting apparatus in accordance with features of the present disclosure. As shown in FIG. 2, magnetically-actuated mounting apparatus 205 may include a plurality of arms 202a and 202b, a medical aid device 204, and a magnetically-actuated clamping assembly 206. In some embodiments, magnetically-actuated clamping assembly 206 may operate to temporarily clamp, attach, affix, mount, or otherwise couple arms 202a and 202b to a portion of a human body 250, for example, a shaft of a femur.

In some embodiments, magnetically-actuated clamping assembly 206 may include a support element 210 (for example, a first support element) configured to interface with a support element 240 (for example, a second support element). Arm 202a may be attached to an end of support element 210, and arm 202b may be attached to an opposing end of support element 240. In various embodiments, arms 202a and 202b may be configured as opposing arms operative to engage opposite sides of femur 250.

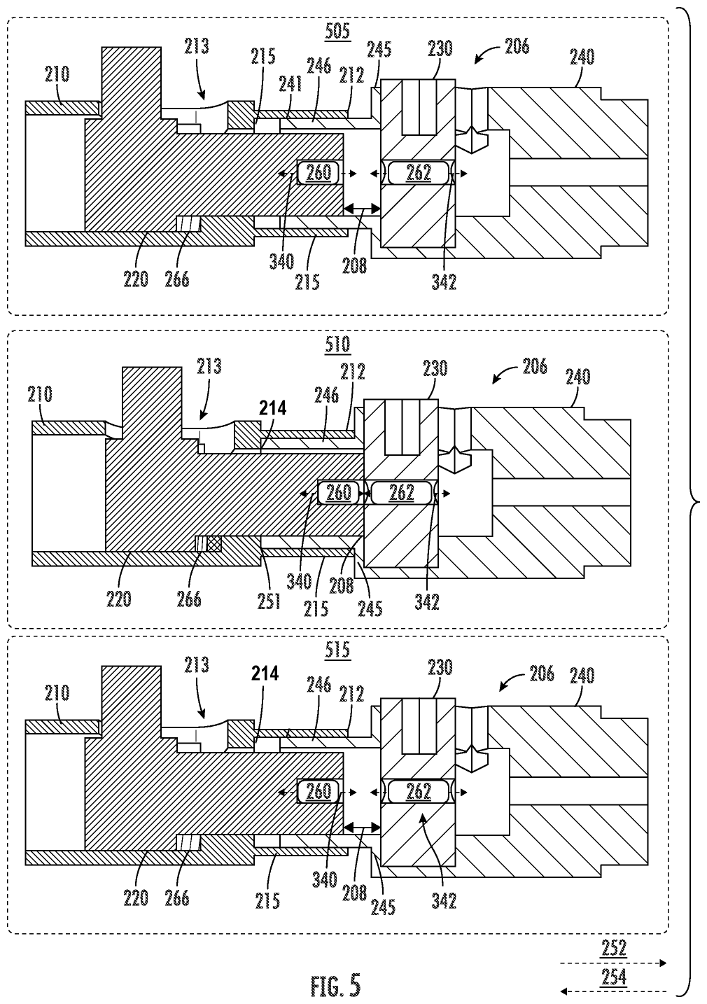

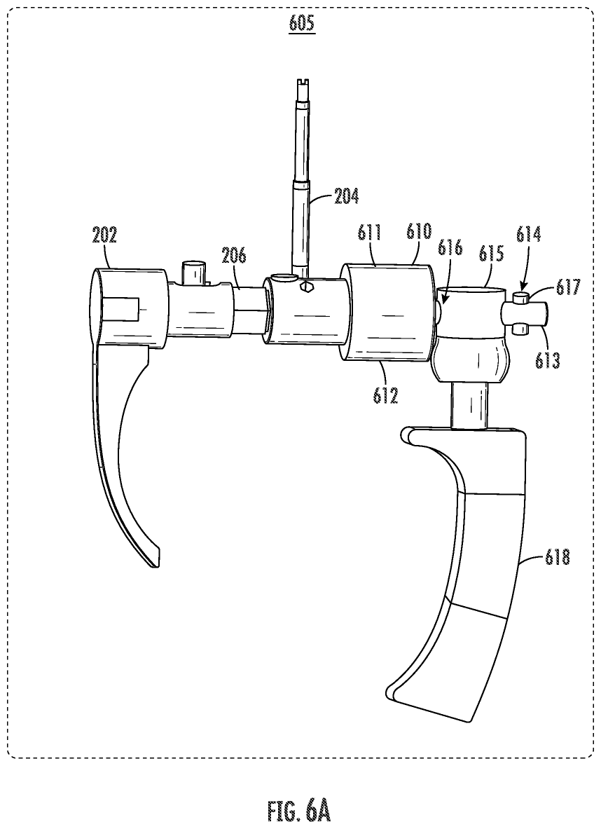

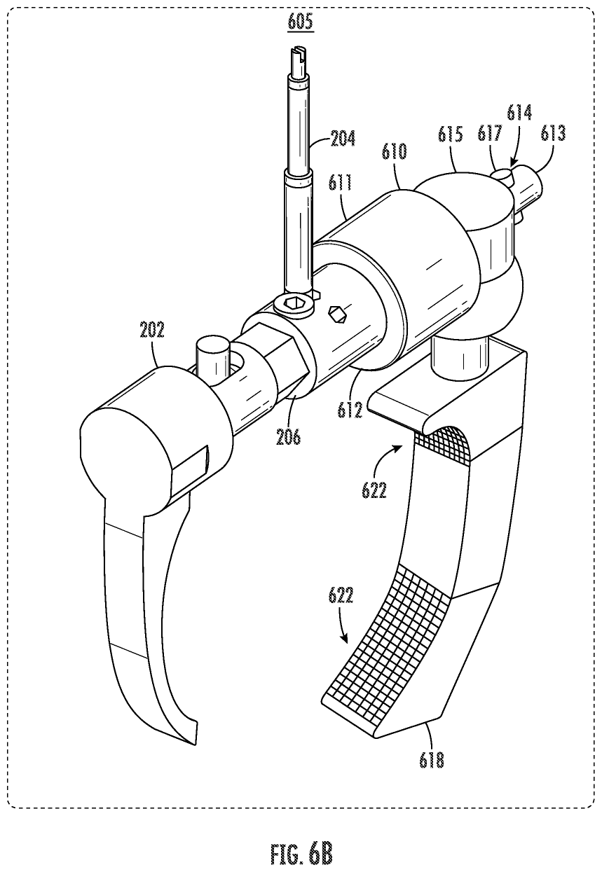

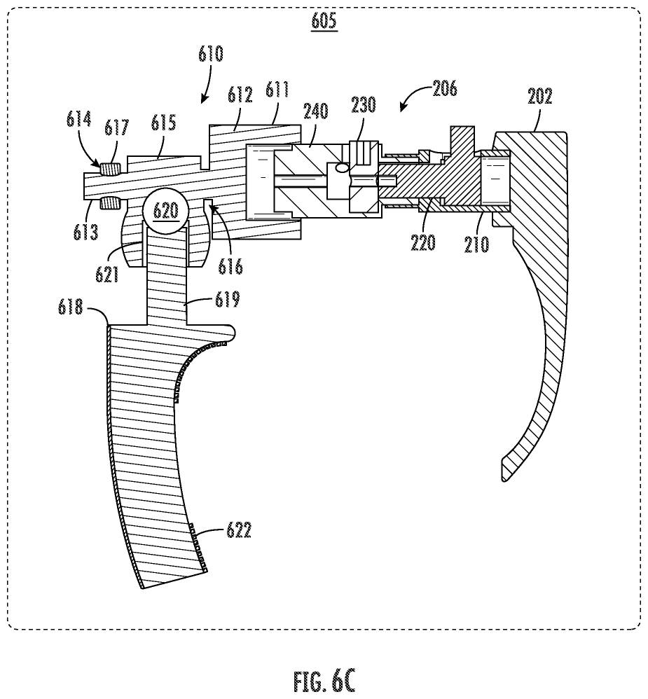

In various embodiments, at least one of arms 202a and 202b may be configured to be articulated about magnetically-actuated clamping assembly 206. For example, arm 202a and/or arm 202b may be rotatable about mounting assembly 206, for example, about longitudinal axis 256. In some embodiments, at least one of arms 202a and 202b may be configured to move, flex, pivot, articulate, or otherwise be manipulated with respect to magnetically-actuated clamping assembly 206. For example, arm 202a and/or arm 202b may have various degrees of freedom of movement about magnetically-actuated clamping assembly 206 and/or support elements 210 and 240, respectively. For instance, arms 202a and/or arm 202b may be configured to pivot up and/or down, left and/or right, and/or directions therebetween about support elements 210 and 240, respectively. In some embodiments, arm 202a and/or arm 202b may be formed as a single integral piece. In other embodiments, arm 202a and/or arm 202b may be formed of a plurality of components (see, for example, FIGS. 6A-6D). In exemplary embodiments, at least a portion of arm 202a and/or arm 202b may be formed of flexible material, allowing at least some form of bending or flexing of arm 202a and/or arm 202b.

In various embodiments, first support element 210 may be associated with a magnet 260 (for example, a first magnet or fixed magnet). For instance, first (or fixed) magnet 260 may be affixed to or otherwise fixedly engaged with a portion of first support element 210 via an adhesive, fastener, enclosure within a cavity, and/or the like. In exemplary embodiments, magnetically-actuated clamping assembly 206 may include an actuator 230 associated with a magnet 262 (for example, a second magnet or actuator magnet). For instance, second (or actuator) magnet 262 may be affixed to or otherwise fixedly engaged with a portion of actuator 230 via an adhesive, fastener, enclosure within a cavity, and/or the like. In various embodiments, actuator 230 may be arranged within or otherwise associated with second support element 240.

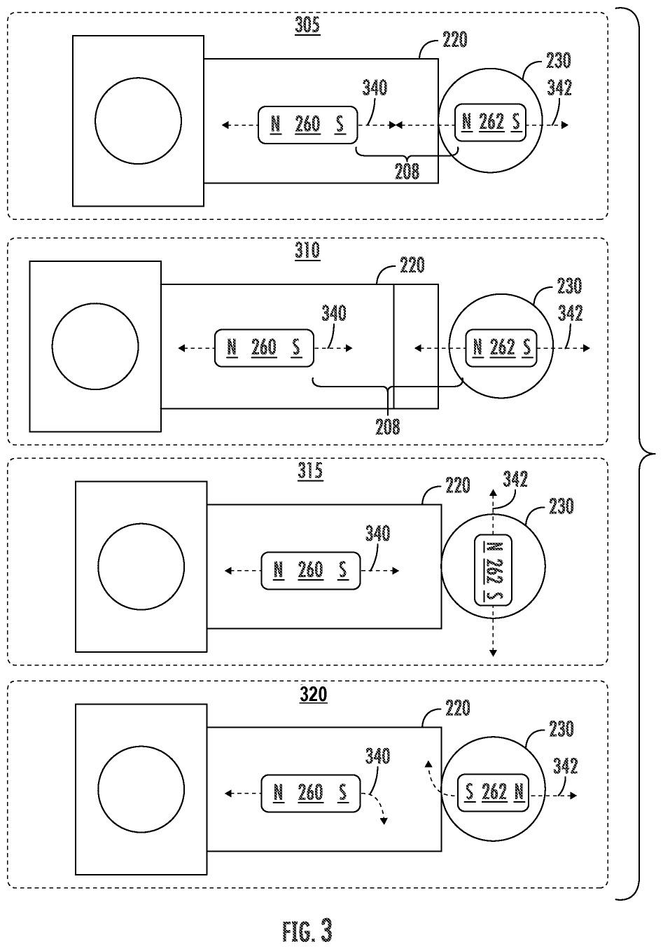

In some embodiments, actuator 230 may be configured to move to various positions to change the direction of the magnetic field associated with magnet 262. For example, actuator 230 may be configured to rotate clockwise and/or counterclockwise to change the direction of the magnetic field of magnet 262 (see, for example, FIGS. 3 and 5). In various embodiments, actuator 230 may be moved to at least one engaged position to align the magnetic field of magnet 262 with the magnetic field of magnet 260. In some embodiments, actuator 230 may be moved to at least one disengaged position to misalign the magnetic fields of magnets 260 and 262. In some embodiments, if the magnetic fields of magnet 260 and magnet 262 are aligned, and a distance 208 between magnet 260 and magnet 262 (or the magnetic fields thereof) is within a threshold distance, the forces or intensities of the resulting combined magnetic field within magnetically-actuated clamping assembly 206 may generate a magnetic clamping force.

The magnetic clamping force may be a linear force within magnetically-actuated clamping assembly 206 operative to place magnetically-actuated clamping assembly 206 in an active state in which first support element 210 and second support element 240 are rigidly merged to clamp arms 202a and 202b around femur 250. For example, the magnetic fields of magnets 260 and 262 may be aligned to combine magnetic field forces to form a magnetic field within magnetically-actuated clamping assembly which generates linear magnetic clamping force along longitudinal axis 256.

In various embodiments, when the magnetic fields of magnet 260 and magnet 262 are misaligned (or not sufficiently aligned) and/or magnet 260 and magnet 262 are not within the threshold distance (regardless of whether the magnetic fields of magnet 260 and magnet 262 are aligned), the strength of the combined magnetic fields may be eliminated or reduced below a level required to generate the magnetic clamping force. In the absence of the magnetic clamping force, magnetically-actuated clamping assembly 206 may be placed in an inactive state (and arms 202a and 202b may be moved in a releasing direction 254).

Accordingly, in some embodiments, magnetically-actuated clamping assembly 206 may implement a two-step activation process requiring both alignment of magnetic fields (for instance, of a fixed magnet and an actuator magnet) and proximity of magnets (or magnetic fields) within a threshold distance to provide a combined magnetic field strong enough to generate the magnetic clamping force to place magnetically-actuated clamping assembly 206 in the active state. In other embodiments, activation may be a single-step process, for example, requiring one of alignment of magnetic fields (for instance, for fixed-position magnets located within the threshold distance) or proximity of magnets (for instance, with pre-aligned magnetic fields). De-activation of magnetically-actuated clamping assembly 206 may require only one of misalignment of magnetic fields or movement of magnets 260 and 262 outside of the threshold distance.

In some embodiments, second magnet 260 may be arranged in or otherwise associated with an adjuster 220. In various embodiments, adjuster 220 may be slidably arranged within first support element 210 and configured to move linearly in various positions away from and toward second support element 240 along longitudinal axis 256. In various embodiments, adjuster 220 may operate to change distance 208 between first magnet 260 and second magnet 262 (or magnetic fields thereof). For example, adjuster 220 may operate to move to at least one engaged position in which magnets 260 and 262 (or magnetic fields thereof) are within the threshold distance and to at least one disengaged position in which magnets 260 and 262 (or magnetic fields thereof) are outside of the threshold distance.

The magnetic clamping force (magnetic field, magnetic field intensity, magnetic induction, magnetic flux density, B, etc.) generated when magnetically-actuated clamping assembly 206 is in the active state may be sufficient to rigidly affix magnetically-actuated mounting apparatus 205 to femur 250, for instance, without risking damage to femur 250 (for example, a fracture) and/or other anatomy, such as surrounding tissues, circulatory system anatomy, and/or the like. When magnetically-actuated mounting apparatus 205 is rigidly affixed to femur 250, magnetically-actuated mounting apparatus 205 and components thereof (for example, arms 202a and 202b, first support element 210, second support element 240, etc.) may have limited or even no freedom of movement, either rotationally or axially.

In some embodiments, the magnetic clamping force may be adjustable, for example, via positioning of actuator 230 and/or adjuster 220. For instance, actuator 230 may be placed at various positions and/or adjuster 220 may be placed at various distances 208 to achieve certain magnetic field intensities. In some embodiments, movement of actuator 230 and/or adjuster 220 may have stops, pre-defined positions, and/or the like indicating magnetic field intensity or a similar measure of the magnetic clamping force.

In some embodiments, magnetic clamping force may be about 1 Gauss (G), about 10 G, about 100 G, about 500 G, about 1000 G, about 1500 G, about 2000 G, about 5000 G, about 1 Tesla, about 5 Tesla, about 10 Tesla, about 100 Tesla, and any value or range between any two of these values (including endpoints). In some embodiments, the required magnetic clamping force may be determined based on various patient factors, such as characteristics of the portion of the human anatomy (for instance, an outer dimension of the femur), patient age, and/or the like. In various embodiments, the selection of magnets 260 and/or 262 and/or the positioning of actuator 230 and/or adjuster may be determined based on a desired magnetic clamping force value and/or range for a particular patient anatomy. In some embodiments, a CAS or similar system may automatically determine a desired magnetic clamping force, which may be communicated to an operator and/or used by robotic/automated tools to control elements for configuring the magnetic clamping force or other magnetically-actuated mounting apparatus 205 components based on patient characteristics.

Referring to FIG. 3, therein is depicted examples of active and inactive configurations of an embodiment of a magnetically-actuated clamping assembly of a magnetically-actuated mounting apparatus in accordance with features of the present disclosure. In configuration 305, actuator 230 has been moved to an engaged position, aligning magnetic field 342 with magnetic field 340 (for example, a first or fixed magnetic field). Configuration 305 depicts an active state in which magnetic field 340 of first magnet 260 is aligned with magnetic field 342 (for example, a second or actuator magnetic field) of second magnet 262, and distance 208 between first magnet 260 and second magnet is within the distance threshold. In configuration 305, combined magnetic fields 340 and 342 generate the magnetic clamping force within magnetically-actuated clamping assembly 206.

In some embodiments, first magnet 260 and second magnet 262 are magnetic dipoles with south (S) and north (N) poles. In general, magnetic fields 340 and 342 are aligned when the opposite poles of their respective magnets are facing each other. In various embodiments, in order to be aligned, magnetic fields 340 and 342 do not necessarily need to be directly facing each other (for example, the south pole of magnet 260 pointing directly at the north pole of magnet 262; a 0 incidence angle between magnetic fields 340 and 342; etc.). For example, magnetic fields 340 and 342 may be aligned even if magnets 260 and 262 (and, therefore, magnetic fields 340 and 342) are misaligned within a misalignment threshold (for example, a 10 degree incidence angle). In some embodiments, the misalignment threshold may be about 2 degrees, about 5 degrees, about 10 degrees, about 15 degrees, about 30 degrees, about 45 degrees, and any value or range between any two of these values (including endpoints). Embodiments are not limited in this context.

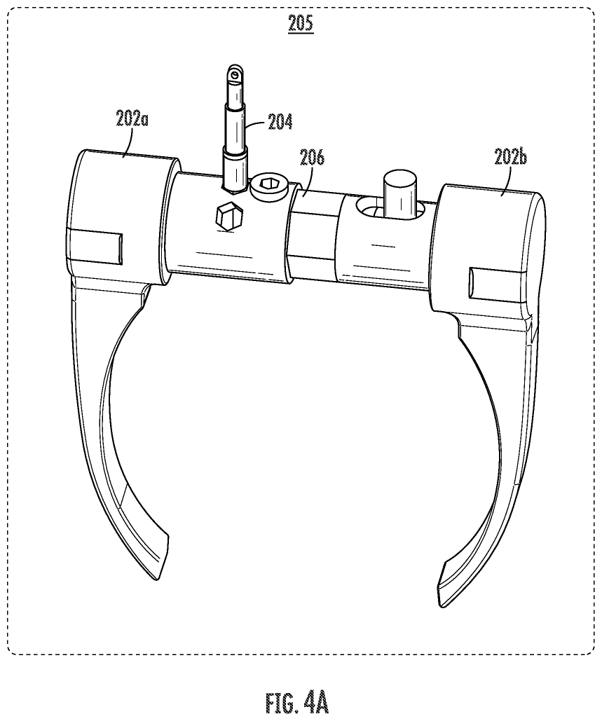

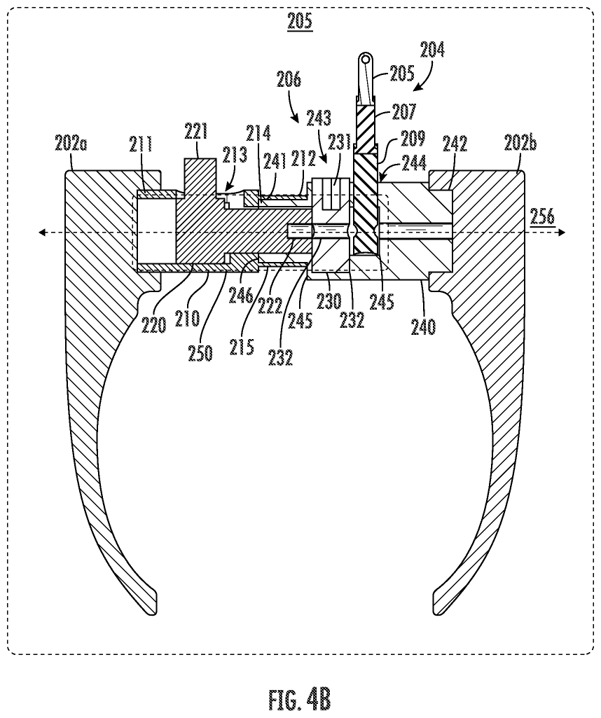

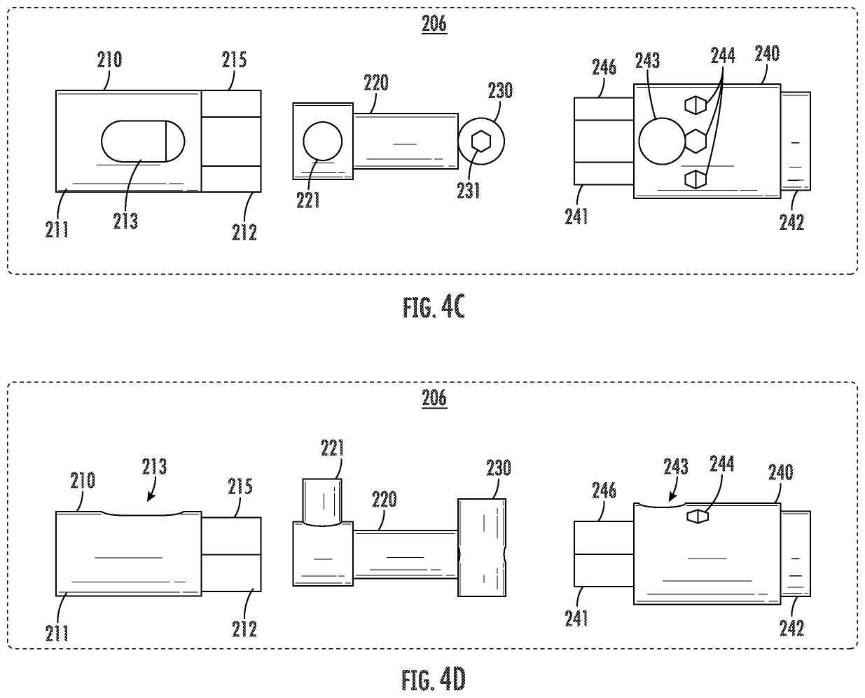

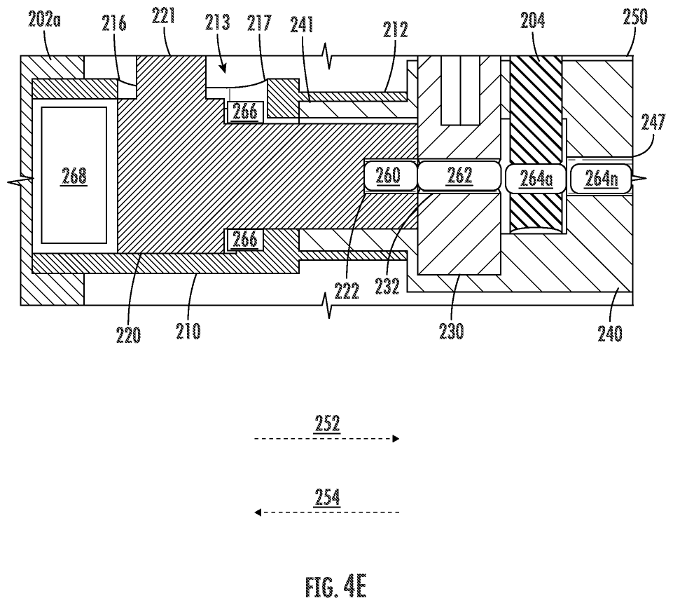

In general, the distance threshold may be any distance within which aligned magnetic fields 340 and 342 may produce a combined magnetic force sufficient to produce the magnetic clamping force, thereby placing magnetically-actuated clamping assembly 206 in an active state. For example, the distance threshold may be about 0.0 mm, about 0.05 mm, about 0.1 mm, about 0.25 millimeters (mm), about 0.5 mm, about 1.0 mm, about 2.0 mm, about 5.0 mm, about 1.0 cm, and any value or range between any two of these values (including endpoints). In various embodiments, the distance threshold may be a value that is a distance when the magnetic force overcomes a spring force (for example, spring(s) 266 of FIG. 4E) as a user brings arms 202a and 202b together. In some embodiments, distance 208 may be a constant distance within the threshold distance (for example, magnets 260 and 262 are fixed in a constant distance relative to each other) such that the magnetic clamping force is generated responsive to alignment of magnetic fields 340 and 342. For example, first magnet 260 may be arranged in first support element 210 in a fixed location instead of within adjuster 220 in an adjustable position.

Configuration 310 depicts an inactive state due to distance 208 being greater than the threshold distance. For example, adjuster 220 may be moved away from actuator 230 such that distance 208 between first magnet 260 and second magnet 262 is greater than the threshold distance. In configuration 310, actuator 230 is in the engaged position, however, the magnetic clamping force is not generated because distance 208 is too great to produce a combined magnetic field sufficient to produce the magnetic clamping force. For example, in configuration 310, magnetic fields 340 and 342 may be aligned to produce a combined magnetic field that is too weak to generate the magnetic clamping force. In another example, in configuration 310, distance 208 may be too large to allow for the combination of magnetic fields 340 and 342. In the absence of the magnetic clamping force, magnetically-actuated clamping assembly 206 is in the inactive state (for instance, an operator may manually move arms 202a and 202b in a releasing direction).

In configurations 315 and 320, magnets 260 and 262 are within the threshold distance, however, actuator 230 is in different disengaged positions in which magnetic fields 340 and 342 are misaligned. As a result of misalignment of magnetic fields 340 and 342 in configurations 315 and 320, a sufficient combined magnetic field is not generated between magnetic fields 340 and 342 to generate the magnetic clamping force. In the absence of the magnetic clamping force, magnetically-actuated clamping assembly 206 is in the inactive state.

In configuration 315, actuator 230 has been rotated to a disengaged position in which magnetic fields 340 and 342 are perpendicular to each other (for example, 90o incidence angle). In configuration 320, actuator 230 has been rotated to a disengaged position in which the same poles of magnets 260 and 262 are facing each other such that magnetic fields 340 and 342 are repelling each other. In various embodiments, in configuration 320, first support element 210 and second support element 240 may be pushed apart by the repelling force of magnetic fields 340 and 342.

Although two magnets 260 and 262 are depicted in FIG. 2, some embodiments may include more or less magnets (see, for example, FIG. 4E for an embodiment with more than two magnets). For instance, in some embodiments, clamp assembly 206 may only include one magnet, for example, an actuator magnet (such as magnet 262). In such an embodiment, second magnet 262 may be attracted to materials in first support element 210 such that first magnet 260 is not necessary to achieve the magnetic clamping force to place magnetically-actuated clamping assembly 206 in the active state. For example, second magnet 262 may be affixed or otherwise arranged in a cavity of actuator 230 having exposed ends. Actuator may have side walls or other elements that may block magnetic field 342 of magnet 262, for example, from attracting or being attracted to first support element 210 through the side walls. In order to place magnetically-actuated clamping assembly 206 in the active state, actuator 230 may be moved to an engaged position in which second magnet 262 is exposed to first support element 210 (for example, an exposed end of the cavity housing second magnet 262 is facing first support element 210 such that magnetic field 342 may attract or be attracted to first support element 210). Conversely, in order to place magnetically-actuated clamping assembly 206 in an inactive state, actuator 230 may be moved to a disengaged position in which magnetic field 342 of second magnet 262 is blocked from first support element 210. Embodiments are not limited in this context.

Referring to FIG. 2, when clamp assembly 206 is in the active state, the magnetic clamping force between magnets 260 and 262 may force first support element 210 and second support element 240 (and, therefore, arms 202a and 202b) to move or hold in clamping direction 252 toward femur 250 to rigidly affix magnetically-actuated mounting apparatus 205 to femur 250. For example, the magnetic clamping force may cause first support element 210 and second support element 240 to rigidly merge.

In the active state, magnetically-actuated mounting apparatus 205 may be affixed to femur 250 to prevent movement of magnetically-actuated mounting apparatus 205 along and/or about femur 250. For instance, in the active state, the rigid attachment may prevent magnetically-actuated mounting apparatus 206 and/or components thereof (for instance, arms 202a and 202b, first support element 210, and/or second support element 240) from moving axially along femur (for instance, along longitudinal axis 256), in a direction into or out of the page of FIG. 2, rotationally (for instance, to rotate about a longitudinal axis 256), and/or move in a releasing direction 254.

When magnetically-actuated mounting apparatus 205 is in the inactive state, magnetically-actuated mounting apparatus and components thereof (for instance, arms 202a and 202b, first support element 210, and/or second support element 240) may have freedom of movement. For example, arms 202a and 202b may encircle or are otherwise be arranged around femur 250, but magnetically-actuated clamping assembly 206 is not providing a magnetic clamping force to rigidly affix magnetically-actuated mounting apparatus 205 (and therefore, arms 202a and 202b) to femur 250. In the inactive state, magnetically-actuated mounting apparatus 205 may be removed from femur 250. In the inactive state, magnetically-actuated mounting apparatus 205 and/or components thereof (for instance, arms 202a and 202b, first support element 210, and/or second support element 240) may be moved longitudinally and/or rotationally about femur 250, for example, responsive to a manual force by an operator. Support elements 210 and 240 and/or arms 202a and 202b may be moved in a releasing direction 254 when magnetically-actuated mounting apparatus 205 is in the inactive state.

Although actuator 230 and second magnet 262 have been associated with second support element 240 and adjuster 220 and fixed magnet 260 have been associated with first support element 210, embodiments are not so limited, as actuator 230, second magnet 262, adjuster 220, and fixed magnet 260 may be associated with either first support element 210 or second support element 240 depending on the particular configuration of the embodiment. In addition, although first magnet 260 has been referred to as a fixed magnet, such reference is to simplify the description as both first magnet 260 and second magnet 262 may both be fixed or may move rotationally, axially, and/or the like according to various embodiments.

FIG. 4A shows a perspective view of an example of an embodiment of a magnetically-actuated mounting apparatus in accordance with features of the present disclosure. As shown in FIG. 4A, magnetically-actuated mounting apparatus 205 may include arms 202a and 202b attached to opposite ends of magnetically-actuated clamping assembly 206. Medical aid device 204 may be coupled to a portion of magnetically-actuated clamping assembly 206.

FIG. 4B shows a cross-sectional side view of the magnetically-actuated mounting apparatus shown in FIG. 4A. As shown in FIG. 4B, magnetically-actuated clamping assembly 206 may include a support element 210 in the form of an indexer (for example, a first indexer) having a (first) distal end 211 and a (first) proximal end 212. In some embodiments, first indexer 210 may include a top opening 213 and/or an internal shoulder 214. Arm 202a may be coupled to magnetically-actuated clamping assembly 206 via distal end 211 of first indexer 210. Magnetically-actuated clamping assembly 206 may include support element 240 in the form of an indexer (for example, a second indexer) having a (second) proximal end 241 and a (second) distal end 242. In some embodiments, second indexer 240 may include an actuator cavity 243, at least one device cavity 244, and/or an external shoulder 245. In some embodiments, arm 202b may be coupled to magnetically-actuated clamping assembly 206 via distal end 242 of second indexer 240. In various embodiments, medical aid device 204 may be arranged within and/or affixed to second indexer 240 through device cavity 244. In some embodiments, arms 202a and 202b may be attached to their respective support elements via various methods and/or structures, such as a friction fit, fasteners, locking collars, adhesives, and/or the like.

In some embodiments, first indexer 210 and second indexer 240 may directly interface with each other. In some embodiments, at least a portion of second indexer 240 may be arranged within first indexer 210 (or vice versa). For example, first indexer 210 may include a female portion 215, for instance, defined from internal shoulder 214 to proximal end 212, and second indexer may include a male portion 246, for instance, defined from external shoulder 245 to proximal end 241. Female portion 215 of first indexer 210 may be configured to receive male portion 246 of second indexer 240. The interfacing between first indexer 210 and second indexer 240 via corresponding male and female portions 215 and 246 may operate to, inter alia, provide rigidity to magnetically-actuated clamping assembly 206 (for example, limiting or preventing movement or flexing of magnetically-actuated clamping assembly 206 in longitudinal and transverse directions), while allowing first indexer 210 and second indexer 240 to move with respect to each other in clamping direction 252 and/or releasing direction 254. In some embodiments, internal shoulder 214 may prevent further movement of second indexer 240 in clamping direction. In various embodiments, one or both of first indexer 210 and second indexer 240 may include a catch, flange, protrusion, or other element configured to hinder or prevent movement of second indexer 240 away from first indexer 210 beyond a certain point (for example, proximal end 212), and/or vice versa. In exemplary embodiments, first indexer 210 and/or second indexer 240 may rotate about longitudinal axis 256, for example, when magnetically-actuated clamping assembly 206 is in the inactive state. In various embodiments, first indexer 210 and/or second indexer 240 may be made of a rigid material configured to prevent flexing or bending of first indexer 210 and/or second indexer 240. In some embodiments, one or both of first indexer 210 and/or second indexer 240 may be made of flexible material configured to allow flexing or bending of first indexer 210 and/or second indexer 240.

In some embodiments, actuator 230 may be arranged within actuator cavity 243 of second indexer 240. Actuator 230 may be rotatable, for example, about an axis perpendicular (or transverse) to longitudinal axis 256. In various embodiments, actuator 230 may rotate any number of degrees in any direction (for instance, both clockwise and counterclockwise). In various embodiments, actuator 230 may move between at least one engaged position (for instance, aligning magnetic fields) and at least one disengaged position (for instance, misaligning magnetic fields). In other embodiments, actuator 230 may be limited to rotating a certain number of degrees and/or in a certain direction. For example, actuator 230 may be limited to movement between two positions: 90° in one direction (for example, clockwise) and 90° in the opposite direction (for example, counterclockwise). In some embodiments, actuator 230 may be configured to rotate to various positions or stops, such as a stop every 90° of rotation. Embodiments are not limited in this context.

Actuator 230 may include a magnet cavity 232 configured to have a magnet arranged therein (for example, second or actuator magnet 262 as shown in FIGS. 1, 3, and 5). The magnet may be rigidly affixed to actuator 230, for example, via an adhesive, fastener, holding compartment, and/or the like. In an exemplary embodiment, as actuator 230 is rotated, second magnet 262 may correspondingly rotate. In various embodiments, actuator 230 may include a drive 231 configured to receive a tool (for example, a hex or Allen driver, screwdriver, and/or the like) for manually rotating actuator 230. As shown in FIG. 3, rotation of actuator 230 may operate to change the direction of magnetic field 342 associated with second magnet 262 arranged within actuator 230.

In various embodiments, first indexer 210 may include a magnet arranged therein (for example, first or fixed magnet 260 as shown in FIGS. 1, 3, and 5). In some embodiments, the magnet may be arranged within a magnet cavity 222 of an adjuster 220 slidably arranged within first indexer 210. In various embodiments, adjuster 220 may operate to slide within first indexer 210 in clamping direction 252 toward second indexer 240 (and actuator 230) and in releasing direction 254 away from second indexer 240 (and actuator 230). In some embodiments, adjuster 220 may include a post 221 protruding through top opening 213 of first indexer 210. Post 221 may be manipulated manually and/or via a tool for an operator and/or robotic device to move adjuster 220. In some embodiments, adjuster 220 may move between an engaged position (for instance, magnets or magnetic fields within the threshold distance) and a disengaged position (for instance, magnets or magnetic fields outside of the threshold distance).

In the example embodiments shown in FIGS. 4A and 4B, medical aid device 204 is a telescoping device, for example, configured to hold a communication array or other element for a navigated surgical procedure. Medical aid device 204 may include multiple portions, including, without limitation, a top portion 205, a middle portion 207, and a bottom portion 209. In various embodiments, top portion 205 may include elements configured to mount a tracking array or other element to medical aid device 204. In some embodiments, middle portion 207 may be configured as a telescoping element configured to increase/decrease the height of top portion 205. In exemplary embodiments, bottom portion 209 may be configured to fit within at least one device cavity 244.

In some embodiments, at least one portion of medical aid device 204, such as top portion 205, may be configured to move to change a position of medical aid device 204, such as rotating, pivoting, and/or the like. In this manner, a position of medical aid device 204 and/or a device attached thereto may be adjusted with respect to magnetically-actuated mounting apparatus 205 and/or femur 250. Medical aid device 204 may be affixed to second indexer 240 via various methods, including a snap fit, a friction fit, a magnetic force, a mechanical force, a pneumatic force, a spring force, and/or the like. In some embodiments, a mechanical lock (for instance, a collared lock, a tapered lock, and/or the like) may be used to affix medical aid device 204 to magnetically-actuated clamping assembly 206. Embodiments are not limited in this context.

FIG. 4C shows an exploded top view of the magnetically-actuated clamping assembly depicted in FIG. 4A. FIG. 4D shows an exploded side view the magnetically-actuated clamping assembly depicted in FIG. 4A. As shown in FIG. 4C, second indexer 240 may include a plurality of medical aid cavities 244. Accordingly, in some embodiments, magnetically-actuated clamping assembly 206 may hold a plurality of medical aid devices 204 and/or may hold a medical aid device 204 in various positions.

In some embodiments, medical aid cavities 244 may have a pattern that matches a pattern of a corresponding portion of medical aid device 204. For example, a medical aid cavity may have a hexagonal pattern that matches a hexagonal pattern of a bottom portion 209 of a telescoping tracking array medical aid device 204. The hexagonal pattern of the base of the array and the multiple hexagonal hole pattern of medical aid cavity 244, for example, embossed in the second indexer 240, may allow for variable positioning of an array. Paired with a telescopic shaft connection, such embodiments may allow for an optimized range of detection by a navigation device, such as a camera.

FIG. 4E depicts a close-up view of area 250 of magnetically-actuated mounting apparatus 205 shown in FIG. 4A. In some embodiments, at least one spring 266 may be arranged within first indexer 210. In various embodiments, spring 266 may be biased to push adjuster 220 in releasing direction 254 away from second indexer 240 and actuator 230. In exemplary embodiments, the magnetic clamping force between first magnet 260 and second magnet 262 when they are aligned and within the threshold distance may be greater than the spring force generated by spring 266. Accordingly, when magnetically-actuated clamping assembly 206 is in the active state, the spring force of spring 266 may be overcome by the magnetic clamping force such that adjuster 220 is forced in clamping direction 252 (see, for example, FIG. 5).

Opening 213 may include a distal surface 216 and a proximal surface 217. In some embodiments, post 221 may interface with distal surface 216 to push, bias, force, hold, and/or the like first indexer 210 in releasing direction 254 away from second indexer 240. For example, when magnetically-actuated clamping assembly 206 is in the inactive state, spring 266 may push or bias adjuster 266 in releasing direction 254 such that post 221 engages distal surface 216 and pushes or biases first indexer 210 in releasing direction 254. In another example, when magnetically-actuated clamping assembly 206 is in the active state, the magnetic clamping force may push adjuster 266 in clamping direction 252 such that post 221 engages proximal surface 217 and pushes, biases, forces, holds, and/or the like first indexer 210 in clamping direction 252.

In various embodiments, post 221 does not engage distal surface 216 and/or proximal surface 217. For example, when magnetically-actuated clamping assembly 206 is in the active state, adjuster 220 may be held against actuator 230 by the magnetic clamping force while there is space between post 221 and proximal surface 217. In such an embodiment, first indexer 210 may move the distance of the space between post 221 and proximal surface 217 in releasing direction 254, for example, to provide some flexibility of movement when magnetically-actuated clamping assembly 206 is in the active state. In another example, pushing by post 221 against distal surface 216 may not cause first indexer to move 210, for example, in releasing direction 254.

As shown in FIG. 4E, auxiliary magnets 264a-n may be associated with various elements of magnetically-actuated clamping assembly 206. For example, at least one magnet 264a may be attached to or otherwise associated with medical aid device 204. For example, magnet 264a may be coupled to the shaft of a tracking array. In some embodiments, magnet 264a on the shaft of tracking array 204 may be introduced into the magnetic fields of magnets 260 and/or 262 and may maintain the position of medical aid device 204 as magnet 264a is aligned to the other magnetic field(s) of magnetically-actuated clamping assembly 206. In some embodiments, a tracking array medical aid device 204 may be introduced and removed normal to the magnetic field(s) of magnetically-actuated clamping assembly such that high acceleration due to magnetic field forces may be avoided, thereby, allowing ease of assembly/disassembly. In some embodiments, magnet 264a may operate to hold medical aid device 204 within second indexer 240.

In another example, second indexer 240 may include a cavity 247 configured to store at least one magnet 264n therein. In various embodiments, the magnetic fields of auxiliary magnets 264a-n may be added to the magnetic fields associated with magnets 260 and 262 (for instance, magnetic fields 340 and 342, respectively) to generate the magnetic clamping force. In other embodiments, the magnetic fields of one or more of auxiliary magnets 264a-n may be isolated from the magnetic fields associated with magnets 260 and 262 so that they do not add (or materially add) to the magnetic clamping force.

In various embodiments, an auxiliary force element 268 may be arranged within magnetically-actuated clamping assembly 206. Although in FIG. 4E, auxiliary force element 268 is shown arranged in first indexer 210, embodiments are not so limited as one or more auxiliary force elements 268 may be arranged in other components of magnetically-actuated clamping assembly 206. Auxiliary force element 268 may include a spring, a hydraulic device, a mechanical device, a solenoid device, an electromagnetic device, and/or the like configured to bias, push, pull, rotate, hold, or otherwise manipulate one or more portions of magnetically-actuated clamping assembly 206.

For example, auxiliary force element 268 may include a device to push and/or hold adjuster 220 in clamping direction 252 (for instance, engaged position) and/or to pull adjuster 220 in a releasing direction 254 (for instance, disengaged position). In another example, auxiliary force element 268 may include a device arranged in contact with actuator 230 to move actuator 230 (for example, to move between engaged and disengaged positions). In a further example, auxiliary force element 268 may operate to manipulate medical aid device 204, magnets 260 and 262, and/or auxiliary magnets 264a-n. In an additional example, auxiliary force element 268 may operate as an additional clamping force, for example, alone or in combination with the magnetic clamping force to move or hold magnetically-actuated clamping assembly 206 in the active state. In some embodiments, auxiliary force element 268 may be, may include, or may be operably coupled with a remotely controlled logic device. For example, auxiliary force element 268 may operate to control components of magnetically-actuated clamping assembly 206, such as indexer 230, or the distance, position, polarity, strength, or other feature of magnets 260 or 262 to control the clamping status of magnetically-actuated clamping assembly 206. In this manner, some or all manipulations of components of magnetically-actuated clamping assembly 206 may be remotely controlled via a computer and/or an operator using a computing device. Embodiments are not limited in this context.

In some embodiments, one or more auxiliary locking elements (not shown) may be used to lock magnetically-actuated clamping assembly 206 in the active state. Non-limiting examples of auxiliary locking elements may include a threaded collar. For example, an auxiliary locking element may be secured to magnetically-actuated mounting apparatus 205 when magnetically-actuated clamping assembly 206 has been placed in the active state to assist in rigidly holding magnetically-actuated mounting apparatus 205 to femur 250.

FIG. 5 shows cross-sectional side views of active and inactive configurations of a magnetically-actuated clamping assembly in accordance with features of the present disclosure. Configuration 505 shows an inactive state of magnetically-actuated clamping assembly 206. In configuration 505, actuator 230 is positioned in the engaged position in which first magnetic field 340 and second magnetic field 342 are aligned. Distance 208 is greater than the threshold distance such that a magnetic clamping force is not generated to place magnetically-actuated clamping assembly 206 in the active state. In configuration 505, spring 266 is in an extended (or uncompressed, partially extended, or partially uncompressed) position, thereby biasing adjuster 220 in releasing direction 254. In the example embodiment of FIG. 5, there may be a gap between first indexer 210 and second indexer 240 when magnetically-actuated clamping assembly 206 is in the inactive state (for instance, first indexer 210 and/or second indexer 240 are able to move away from each other in respective releasing directions 254).

In configuration 510, adjuster 220 has been moved to the engaged position in which distance 208 is less than the distance threshold, thereby causing a sufficient magnetic clamping force to place magnetically-actuated clamping assembly 206 in the active state. In the example embodiment of FIG. 5, first indexer 210 and second indexer 240 may interface when magnetically-actuated clamping assembly 206 is in the active state (for example, proximal end 212 of first indexer 210 may push against or otherwise engage a corresponding surface of second indexer 240, such as external shoulder 245, and/or proximal end 241 of second indexer 240 may push against or otherwise engage a corresponding surface of first indexer 210, such as internal shoulder 215). Embodiments are not limited in this context.

In configuration 515, actuator 230 has been moved to the disengaged position such that magnetic fields 340 and 342 (directed into/out of the page of FIG. 5) are misaligned. As a result, magnetic fields 340 and 342 do not combine in a manner that generates the magnetic clamping force to place magnetically-actuated clamping assembly 206 in the active state. In various embodiments, adjuster 220 may be pushed, biased, or otherwise manipulated in releasing direction 254 via spring 266. Accordingly, when actuator 230 is moved to the disengaged position, reducing or eliminating the magnetic clamping force, distance 208 may be increased (including placing adjuster 220 in the disengaged position).

FIGS. 6A and 6B show perspective views of an example of an embodiment of a magnetically-actuated mounting apparatus with an offset arm in accordance with features of the present disclosure. As shown in FIGS. 6A and 6B, a magnetically-actuated mounting apparatus 605 may include at least one offset arm 610 coupled to a portion of mounting assembly 206. In various embodiments, offset arm 610 may include a connector 611 having a head 612 and a shaft 613 with a pin cavity 614 arranged through shaft 613, a swivel 615 having a shaft cavity 616 arranged therethrough, a swivel pin 617, and/or a foot 618. In some embodiments, one or more portions of foot 618 may include protrusions 622 (for example, claws, teeth, projections, needles, and/or the like) configured to facilitate gripping of foot to a portion of the human body, such as femur 250 (see, for example, FIGS. 7A-D).

FIG. 6C shows a cross-sectional side view of the magnetically-actuated mounting apparatus shown in FIGS. 6A and 6B. As shown in FIG. 6C, offset arm 610 may be coupled to a support element of magnetically-actuated mounting apparatus 206, for example, head 612 of connector 611 may be coupled to second indexer 240. Although offset arm 610 is depicted as being coupled to second indexer 240, embodiments are not so limited as offset arm 610 may be coupled to other portions of magnetically-actuated clamping assembly 206, such as first indexer 210. Shaft 613 of connector 611 may be arranged through shaft cavity 616 of swivel 615. A swivel pin 617 may be arranged within pin cavity 614 of shaft 613, for example, to prevent movement of swivel 615 away from head 612. In some embodiments, swivel pin 617 may be rigidly arranged within pin cavity 614, for instance, via a friction fit, interlocking elements, cotter pin, and/or the like.

In some embodiments, swivel 615 may be rigidly coupled to shaft 613 such that swivel does not rotate about shaft 613 and/or move longitudinally along shaft 613. In other embodiments, swivel 615 may be configured to rotate about shaft 613 and/or to move longitudinally along shaft in one or more directions (for instance, toward swivel pin 617 and/or toward head 612).

In various embodiments, foot 618 may have a ball 620, for example, arranged at the top of a shaft 620 and positioned inside a corresponding socket or cup 621 of swivel 615. In this manner, foot 618 and swivel 615 may implement a ball-and-socket joint allowing multidirectional movement and rotation of foot 618. FIG. 6D shows an exploded, perspective side view of an example of an embodiment of the offset arm of the magnetically-actuated mounting apparatus shown in FIGS. 6A-6C.

As shown in FIGS. 6A-6D, some embodiments may include an offset arm 610 having sharp teeth or claws arranged on foot 618, that promotes adequate lateral fixation onto a femur or other portion of a human body. Offset arm 610 may be connected to magnetically-actuated clamping assembly 206 via a modified ball (for example, 620) and socket (for example, 621) feature that may enable multiple degrees of freedom for proper placement of magnetically-actuated mounting apparatus 205, for example, due to varying anatomic structures among patients.

FIG. 7 illustrates an embodiment of a method flow 700. Method flow 700 may be representative of some or all of the operations for using a magnetically-actuated mounting apparatus according to some embodiments herein, for example, by an operator (for example, a surgeon) and/or a logic device (for example, a CAS system). Method flow 700 may be representative of some or all of the operations of a process for placing a magnetically-actuated mounting apparatus in an active state and an inactive state according to some embodiments.

At step 710, a segment of method flow 700 including steps 712-716 for activating a magnetically-actuated mounting apparatus may be initiated. Method flow 700 may include placing the arms of a magnetically-actuated mounting apparatus around a portion of a human body at step 712. For example, magnetically-actuated mounting apparatus 205 may be installed on femur 250 such that arms 202a and 202b at least partially encircle a portion of femur 250. Although step 710 is depicted as occurring before step 712, embodiments are not so limited, as step 712 may occur prior to step 710.

At step 714, method flow 700 may include moving an actuator to an engaged position to align a magnetic field of an actuator magnet with the magnetic field of a fixed magnet. For example, actuator 230 may be moved to a position as shown in configuration 305 of FIG. 3 and/or configurations 505 and 510 of FIG. 5 such that magnetic field 342 of magnet 262 is aligned with magnetic field 340 of magnet 260. Method flow 700 may include step 716 of moving the fixed magnet within a threshold distance of the actuator magnet. For example, with reference to FIG. 5, adjuster 220 may be moved to an engaged position as shown in configuration 510 such that first (or fixed) magnet 260 is a distance 208 from second (or actuator) magnet 262 that is within the threshold distance.

At step 720, a segment of method flow 700 for de-activating a magnetically-actuated mounting apparatus may be initiated. At step 722, method flow 700 may include rotating the actuator to a disengaged position to misalign the magnetic field of the actuator magnet with the magnetic field of the fixed magnet. For example, with reference to FIG. 5, actuator 230 may be moved to a disengaged position as shown in configuration 515 such that magnetic field 340 is misaligned with magnetic field 342 (see also, configurations 315 and 320 of FIG. 3). Alternatively, at step 724, method flow 700 may include moving fixed magnet outside of a threshold distance from actuator magnet. For example, adjuster 220 may be moved in releasing direction 254 such that distance between first magnet 260 and second magnet 262 is greater than the threshold distance. In the inactive state, components of magnetically-actuated mounting apparatus 205, such as arms 202a and 202b, indexers 210 and 240, adjuster 220, and/or the like may have freedom of movement and may be moved or otherwise manipulated by an operator, such as a surgeon, and/or a robotic device.

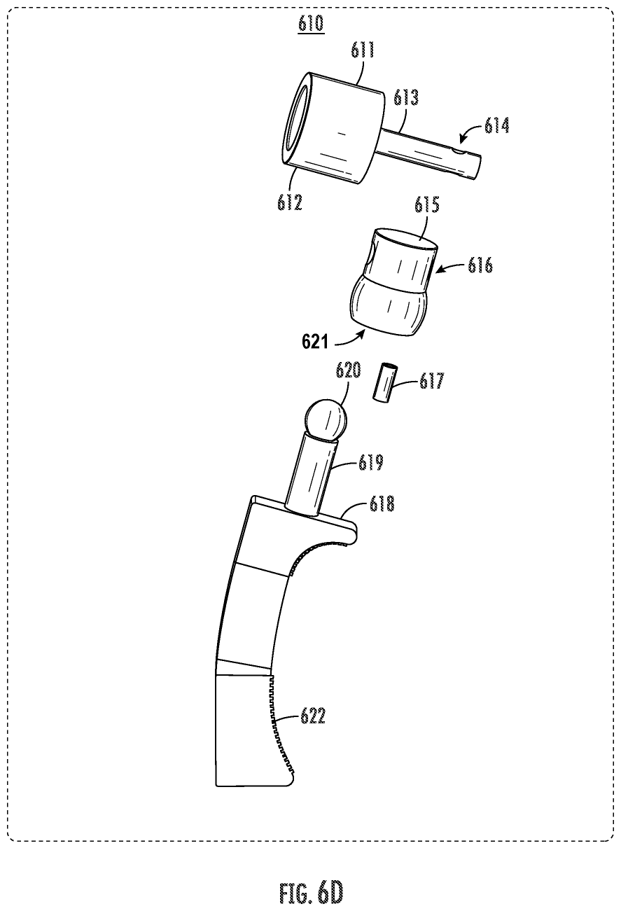

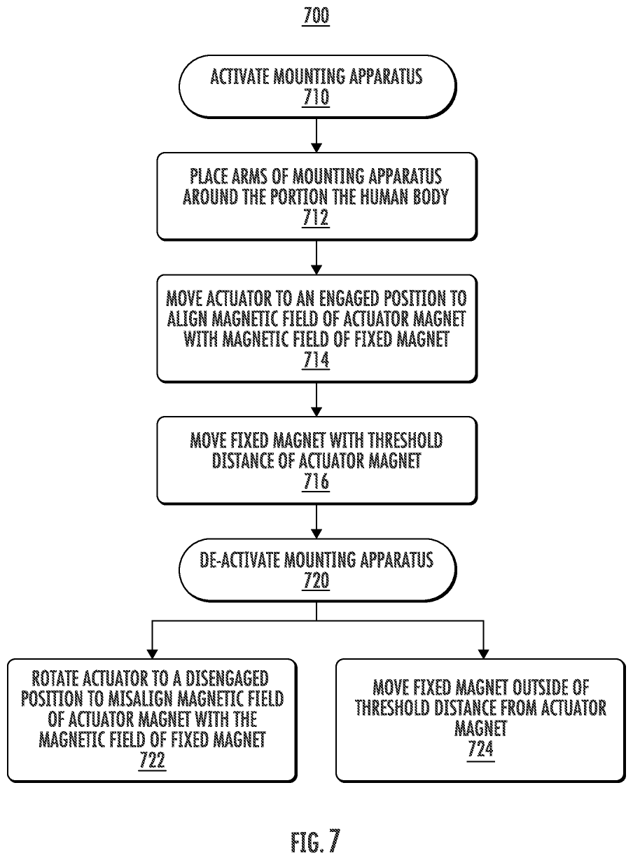

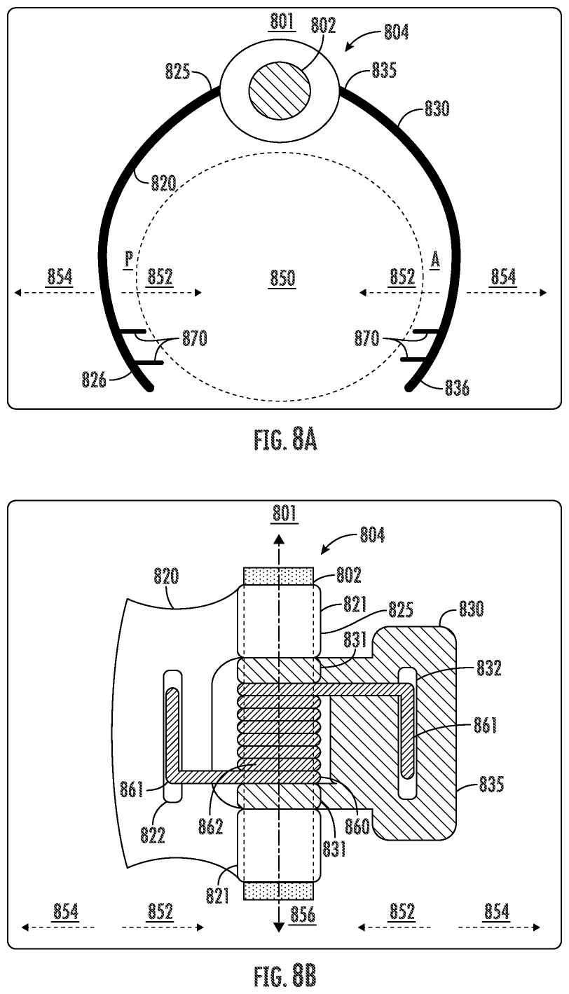

FIG. 8A shows a side view of a block diagram of an example of an embodiment of a spring-actuated mounting apparatus in accordance with features of the present disclosure. As shown in FIG. 8A, a spring-actuated mounting apparatus 801 may include a pair of opposing arms 820 and 830 configured to be arranged around a portion of a human body 850, such as a femur. Each of arms 820 and 830 may have a connection end 825 and 835, respectively, configured to be coupled to, arranged around, or otherwise engaged with spring-actuated clamping assembly 804. Arms 820 and 830 may have an engagement end 826 and 836, respectively, to engage a portion of femur 850 when spring-actuated mounting apparatus 801 is activated in a clamping position. In various embodiments, one or both of arms 820 and 830 may include protrusions 870. In some embodiments, protrusions 870 may include teeth, spikes, needles, bumps, and/or similar structures that may operate to engage (for example, dig or bite into) femur 850 and/or associated anatomical structures to further facilitate attachment of spring-actuated mounting apparatus 801 to femur 850.

In some embodiments, the configuration (for instance, shape, size, contour, and/or the like) of opposing arms 820 and 830 may be the same or substantially the same. In other embodiments, the configuration of opposing arms 820 and 830 may be different (see, for example, FIGS. 8B, 9A-9C, and 10). For example, arm 820 may be an anterior arm configured to engage an anterior (A) or substantially anterior side of femur 850. Arm 830 may be a posterior arm 830 configured to engage a posterior (P) or substantially posterior side of femur 850. In various embodiments, anterior arm 820 may be configured to engage the anterior face of the proximal femur superior to the lesser trochanter 850, and posterior arm 830 may be configured to the lesser trochanter region of femur 850 on a side opposite anterior arm 820. Embodiments are not limited in this context.

In various embodiments, spring-actuated clamping assembly 804 may include a pin 802 and a spring mechanism (not shown; see FIGS. 8B, 9A-9C, and 10). Pin 802 may be configured to extend through connection ends 825 and 835 of arms 820 and 830 such that arms 820 and 830 may rotate or pivot about pin 802 in one of a releasing direction 854 (for instance, movement of arm 820 and/or 830 away from each other) or a clamping direction 852 (for instance, movement of arm 820 and/or 830 toward each other). In various embodiments, the spring may be arranged to bias, force, hold, or otherwise compress arms 820 and 830 against femur 850 with sufficient force to maintain a rigid hold on femur 850. When spring-actuated mounting apparatus 801 is rigidly affixed to femur 850, spring-actuated mounting apparatus 801 and components thereof (for example, arms 820 and 830) may have limited or no freedom of movement, either rotationally or axially.

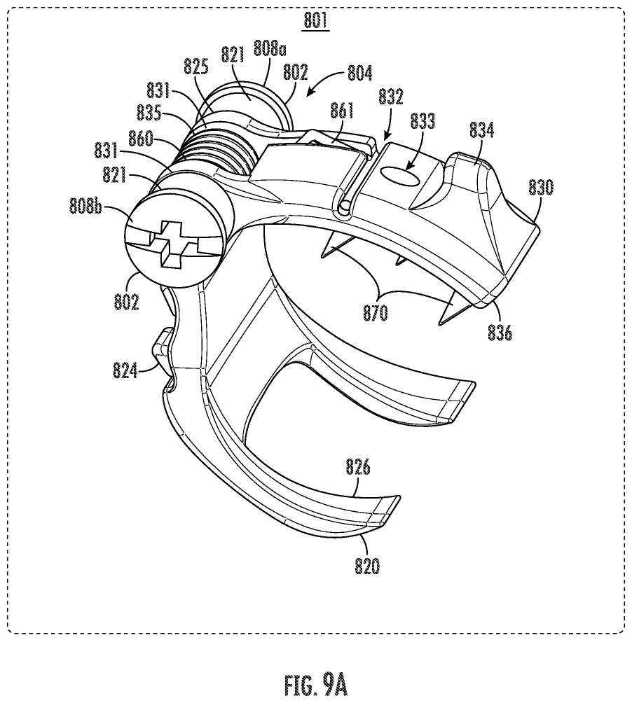

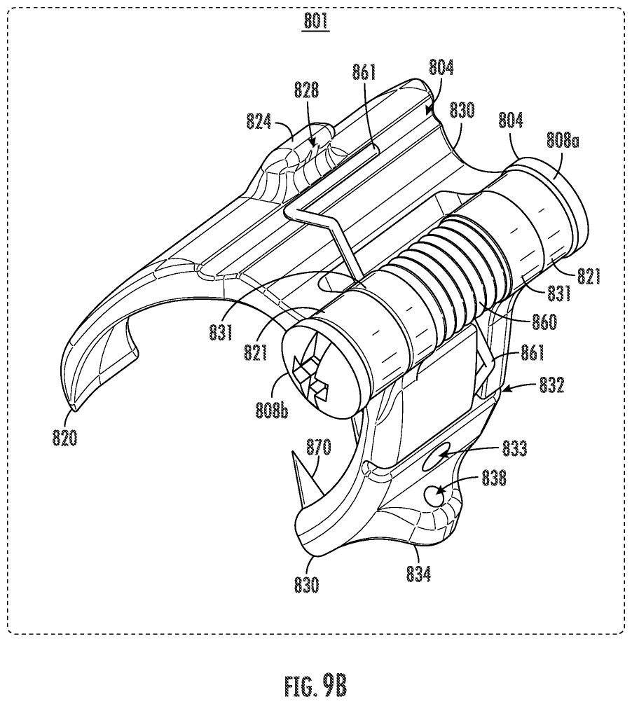

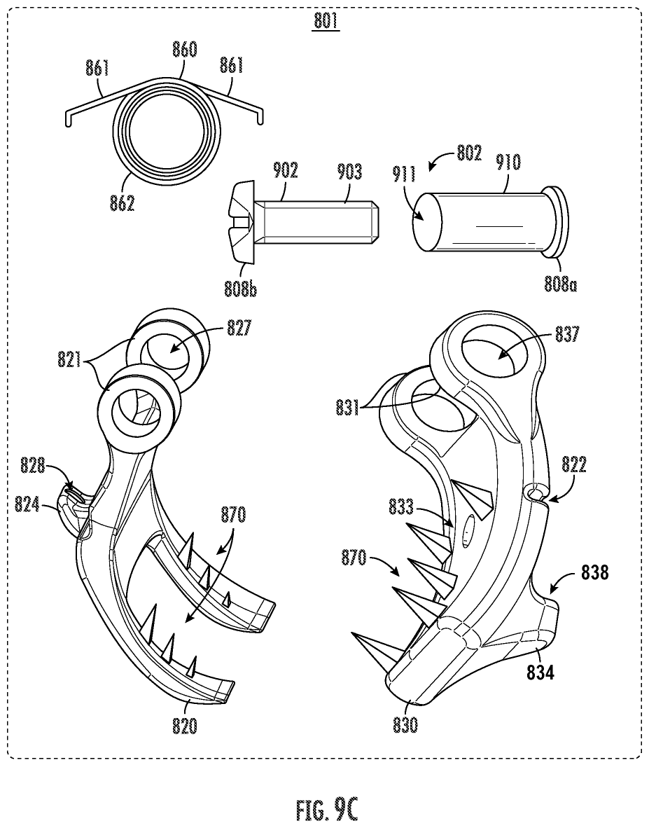

FIG. 8B shows a top view a top view of a block diagram of an example of an embodiment of a spring-actuated mounting apparatus in accordance with features of the present disclosure. As shown in FIG. 8B, arm 820 may have prongs 821 arranged at connection end 825, and arm 830 may have prongs 831 at connection end 835 (see, for example, FIG. 9C for perspective view of prongs 821 and 831). Pin 802 may extend through prongs 821 and 831 to couple spring-actuated clamping assembly 804 to arms 820 and 830 (dashed lines depict internal view of pin 802 extending through prongs 821 and 831 and spring mechanism 860 along longitudinal axis 856 of spring-actuated clamping assembly 804). Arms 820 and 830 may rotate or pivot about pin 802 at prongs 821 and 831, for example, in clamping direction 852 and/or releasing direction 854.

Spring mechanism 860 may be arranged around pin 802. In some embodiments, spring mechanism 860 may include a spring, such as a torsion spring. Although a torsion spring is used in some examples herein, embodiments are not so limited as other types of springs and spring mechanisms capable of operating according to some embodiments are contemplated in the present disclosure.

In various embodiments, at least a portion of spring 860 may operate to engage one or both of arms 820 and 830 to force or bias arms 820 and/or 830 in clamping direction 852. For example, in various embodiments, spring 860 may include a central body 862 formed of a plurality of coils. Hooks 861 (arms, extensions, protrusions, and/or the like) may extend from central body 862 that may engage arms 820 and 830. For example, in some embodiments, hooks 861 may be seated within grooves 822 and 832 of arms 820 and 830, respectively.

Spring 860 may be configured to provide a spring force sufficient to rigidly affix spring-actuated mounting apparatus 801 to femur 850, without damaging femur 850 or other associated anatomy. For example, spring 860 may be configured to provide a force of about 0.1 Newtons (N), about 0.5 N, about 1.0 N, about 2.0 N, about 5.0 N, about 10 N, about 20 N, about 30 N, about 40 N, about 50 N, about 60 N, about 70 N, about 80 N, about 90 N, about 100 N, about 150 N, about 200 N, about 850 N, about 500 N, about 1000 N, about 2000 N, and any value or range between any two of these values (including endpoints). The force generated by spring 860 may depend on various spring factors. For example, for a torsion spring, the force may depend on the number of coils, coil diameters, hook length, hook angle, and/or the like. In some embodiments, the force generated by spring 860 may be adjusted by a user before or after installation of the spring-actuated mounting apparatus at the target site, for example, by modifying one or more spring factors. In another example, spring 860 may be selected with certain spring factors in order to provide a specific amount of compression force.

FIGS. 9A and 9B show first and second perspective views of an example of an embodiment of a spring-actuated mounting apparatus in accordance with features of the present disclosure. In the embodiment of FIG. 9A, arm 820 may be a posterior arm configured to engage the lesser trochanter region of a femur (not shown), and arm 830 may be an anterior arm configured to engage the anterior face of the lesser trochanter region of femur (see, for example, FIG. 10) on a side opposite arm 820. In various embodiments, arms 820 or 830 may be bifurcated at an engagement end 826 or 836. For example, arm 820 may have a bifurcated engagement end 826 so that arm 820 may straddle the lesser trochanter region of a femur. In some embodiments, arms 820 or 830 may include protrusions 870, for example, at engagement end 826 or 836.

In various embodiments, spring-actuated clamping assembly 804 may include pin 802 and spring 860. In some embodiments, pin 802 may be or may include a barrel nut (or coupling), a binding post, post and screw fastener, a sex bolt, a Chicago screw, architectural screw, and/or the like. For example, pin 802 may include a barrel-shaped portion with a protruding cylinder or boss that is configured to receive a corresponding fastener or shaft. In some embodiments, the protruding cylinder may be internally threaded to engage a corresponding screw. FIG. 9C shows an exploded view of the spring-actuated clamping assembly shown in FIG. 9A. As shown in FIG. 9C, pin 802 may include a barrel 910 having a flange 808a (barrel flange) and a cylindrical body with cylindrical cavity 911. The inner walls of barrel 910 within cavity 911 may be threaded, for example, to receive corresponding threads 903 of screw 902. Screw 902 may have a head 808b, for example, configured to receive a tool to thread screw 902 into barrel 910.

Although a threaded screw 902 and corresponding threaded barrel 910 are used in examples herein, embodiments are not so limited, as pin 802 may be formed of various other components, such as snap fit components, friction fit components, cotter pin, single integrated component, and/or the like. Embodiments are not limited in this context.

In various embodiments, prongs 821 and 831 may each have openings 827 and 837, respectively, that may receive pin 802 (for instance, barrel 910 of pin 802). Referring to FIGS. 9A and 9C, barrel 910 may be extended through openings 827 and 837 of prongs 821 and screw 902 may be threaded into barrel 910. When screw 902 is threaded into barrel 910, screw 902 and barrel 910 may essentially form pin 802, with flange 808a and head 808b holding arms 820 and 830 in place about pin 802. Pin 802 may also extend through central body 862 of spring 860 to form spring-actuated clamping assembly 804 for arms 820 and 830.

In various embodiments, prongs 831 may be spaced so as to fit within prongs 821 (or vice versa). In some embodiments, spring-actuated mounting apparatus 801 may be formed by placing prongs 831 within prongs 821, with spring 860 arranged between prongs 821 and hooks 861 seated within grooves 822 and 832. Barrel 910 may be pushed through the tube or cylinder formed by prongs 821 and 831 and central body 862 of spring 860, and screw 902 may be threaded (or otherwise engaged) within cavity 911 of barrel 910 to form pin 802. In this manner, pin 802 may hold arms 820 and 830 together at connection ends 825 and 835, and hooks 861 of spring 860 may bias arms 820 and 830 in clamping direction.

Hooks 861 of spring may be biased in compression direction 852. Accordingly, hooks 861 may be configured to push on an outer surface of arms 820 and 830 to compress arms 820 and 830 around the anatomical structure. The inner diameter of spring 860 may be greater than the outer diameter of pin 802. In various embodiments, spring 860 may compress when hooks 861 push arms 820 and 830 in compression direction 852, causing the inner diameter of spring 860 to decrease, thereby compressing central body 862 of spring 860 around pin 802.

In some embodiments, spring-actuated mounting apparatus 801 may be in a closed or clamping state (or substantially closed or clamping state) by default due to the compression force of hooks 861 on arms 820 and 830. The spring force may create a closed bias that may assist with initial placement of spring-actuated mounting apparatus 801 at a target installation site.

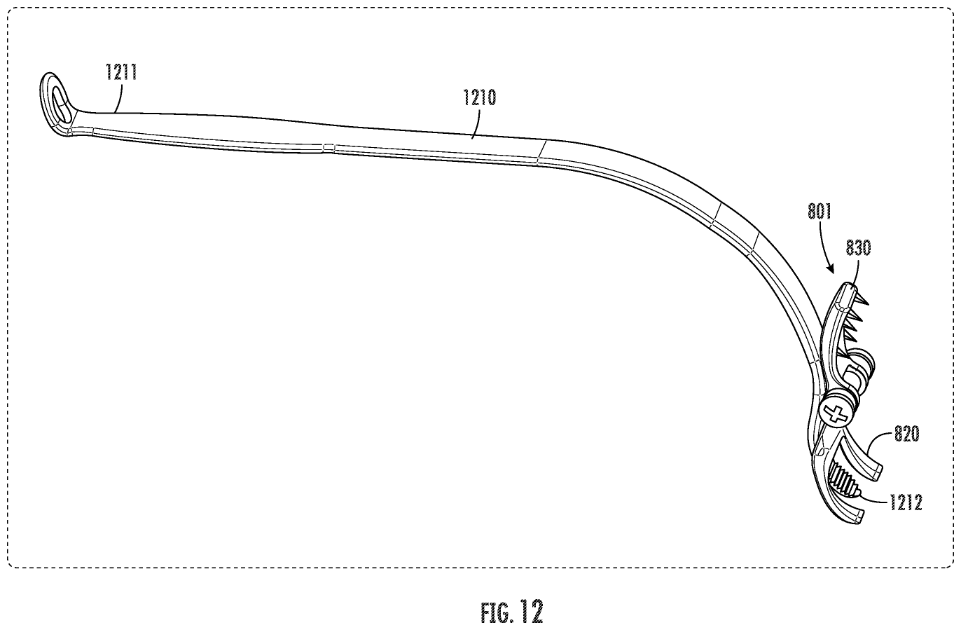

In various embodiments, spring-actuated mounting apparatus 801 may be held in an open state, for instance, in which a distance between arm 820 and 830 is wider than the target installation site to allow spring-actuated mounting apparatus 801 to be seated around the anatomical structure. For instance, a release or retractor device (not shown; see, for example, FIG. 12) may engage portions of spring-actuated mounting apparatus 801 to place spring-actuated mounting apparatus in the open state. In another instance, a holding device (not shown; for example, a band, a clip, a pin, a retractor, and/or the like) may hold spring-actuated mounting apparatus 801 in the open state, for example, by forcing open arms 820 and 830 with a greater force than the clamping force provided via hooks 861. When spring-actuated mounting apparatus 801 is arranged around the target site, the release device or holding device may be removed, thereby allowing hooks 861 to rigidly compress arms 820 and 830 around the target portion of the anatomical structure.

In some embodiments, arm 820 and/or 830 may include a fastener opening 833. In various embodiments, fastener opening 833 may be configured to receive a screw or other fastener to affix arm 820 and/or 830 to femur. For example, an anterior side of spring-actuated mounting apparatus (arm 830) may be screwed into the anterior face of the lesser trochanter region of femur. However, fastening spring-actuated mounting apparatus 801 to a femur via fastener opening 833 is not required to achieve rigid attachment of spring-actuated mounting apparatus 801 according to some embodiments as spring-actuated clamping assembly 804 may provide sufficient force to achieve rigid attachment of spring-actuated mounting apparatus 801 to a femur (or other anatomical structure).

Referring to FIGS. 9A and 9B, arms 820 and 830 may include release attachments 824 and 834, respectively. In some embodiments, release attachments 824 and 834 may be configured to receive a release device, a holding device, or other tool (see, for example, FIG. 12). In some embodiments, release attachments 824 and 834 may include cavities 828 and 838, respectively, configured to receive corresponding protrusions on a release device to allow the release device to engage spring-actuated mounting apparatus 801. In various embodiments, the release device may be used to pry, pull, or otherwise force one or both of arms 820 and 830 in releasing direction 854 to allow spring-actuated mounting apparatus 801 to be released from the femur.

A non-limiting example of a releasing device capable of being used in combination with release attachments 824 and 834 may include a Gelpi retractor, a cobra retractor, or a similar retractor component, for example, to open spring-actuated mounting apparatus 801 and then release spring-actuated mounting apparatus 801 when placed onto femur 850.

FIG. 10 shows an example of an embodiment of a spring-actuated mounting apparatus attached to a portion of a femur in accordance with features of the present disclosure. As shown in FIG. 10, spring-actuated mounting apparatus 801 may be affixed to femur 850, for example, at a medial side of femur 850. Arm 820 may be configured as a posterior arm having two claws to straddle the lesser trochanter 1012, and arm 830 may be configured as an anterior arm configured to engage the lesser trochanter.

In the embodiment depicted in FIG. 10, an auxiliary screw 1010 may be used to provide additional support for holding spring-actuated mounting apparatus 801 to femur 205. Screw 1010 may be secured into the bone for further fixation while also pulling protrusions (not shown; for example, spikes and/or teeth) on the underside of anterior arm 830 into the face of the bone. Adequately spaced and contoured claws formed from bifurcation of posterior arm 820 may slide around the medial portion of the proximal femur until they are properly placed firmly against the horn of lesser trochanter 1012.

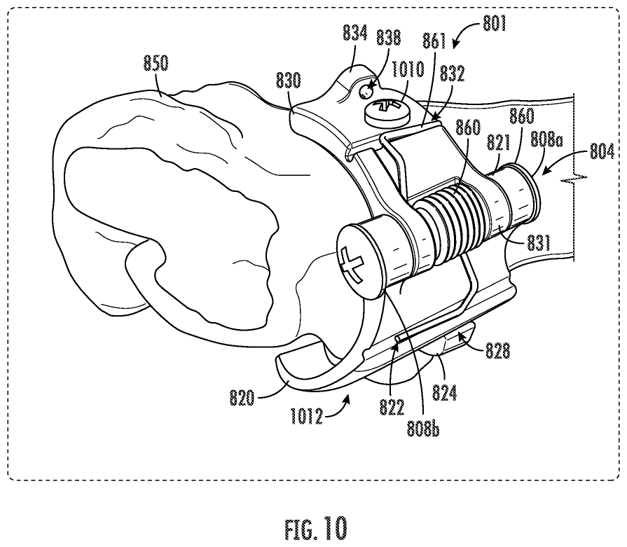

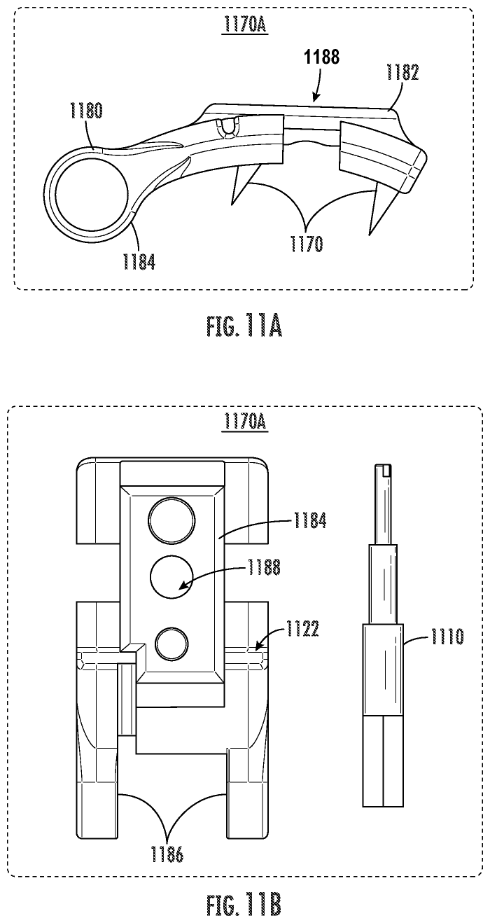

FIGS. 11A and 11B show perspective views of an example embodiment of an arm of a spring-actuated mounting apparatus in accordance with features of the present disclosure. As shown in FIG. 11A, arm 1170A may include engagement end 1182 and connecting end 1180 having prongs 1186. Arm 1170A may include a plurality of protrusions 1170 and a device holder 1184. Referring to FIG. 11B, device holder 1184 may include one or more mounting points (such as device cavity or receiver 1188). In various embodiments, a medical aid device 1110 (for example, tracking array) may be arranged within and/or affixed to arm 1170A (or another portion of spring-actuated mounting apparatus 801) through a mounting point. In some embodiments, spring-actuated mounting apparatus 801 may include a plurality of device holders 1184 and/or mounting points. In some embodiments, mounting points (such as cavity 1188) may have a pattern that matches a pattern of a corresponding portion of medical aid device 1110. For example, a medical aid cavity may have a hexagonal pattern that matches a hexagonal pattern of a bottom portion of telescoping tracking array medical aid device 1110. The hexagonal pattern of the base of the array and the multiple hexagonal hole pattern of the medical aid cavity, for example, embossed in the device holder 1184, may allow for variable positioning of an array. Paired with a telescopic shaft connection, such embodiments may allow for an optimized range of detection by a navigation device, such as a camera.

Alternative or additional mounting methods may be used according to some embodiments. For example, a medical aid device, such as medical aid device 1110, may be attached via a magnetic, clip-on, friction fit, locking mechanism, or other mechanical attachment. Embodiments are not limited in this context.

Mounting points 1188 and/or medical aid device 1110 may be used with other types of mounting devices, such as a magnetically-actuated mounting device and/or a mechanically-actuated mounting device according to some embodiments.

FIG. 12 shows a perspective view of an example embodiment of a release device and a spring-actuated mounting apparatus in accordance with features of the present disclosure. As shown in FIG. 12, a release device 1210 (for instance, a cobra retractor) may have a handle end 1211 and an engagement end 1212. In various embodiments, engagement end 1212 may be configured to engage one or more of arms 820 and 830, for example, via release attachments 824 and/or 834 and/or cavities 828 and 838, respectively, thereof.

Release device 1210 may be used to pry, force, or otherwise move one or more of arms 820 and/or 830 in a releasing direction away from each other to release spring-actuated mounting apparatus 801 (or hold spring-actuated mounting apparatus 801 in an open state). For example, as depicted in FIG. 12, engagement end 1212 may engage arm 820 and 830, attaching to arm 830 such that pulling down on handle end 1211 may pry arm 830 away from arm 820. Although a particular example of a release device and release method is depicted in FIG. 12, embodiments are not so limited, as other release devices, holding devices, and/or release methods capable of operating according to some embodiments are contemplated herein. For example, a Gelpi retractor may be used as a release device according to some embodiments.

FIG. 13 shows a side view of a block diagram of an example of an embodiment of a linkage-tensioning mounting apparatus in accordance with features of the present disclosure. As shown in FIG. 13, a mounting apparatus 1301 may include a pair of opposing arms 1320 and 1330 configured to be arranged around a portion of a human body 1350, such as a femur. Each of arms 1320 and 1330 may have a connection end 1322 and 1332, respectively, configured to be coupled to, arranged around, or otherwise engaged with clamping assembly 1310. Arms 1320 and 1330 may have an engagement end 1323 and 1333, respectively, to engage a portion of femur 1350 when mounting apparatus 1301 is affixed to femur 1350. In various embodiments, one or both of arms 1320 and 1330 may include protrusions 1370. In some embodiments, protrusions 1370 may include teeth, spikes, needles, bumps, and/or similar structures that may operate to engage (for example, grip, dig or bite into, and/or the like) femur 1350 and/or associated anatomical structures to further facilitate attachment of mounting apparatus 1301 to femur 1350.

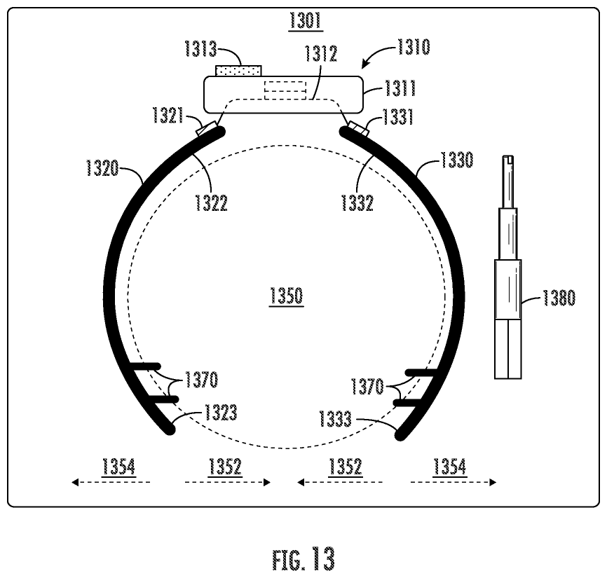

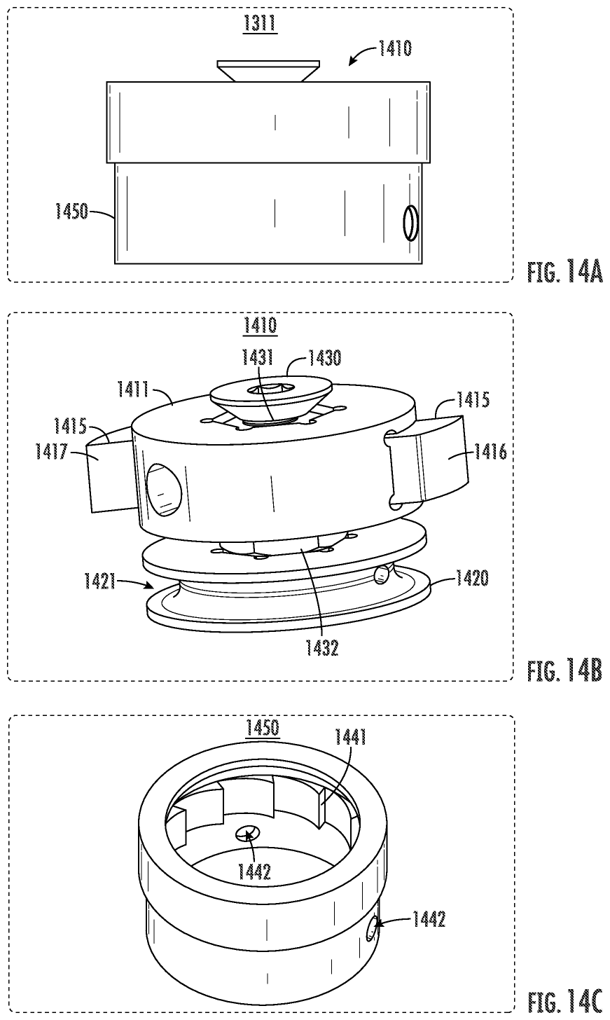

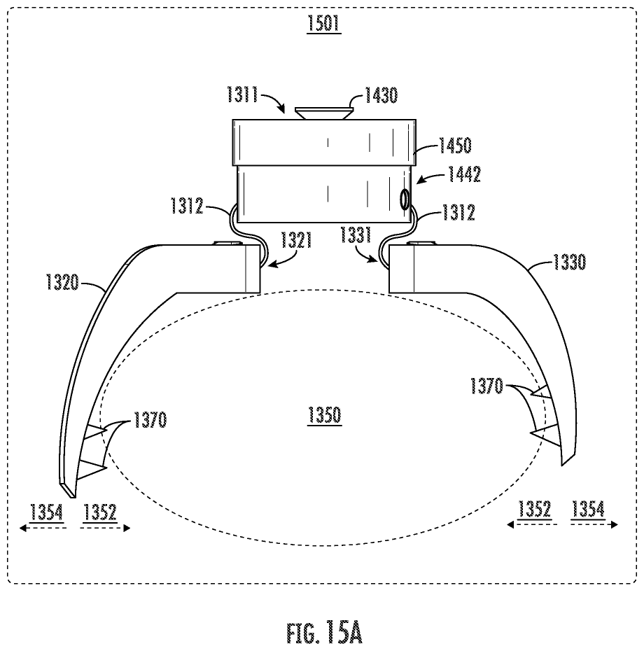

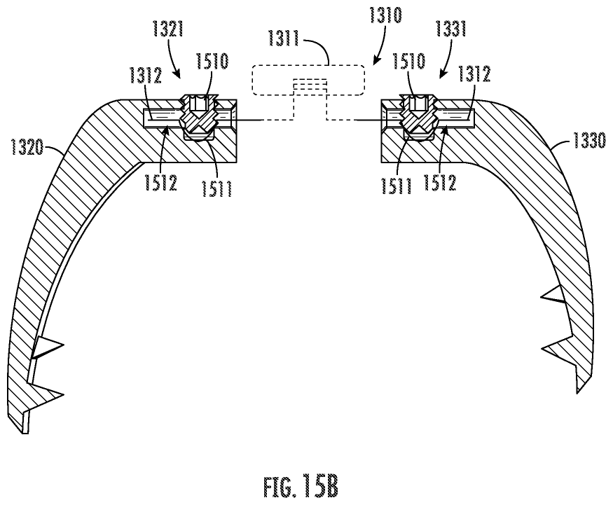

In some embodiments, the configuration (for instance, shape, size, contour, and/or the like) of opposing arms 1320 and 1330 may be the same or substantially the same. In other embodiments, the configuration of opposing arms 1320 and 1330 may be different (see, for example, FIGS. 15, 16A, 18A, and 21A-21C). For example, arm 1320 may be a medial arm configured to engage a medial or substantially medial side of femur 1350. Arm 1330 may be a lateral arm 1330 configured to engage a lateral or substantially lateral side of femur 1350.

In various embodiments, at least one of arms 1320 and 1330 may be coupled to a clamping assembly 1310. In some embodiments, clamping assembly 1310 may include a tensioning mechanism 1311 and a linkage 1312. The linkage may include a cable, such as a metal cable, including a cable composed of a stainless steel alloy that may, in some embodiments, be coated with a biocompatible polymer including nylon, and/or the like. Other options for the linkage material may include high strength fibrous materials such as Kevlar. Non-limiting examples of tensioning mechanisms 1311 may be or may include ratchet-based mechanisms used alone or in combination with a linkage-tensioning mechanism or a rack-and-pinion mechanism.

In some embodiments, both of arms 1320 and 1330 may be coupled to clamping assembly 1310, for instance, via linkage 1312. In other embodiments, only one of arms 1320 and 1330 may be coupled to clamping assembly 1310 and/or linkage 1312. For example, only a medial arm may be forced by clamping assembly 1310. In such embodiments, the arm not forced by clamping assembly 1310 (for instance, a “fixed arm”) may be coupled to clamping assembly 1310 and/or portions thereof (such as a hub or tightening mechanism) via a joint (for example, a dovetail joint) or other connection.

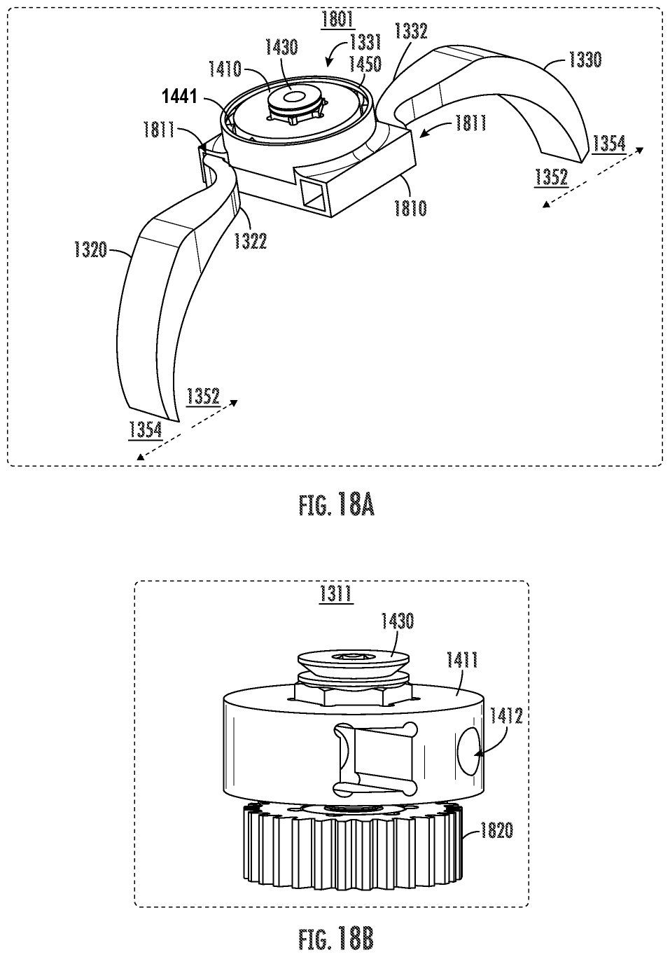

In some embodiments, arm 1320 and/or 1330 may include a connector 1321 and 1331, respectively, configured to be coupled to or otherwise engage linkage 1312 and/or clamping assembly 1310. In various embodiments, tensioning mechanism 1311 may be configured to tension or tighten linkage 1312. For example, linkage 1312 may be tightened to generate a pulling force by shortening linkage 1312, for instance, by reducing a length of linkage 1312 outside of tensioning mechanism 1311 (for example, linkage 1312 may be wound or otherwise arranged within tensioning mechanism 1311 (see, for example, FIGS. 14A-14C and 18B)). The pulling force may operate to force arm 1320 and/or 1330 in clamping direction 1352. In various embodiments, at least a portion of linkage 1312 may be stretchable, flexible, or otherwise exhibit elastic properties. In such embodiments, linkage 1312 may be tensioned by pulling on linkage 1312 to increase the elastic force of linkage 1312, thereby pulling arm 1320 and/or 1330 in clamping direction 1352. Arm 1320 and/or 1330 may be forced in clamping direction 1352 via the pulling force and/or the elastic force.

Tensioning of linkage 1312 may generate a clamping force or tension causing arms 1320 and/or 1330 to move in clamping direction 1352 toward femur 1350. The clamping force may operate to rigidly affix mounting apparatus 1301 to femur 1350. When mounting apparatus 1301 is rigidly affixed to femur 1350, mounting apparatus 1301 and components thereof (for example, arms 1320 and 1330) may have limited or no freedom of movement, either rotationally or axially.

The clamping force generated by tightening linkage 1312 may be released, reduced, or even completely eliminated. For example, tensioning mechanism 1311 may be operated to loosen or relax linkage 1312, for instance, by increasing a length of linkage 1312 outside of tensioning mechanism 1311 and/or reducing an elastic force generated via tensioning linkage 1312. Releasing the clamping force may allow arm 1320 and/or 1330 to move in a releasing direction 1354 away from femur 1350.

In some embodiments, tensioning mechanism 1311 may include or may be otherwise associated with a release mechanism 1313 configured to release tension (and therefore, the clamping force) in linkage 1312 generated by tensioning mechanism 1311. In some embodiments, release mechanism 1313 may include a button, lever, or other element that may release all (or substantially all) of the clamping force responsive to being actuated (i.e., pressing on release mechanism 1313 may instantaneously or substantially instantaneously release linkage 1312 and, therefore, arm 1320 and/or 1330). In various embodiments, release mechanism 1313 may operate to allow tensioning mechanism 1311 to move in a direction that reduces or eliminates the tension of linkage 1312. For example, rotating tensioning mechanism 1311 clockwise may increase the tension of linkage 1312. Release mechanism 1313 may operate as a catch or release to allow tensioning mechanism to rotate in a counterclockwise direction to release the tension of linkage 1312. In another example, release mechanism 1313 may include a structure on or within a portion of tensioning mechanism 1311 that allows a portion of tensioning mechanism 1311 to move to release the tension of linkage 1312 (for example, see element 1431 of FIG. 14B).

In some embodiments, a medical aid device 1380 may be attached to mounting apparatus 1301. For example, in various embodiments, mounting apparatus 1301 may include a device holder (not shown; see, for example, FIG. 11B) that may include or be used as one or more mounting points, cavities, and/or the like for coupling one or more medical aid devices 1380 to mounting apparatus 1301. In some embodiments, mounting apparatus 1301 may include a plurality of device holders and/or mounting points. In some embodiments, mounting points may have a pattern that matches a pattern of a corresponding portion of medical aid device 1380. For example, a cavity or other mounting point may have a hexagonal pattern that matches a hexagonal pattern of a telescoping tracking array medical aid device 1380. The hexagonal pattern of the base of the array and the multiple hexagonal hole pattern of the mounting point, for example, embossed in the device holder, may allow for variable positioning of an array. Paired with a telescopic shaft connection, such embodiments may allow for an optimized range of detection by a navigation device, such as a camera. In some embodiments, medical aid device 1380 may be affixed directly to a portion of mounting apparatus 1301, such as arms 1320 or 1330 and/or a portion of mounting apparatus 1310. Embodiments are not limited in this context.

Tensioning mechanism 1311 may operate according to various techniques to tension and/or relax linkage 1312. For example, tensioning mechanism 1311 may be or may include a ratcheting system, a rack-and-pinion system, and/or the like. FIGS. 14A-14C show a ratchet-based tensioning mechanism of a linkage-tensioning mounting apparatus in accordance with features of the present disclosure. As shown in FIG. 14A, tensioning mechanism 1311 may include a ratcheting system having a ratchet device 1410 arranged within a housing 1450 In some embodiments, tensioning mechanism 1311 ratcheting system may operate to tension cable via a ratcheting mechanism or process. FIG. 14B depicts a perspective view of ratchet device 1410 according to some embodiments, and FIG. 14C shows a perspective view of housing 1450 according to various embodiments.

Referring to FIGS. 14B and 14C, ratchet device 1410 may include a shaft 1430 and a fastener 1432 that may operate as a drive shaft for ratchet device 1410. In some embodiments, shaft 1430 may be a screw threaded to correspond to internal threads of a hex nut fastener 1432. Linkage 1312 may be passed through spool 1420 and/or wound within groove 1421 of spool 1420. Ratchet device 1410 may include a body or disk 1411 having one or more teeth 1415. In some embodiments, teeth 1415 may have a first side 1416 and a second side 1417. A force on first side 1416 may cause teeth 1415 to compress or retract within body 1411, while a force on second side 1417 may not cause teeth 1415 to compress or move within body 1411. In various embodiments, teeth 1415 may be biased toward the outside of body 1411, for example, by a spring or other mechanism within body 1411. In various embodiments, body 1411 and spool 1420 may be coupled (for example, via the drive shaft formed by shaft 1430 and/or fastener 1432) such that rotation of body 1411 may cause a corresponding rotation in spool 1420.

Housing 1450 may have internal teeth 1441 that correspond with teeth 1415. Rotation of ratchet device 1410 within housing 1450 in a first or tensioning direction (for instance, clockwise) may cause teeth 1441 to press on first side 1416 of teeth 1415 such that teeth 1415 are compressed or retracted into body 1411, allowing rotation of ratchet device 1410 in the tensioning direction. In some embodiments, ratchet device 1410 may be coupled to linkage 1312, for instance, via a coupling with spool 1420, such that rotation of ratchet device 1410 within housing 1450 in the tensioning direction may cause tensioning of linkage 1312 (for instance, via a corresponding rotation of spool 1420). In various embodiments, linkage 1312 may protrude from housing via one or more openings 1442 to allow linkage 1312 to be connected to arm 1320 and/or 1330.

Teeth 1441 may engage side 1417 of teeth 1415 to prevent rotation of ratchet device 1410 in a second or relaxing direction (for instance, counterclockwise or in a direction opposite the tensioning direction). Accordingly, release mechanism 1313 may be included in or operably coupled to a portion of tensioning mechanism 1311 to allow ratchet device 1410 to rotate in the releasing direction. For example, release mechanism 1313 may cause teeth 1415 to retract into body 1411 such that teeth 1441 do not engage side 1417 when ratchet device 1410 is rotated in the releasing direction. In another example, ratchet device 1410 may be moved upward within housing 1450 to disengage teeth 1441 from teeth 1415. For instance, shaft 1430 may have release mechanism 1431 in the form of a ridge or undercut that may be used to pry, pull, or otherwise force ratchet device 1410 to move upward (or downward) within housing 1450 to allow body 1411 to rotate in the releasing direction such that teeth 1415 do not engage teeth 1441.

Accordingly, in one embodiment, tensioning device 1311 may include a screw 1430 and hex nut 1432 that act in combination as a driveshaft, a ratcheting disk 1411 with teeth 1415 that compress linearly as disk 1411 is turned in a tensioning direction inside housing 1450 with internal tooth pattern 1441, spool 1420 that retains cable 1312, and a retaining screw 1430 (or another fastener that is not shown in FIGS. 14A-14C) that fixates spool 1420 to the bottom of housing 1450. Tooth pattern 1441 may prevent rotation of ratcheting disk 1411 in the relaxing direction by engaging side 1417 of teeth. The driveshaft mates to the top of spool 1420 and couples spool 1420 with ratcheting disk 1411. Cable 1312 may be passed through spool 1420 to collect along the spool core (for instance, groove 1421) as the driveshaft is turned using a hex drive (or other type of drive) slot on the head of screw 1430. Ratcheting disk 1411 may only allow rotation in one direction while engaged with spool 1420 (i.e., the tensioning direction). Undercut 1431 on screw 1430 may allow for ratcheting disk 1411 to be easily pulled upward to disengage from spool 1420, thereby allowing cable 1312 to unwind, thereby releasing the clamping force.

Although FIGS. 14A-14C depict a particular embodiment of a ratcheting system, embodiments are not so limited. For example, different types of ratcheting systems or tensioning systems may be used in accordance with various embodiments, such as gear-and-pawl systems (for instance, a ratchet or gear with a pawl to prevent unwanted motion), rack-and-pinion systems, derivatives thereof, combinations thereof, other configurations of ratchet systems, and/or the like.

FIG. 15A shows a side view of a first embodiment of a free-arm linkage-tensioning mounting apparatus in accordance with features of the present disclosure. In some embodiments, a mounting apparatus 1501 may have a free-floating or free-arm configuration that includes two separate arms 1320 and 1330 that are not directly connected to each other (besides being connected via linkage 1312). In some embodiments of mounting apparatus 1501, arm 1320 may be a medial arm and arm 1330 may be a lateral arm. In some free-arm embodiments, tensioning mechanism 1311 may be coupled to a support or base (see, for example, FIGS. 16A and 16B) that may facilitate positioning of arm 1320 and/or 1330 and/or alignment of linkage 1312.

In some embodiments, linkage 1312 may be coupled to arm 1320 and/or 1330 using various techniques. For example, connector 1321 or 1331 may include a set-screw mechanism, a linkage seat, and/or the like. FIG. 15B shows an internal side view of a linkage-tensioning mounting apparatus with a set-screw connector in accordance with features of the present disclosure. In some embodiments, connector 1321 or 1331 may operate to couple linkage 1312 to arm 1320 and/or 1330 via a set screw 1510. Linkage 1312 may be arranged within a linkage cavity 1511. Set screw 1510 may be threaded into a threaded cavity 1512, thereby intersecting linkage 1312 and holding linkage 1312 in place within arm 1320 and/or 1330. Although mounting apparatus 1501 is depicted with set screw connectors 1321 and 1331, embodiments are not so limited, as any type of connector capable of operating according to some embodiments may be used in combination with mounting apparatus 1501 (for instance, a linkage-seat connector).

FIG. 16A shows a perspective view of a second embodiment of a free-arm linkage-tensioning mounting apparatus in accordance with features of the present disclosure. FIG. 16B shows a perspective view of a tensioning mechanism for the free-arm linkage-tensioning mounting apparatus of FIG. 16A. As shown in FIGS. 16A and 16B, tensioning mechanism 1311 may be arranged in or otherwise coupled to a base 1610. In various embodiments, base 1610 may have openings 1611 for linkage 1312 to pass through base 1610 from tensioning mechanism 1311 and connect via connector 1321 and/or 1331 to arm 1320 and/or 1330, respectively. In some embodiments, base 1610 may be configured to align linkage 1312, for example, when mounting apparatus 1601 is tensioned in a clamping position.

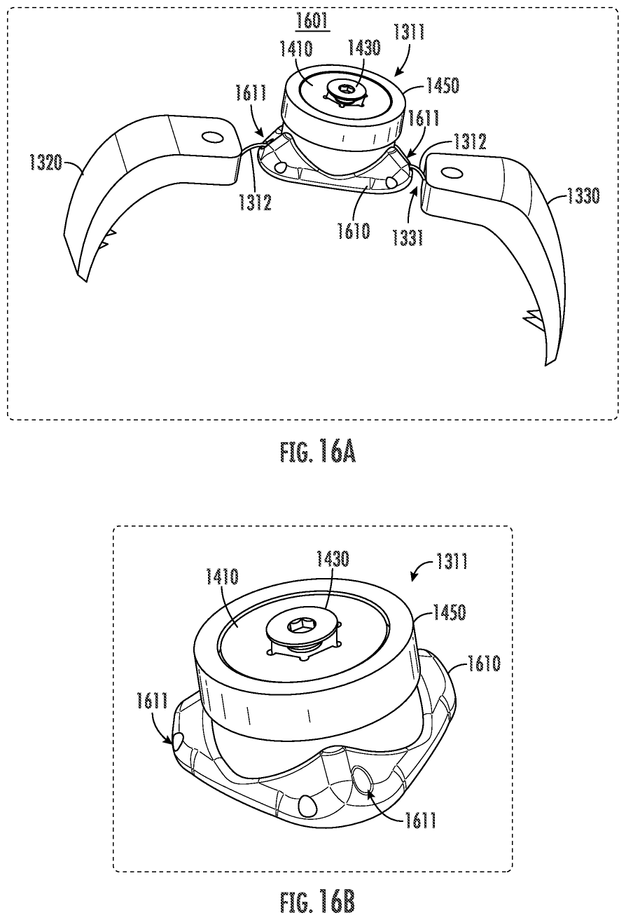

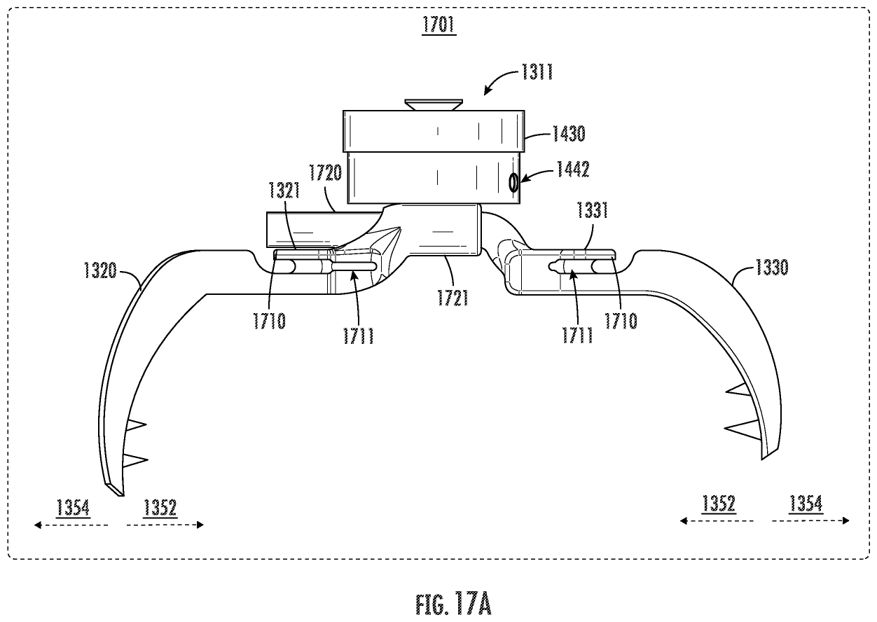

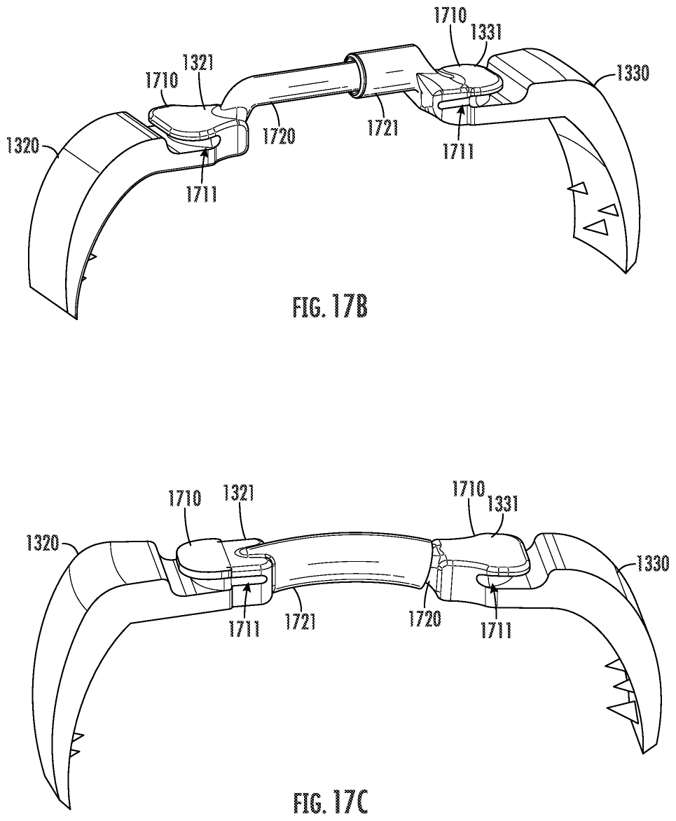

FIG. 17A shows a side view of a fixed-arm or track-arm linkage-tensioning mounting apparatus in accordance with features of the present disclosure. As shown in FIG. 17A, arms 1320 and 1330 may be coupled via a straight-track configuration that may include a post or shaft 1720 and a corresponding cylinder 1721 configured to receive post 1720. Although post 1720 is depicted as being associated with arm 1330 and cylinder 1721 with arm 1320, embodiments are not so limited, as post 1720 may be associated with arm 1320 and cylinder 1721 with arm 1330. In some embodiments, the track (i.e., post 1720 and cylinder 1721) may operate to align arms 1320 and 1330, for example, along a central axis while allowing rotation (for instance, into and/or out of the page of FIG. 17A).

Tensioning mechanism 1311 may be coupled to one of arms 1320 or 1330, for example, by being affixed to cylinder 1721. In some embodiments, arm 1320 or 1330 may have a linkage-seat connector 1321 or 1331, respectively, having a tab or ridge 1710 and a groove 1711. In various embodiments, linkage 1312 may be wound around groove 1711 with ridge 1710 preventing upper movement of linkage 1312 as linkage 1312 is tensioned. For example, linkage 1312 may include a cable having two loops, with one loop at each end fixed around grooves 1711. Accordingly, as linkage 1312 is tensioned via tensioning mechanism 1311, arm 1330 may be moved in clamping direction 1352 toward arm 1320.

FIG. 17B shows a perspective view of track-arms for the linkage-tensioning mounting apparatus of FIG. 17A according to a first embodiment. FIG. 17C shows a perspective view of a second embodiment of track-arms for a track-arm linkage-tensioning mounting apparatus. As shown in FIG. 17C, arms 1320 and 1330 may be connected via curved-track configuration having a curved post 1720 and corresponding cylinder 1721. In both the straight-track and curved track configurations, cylinder 1721 allows post 1720 to remain aligned as linkage 1312 is being tensioned to cause arm 1320 and/or 1330 to move in clamping direction. For example, the track (i.e., post 1720 and cylinder 1721) may operate to align arms 1320 and 1330, for example, along a central axis while allowing rotation (for instance, into and/or out of the page of FIG. 17A-17C about the longitudinal axis of post 1720 or cylinder 1721).

Although mounting apparatus 1701 is depicted with linkage-seat connectors 1321 and 1331, embodiments are not so limited, as any type of connector capable of operating according to some embodiments may be used in combination with mounting apparatus 1701 (for instance, a set-screw connector).

FIG. 18A shows a perspective view of a rack-and-pinion mounting apparatus in accordance with features of the present disclosure. FIG. 18B shows a perspective view of a tensioning mechanism for the rack-and-pinion mounting apparatus of FIG. 18A. As shown in FIGS. 18A and 18B, tensioning mechanism 1311 may include a ratchet 1410 having teeth 1415 arranged within a housing 1450 with internal teeth 1441 arranged on an internal sidewall of housing 1450. Ratchet 1410 and housing 1450 may operate the same or substantially similar as described with respect to FIGS. 14A-3C, except that instead of rotation of ratchet 1410 causing rotation of a spool, rotation of ratchet 1410 may cause a corresponding rotation of gear or pinion 1820. In other embodiments, different types of ratcheting systems or tensioning systems may be used in accordance with various embodiments, such as gear-and-pawl systems, rack-and-pinion systems, other configurations of ratchet systems, and/or the like.

Arms 1320 and 1330 may have connection ends 1322 and 1332, respectively, inserted within openings 1811 of base 1810. In some embodiments, at least one of connection ends 1322 and 1332 may have teeth (not shown) corresponding to gear 1820, for example, to operate as a rack to pinion 1820. Rotation of body 1411 may cause a corresponding rotation in pinion 1820. In various embodiments, rotation of body 1411 in a first or tensioning direction (for example, clockwise) may cause pinion 1820 to pull at least one of arm 1320 and/or 1330 in clamping direction 1352. Pulling or prying body 1411 upwards disengages nut 1432 from the spool 1420 by allowing vertical clearance between the components. Pinion 1820 or Spool 1420 may rotate freely allowing the arm 1320 to move in the relaxing direction (opposite the tensioning direction). In some embodiments, only one of arms 1320 and 1330 may have a rack portion with teeth that engage pinion 1820, limiting its movement according to the rotation of the gear. As described with respect to FIGS. 14A-3C, tensioning mechanism 1311 of mounting apparatus 1801 may have a release mechanism, such as a release button or undercut of shaft 1430.

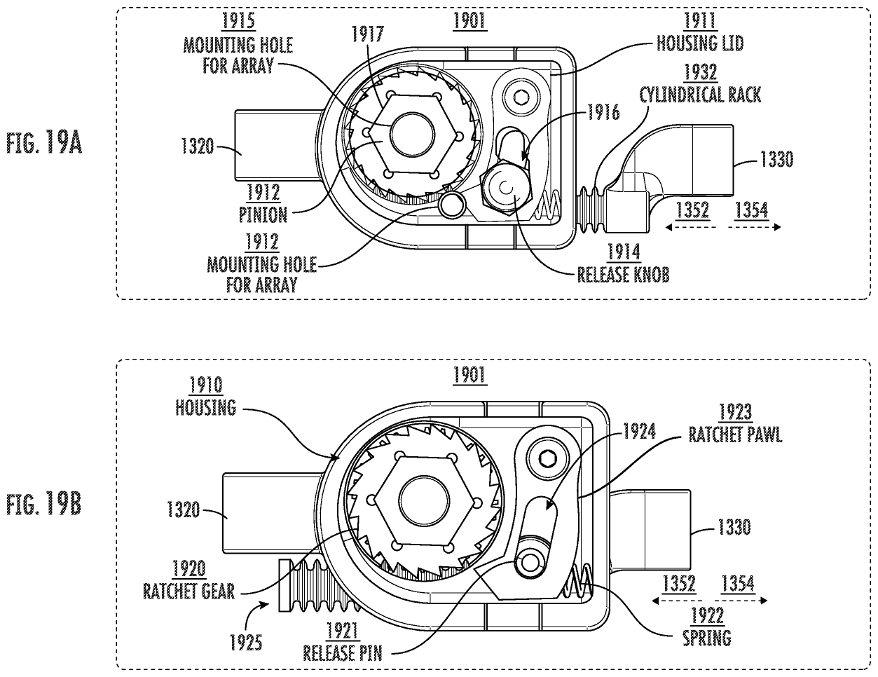

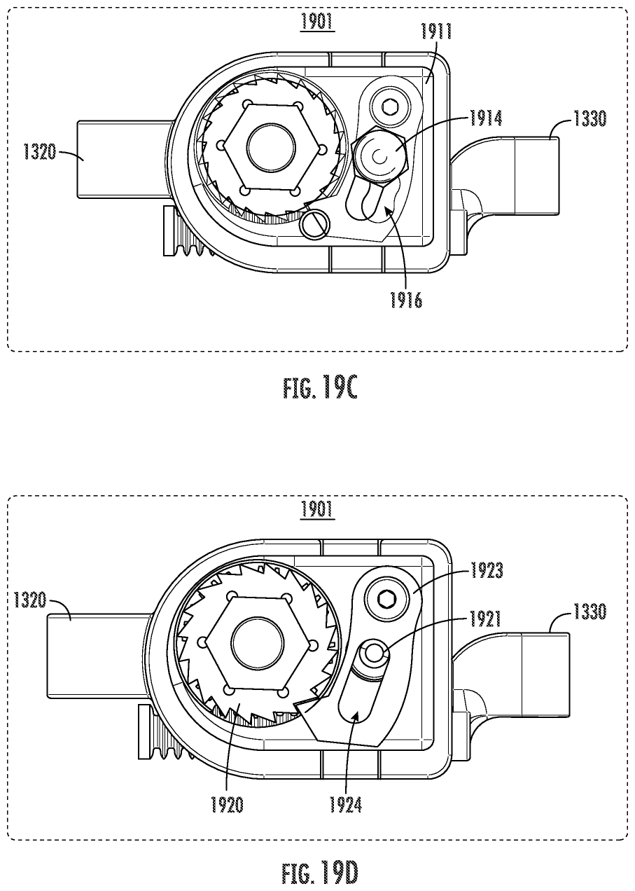

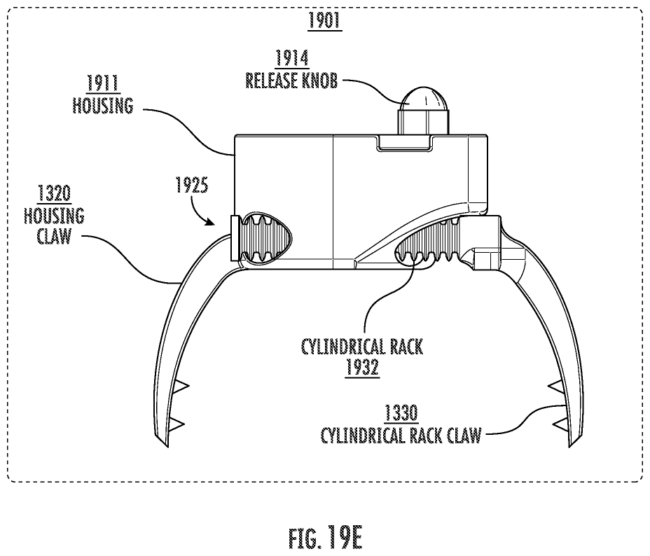

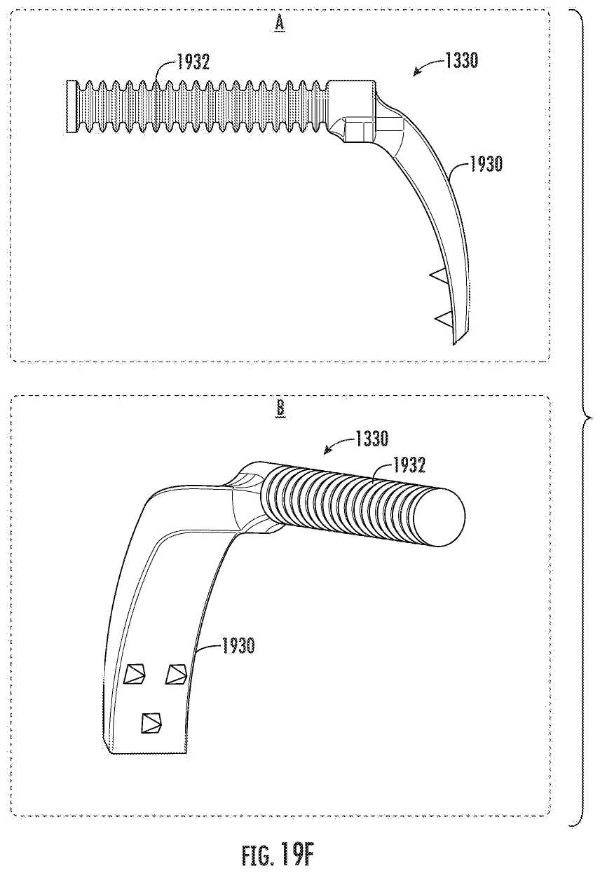

FIGS. 19A and 19B show top-down views of a second embodiment of a rack-and-pinion mounting apparatus in an open configuration in accordance with features of the present disclosure. As shown in FIGS. 19A and 19B, a rack-and-pinion mounting apparatus 1901 may include arms 1320 and 1330 arranged about a housing 1910, which may include a lid 1911. FIG. 19A depicts rack-and-pinion mounting apparatus 1901 with lid 1911 and release knob 1914, while FIG. 19B depicts rack-and-pinion mounting apparatus 1901 without lid 1911 and release knob 1914 (for example, to more clearly show the elements underneath lid 1911 and release knob 1914).

In some embodiments, arm 1320 may be an integral part of housing 1910 (see, for example, FIG. 19E) and arm 1330 may be configured to move in one of a clamping direction 1352 or a releasing direction 1354. In various embodiments, arm 1330 may have a cylindrical rack 1932 configured to engage a ratchet gear 1920.

In some embodiments, ratchet gear 1920 may be coupled to pinion gear 1912, for example, by an external hex 1917. Ratchet pawl 1923 may be configured to prevent anti-rotation of ratchet gear 1920 (or pinion gear 1912), which prevents the mechanism from loosening or backing out once mounting apparatus 1901 is attached to a bone.

In some embodiments, arm 1330 may have a cylindrical rack 1932 in place of a straight or square rack. The shape of cylindrical rack 1932 may allow for free rotation about a central axis of cylindrical rack 1932, for example, while mounting apparatus 1901 is tightening. This variability allows, among other things, for better placement in differing anatomies. Pinion gear 1912 may be contoured to the shape of the revolved teeth on the cylindrical rack 1932.

A slot 1925 in housing 1911 may be configured to align cylindrical rack 1932 and allow it to engage with pinion gear 1912. When pinion gear 1912 is turned clockwise, cylindrical rack 1932 may be pulled in clamping direction 1352 toward arm or claw 1320 on housing 1910. A ratchet pawl 1923 may be biased toward ratchet gear 1920 by a biasing element (e.g., compression spring) 1922, preventing counterclockwise rotation of gears 1912, 1920. Release element or pin 1921 may be coupled to knob 1914 (for instance, a hex knob) on the end and may be contained within slots 1916 and 1924 in ratchet pawl 1923 and housing lid 1911, respectively. The movement of release pin 1921 may be constrained by slot 1916 in housing lid 1911. In order to open mounting apparatus 1901, release pin 1921 may be moved by hand, an instrument, an automated device, and/or the like. One or more mounting holes, cavities, or other elements 1915 may be arranged on or in rack-and-pinion mounting apparatus 1901.

FIGS. 19C and 19D show top-down views of the mounting apparatus of FIG. 19A in a closed configuration in accordance with features of the present disclosure. When release pin 1921 is in the original closed position depicted in FIGS. 19C (depicted with lid 1911 and knob 1914) and 19D (depicted without lid 1911 and knob 1914 for clarity purposes), ratchet pawl 1923 is engaged with ratchet gear 1920 by compression spring 1922. When release pin 1921 is moved to the open position depicted in FIGS. 19A and 19B, release pin 1921 pushes against ratchet pawl 1923 disengaging it from ratchet gear 1920. When ratchet pawl 1923 is disengaged by release pin 1921, pinion gear 1912 may be turned counterclockwise to loosen mounting apparatus (i.e., to push arm 1330 on cylindrical rack 1932 in releasing direction 1354 away from arm 1320 on housing 1910).

FIG. 19E shows a side view of the rack-and-pinion mounting apparatus of FIG. 19A. FIG. 19F shows a side view and a perspective view of a cylindrical rack arm of the rack-and-pinion mounting apparatus of FIG. 19A.

FIG. 20A shows a perspective view and a cross-sectional view of a third embodiment of a rack-and-pinion mounting apparatus in accordance with features of the present disclosure. In particular, area A depicts a perspective view and area B depicts a transverse cross-sectional view of rack-and-pinion mounting apparatus 2001. FIG. 20B shows a pinion gear of the rack-and-pinion mounting apparatus of FIG. 20A.

As shown in FIG. 20A, a rack-and-pinion mounting apparatus 2001 may include a pinion gear 2020 arranged within a housing 2010. Pinion gear 2020 may include kick teeth 2021 configured to engage corresponding (anti-rotation) teeth on an anti-rotation hub 2022. In various embodiments, arm 1320 may be an integral part of housing 2010, while arm 1330 may have a straight rack configured to engage pinion gear 2020.

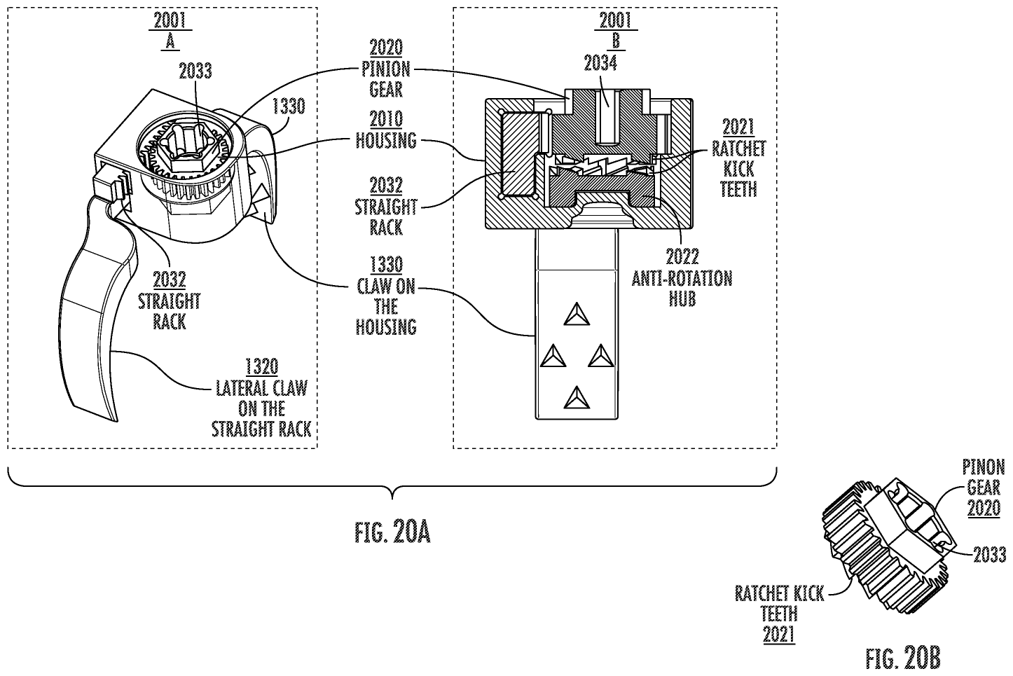

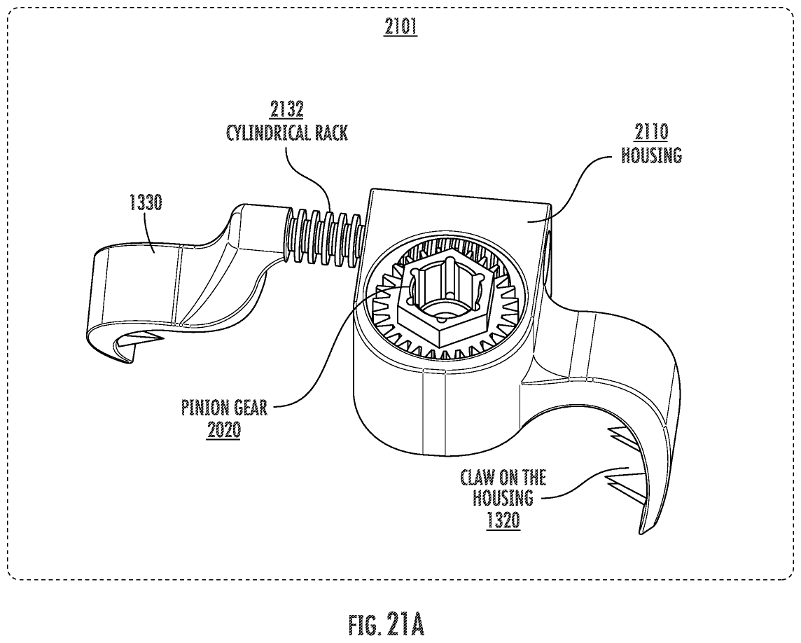

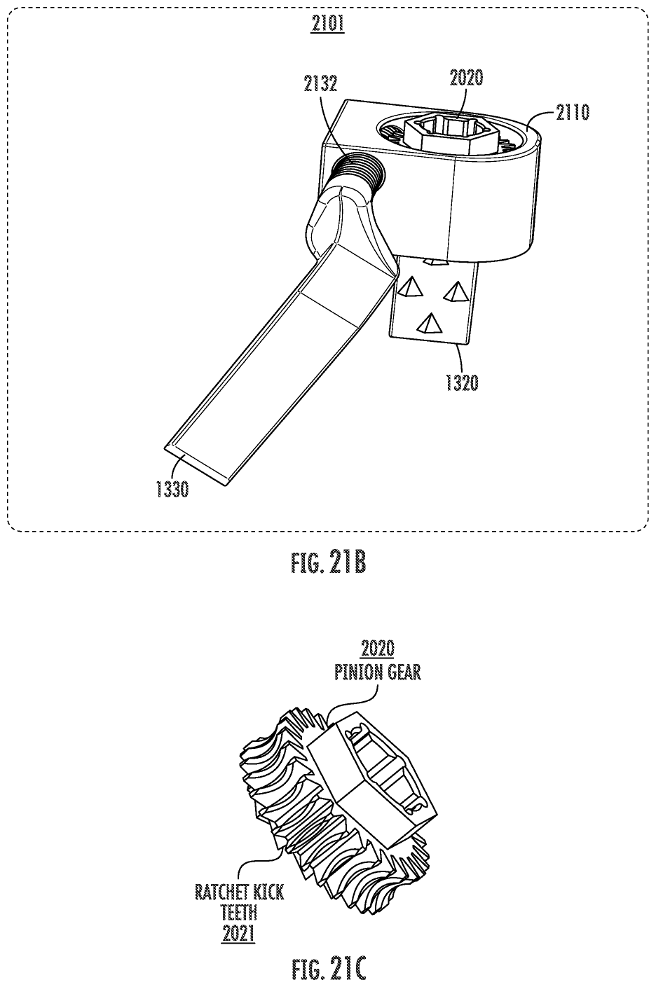

Anti-rotation hub 2022 may be in a closed position to actuate vertically during the tightening of mounting apparatus 2001. For example, a spring or other biasing element (not shown) may be configured to push anti-rotation hub toward pinion gear 2020. A smaller hex or other shaped slot 2033 contained in the center of gear 2020 may be used to release the mechanism by forcing hub 2022 to move in a downward direction or other releasing direction into an open position so that ratchet kick teeth 2021 may separate from hub 2022. Rack-and-pinion mounting apparatus 2001 may use a straight rack 2032 attached to arm 1330; however, arm 1330 may include a cylindrical rack or other type of rack according to some embodiments (see, for example, FIG. 21A). As pinion gear 2020 is rotated in a tightening direction (for example, clockwise), arm 1330 is drawn toward arm 1320 to tighten mounting apparatus 2010. Conversely, if pinion gear 2020 is rotated in a releasing direction (for example, counterclockwise), arm 1330 may be moved away from arm 1320, thereby releasing mounting apparatus 2010.

In one embodiment, after progression of one gear tooth of pinion gear 2020 on rack 2032, ratchet kicks on the pinion 2020 and hexagonal plate mesh preventing motion in the relaxing or opposite direction. A socketed screwdriver (or other driver) turns the pinion gear 2020 using hexagonal pattern (or other type of pattern to match driver) 2033 on the top of pinion gear 2020. The mechanism may be released using a hexagonal shaped rod that fits through a center 934 of hexagonal pattern 2033 atop pinion gear 2020. This tool pushes the spring loaded hexagonal plate in the downward direction separating ratchet kicks 2021, 2022. This separation allows pinion gear 2020 to rotate in the releasing direction increasing the length of opposing arms 1320 and 1330 relative to the static housing 2010.

FIG. 21A shows a perspective view of a fourth embodiment of a rack-and-pinion mounting apparatus in accordance with features of the present disclosure. FIG. 21B shows a side view of the rack-and-pinion mounting apparatus of FIG. 21A. FIG. 21C shows a pinion gear of the rack-and-pinion mounting apparatus of FIG. 21A.

As shown in FIGS. 21A and 21B, a rack-and-pinion mounting apparatus 2101 may include opposing arms 1320 and 1330 arranged about a housing. Arm 1330 may have a cylindrical rack 2132 configured to engage a pinion gear 2020. In some embodiments, pinion gear 2020 may be configured to engage an anti-rotation hub (not shown) arranged within housing 2110.

In the embodiments depicted in FIGS. 21A and 21B, arm 1330 may include a cylindrical rack. As shown in FIG. 21B, the shape of cylindrical rack 2132 may allow for free rotation about a central axis while the mechanism is tightening. This variability allows for better placement in differing anatomies. Referring to FIG. 21C, pinion gear 2120 may have contoured gears to correspond with the revolved teeth on rack 2110.

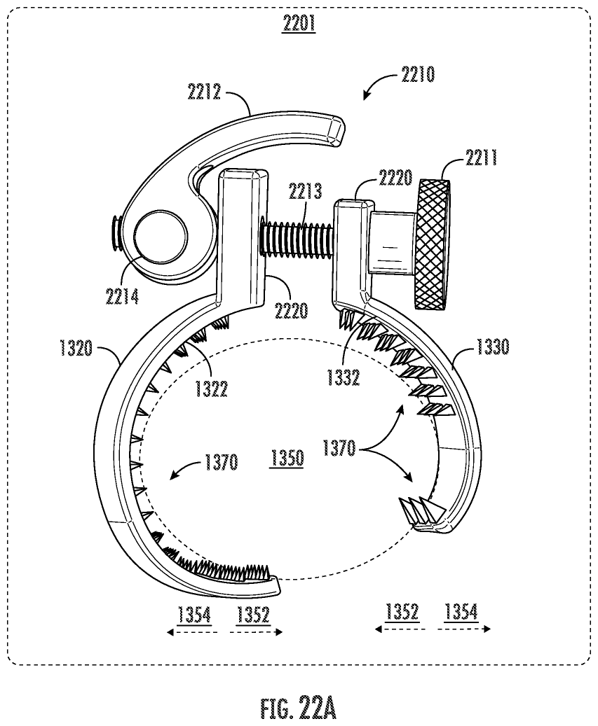

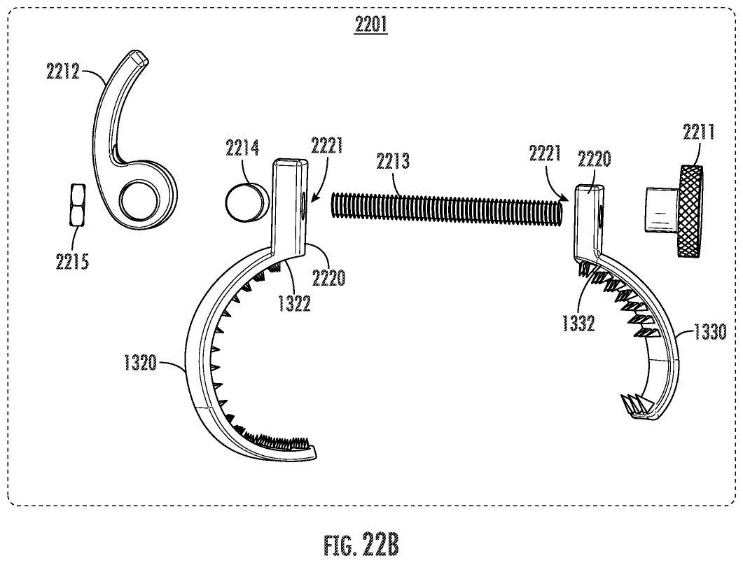

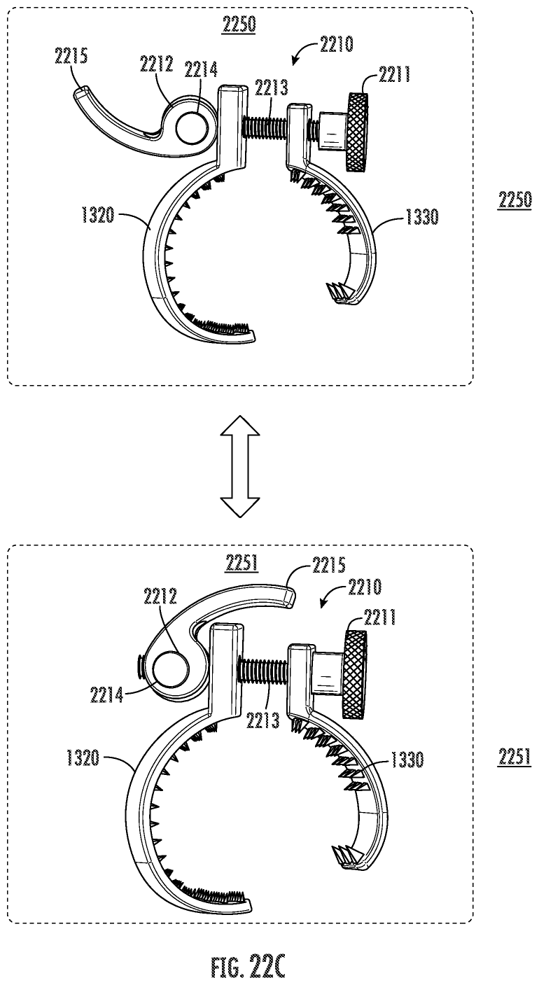

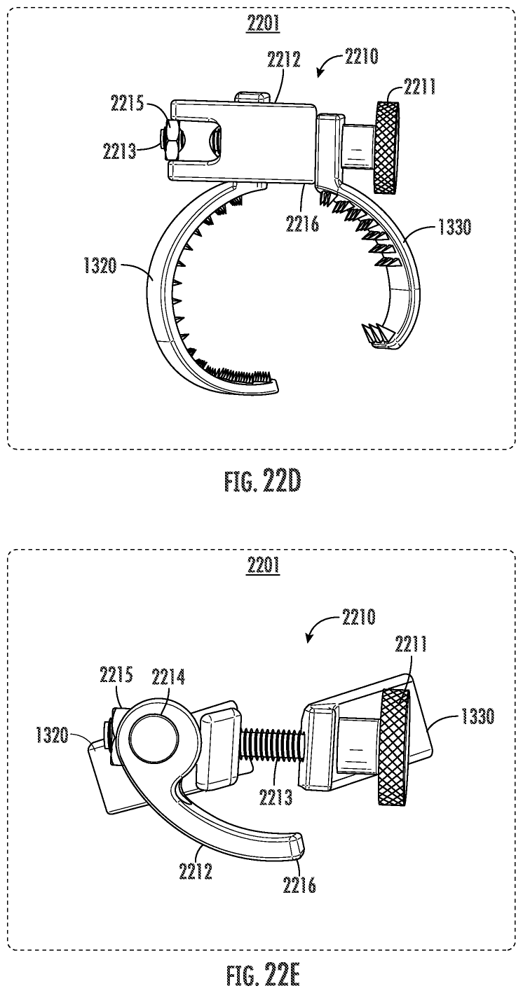

FIG. 22A shows a side view of a first embodiment of a lever-locking mounting apparatus in accordance with features of the present disclosure. FIG. 22B shows an exploded side view of the lever-locking mounting apparatus of FIG. 22A. As shown in FIGS. 22A and 22B, a lever-locking mounting apparatus 2201 may include a pair of opposing arms 1320 and 1330 coupled to a clamping assembly 2210. In some embodiments, clamping assembly 2210 may include a locking mechanism 2212 and a tensioner 2211 coupled via a connector 2213 extending through a connection end 1322 and 1332 of each of arm 1320 and 1330, respectively.

In some embodiments, arm 1320 may be a medial arm and arm 1330 may be a lateral arm. Although tensioner 2211 is depicted as being adjacent to arm 1330 and locking mechanism 2212 being adjacent to arm 1320, embodiments are not so limited. For instance, tensioner 2211 as depicted may be arranged on connector 2213 at the same end as arm 1330 and locking mechanism 2212 may be arranged on connector 2213 at the same end as arm 1320.

In some embodiments, connector 2213 may be a threaded shaft (i.e., a bolt or headless bolt) configured to be arranged through openings 2221 of prongs 2220 extending from connection ends 1322 and 1332 of arms 1320 and 1330, respectively. Tensioner 2211 may include a fastener, such as a nut or wing nut, internally threaded to correspond with external threads of connector 2213. In some embodiments, connector 2213 may be arranged through prongs 2220, a cross dowel 2214, and/or a cam or cam lever 2212. A fastener 2215, such as a hex nut, may be arranged at an end of connector 2213 opposite tensioner 2211, for example, to hold cam 2212 in place on connector 2213. In some embodiments, fastener 2215 may be a head of a bolt connector 2213 instead of being a separate fastener.

Tensioner 2211 may be configured to move in a first or tensioning direction (for example, clockwise rotation) to force at least one of arms 1320 and/or 1330 to move in clamping direction 1352 toward femur 1350. Tensioner 2211 may be configured to move in a second or relaxing direction (for example, counterclockwise or otherwise opposite the tensioning direction) to allow the at least one of opposing arms 1320 or 1330 to move in releasing direction 1354 away from femur 1350.

FIG. 22C shows a locking/unlocking process for the lever-locking mounting apparatus of FIG. 22A. In unlocked position 2250, locking mechanism 2212 is in an unlocked or open position, for example, with a handle end 2216 moved away from tensioner 2211. In locked position 2251, locking mechanism 2212 is in a locked or closed position, for example, with handle end 2216 positioned toward tensioner 2211. In some embodiments, movement of locking mechanism 2212 into locked position 2251 may cause one or both of arms 1320 and 1330 to move closer to each other. For example, placement of locking mechanism 2212 into locked position 2251 may cause arm 1330 to move closer to arm 1320. In some embodiments, placement of locking mechanism 2212 into locking position 2251 may prevent movement of tensioner 2211 and/or arms 1320 and 1330 rotationally and/or in clamping direction 1352 and/or releasing direction 1354.

Mounting apparatus 2201 may be positioned around femur 1350 (not shown) with locking mechanism 2212 in unlocked position 2250. Tensioner 2211 may be engaged with arm 1330, for example, contacting prong 2220 of arm 1330. Movement of tensioner 2211 in the tensioning direction, for example, via rotating tensioner 2211 clockwise, may push arm 1330 in clamping direction 1352 to contact femur 1350. Tensioner 2211 may be moved until arms 1320 and 1330 are sufficiently engaged with femur 1350, for example, rigidly attached and/or otherwise a secure or “snug” fit. Locking mechanism 2212 may be moved into locked position 1151, which may lock arms 1320 and 1330 and/or bring arms 1320 and 1330 closer together to provide additional clamping force, fixation of projections 1370, and/or prevent mounting apparatus 2201 from loosening.

FIGS. 22A-22C depict mounting apparatus 2201 with locking mechanism 2212 in an upright or vertical position. In some embodiments, locking mechanism 2212 may be arranged in a horizontal or side-locking position. FIG. 22D shows a side view of the lever-locking mounting apparatus of FIG. 22A in a side-locking position. FIG. 22E shows a top view of the lever-locking mounting apparatus of FIG. 22A in a side-locking position. In the orientation depicted in FIG. 22A, a medical aid device may be affixed to a top portion of arm 1320 or 1330.

Accordingly, in some embodiments, a mounting apparatus 2201 may include a clamp operating via manual input to bring together opposing arms 1320 and 1330 around a portion of the human body with a locking/tightening mechanism consisting of a lever cam mechanism on one side (locking mechanism 2212) with a wing nut on the other side (tensioner 2211). In some embodiments, locking mechanism 2212 may operate to increase the clamping force significantly and allow for easy fixation and release. In some embodiments, arms 1320 and 1330 may feature an angular offset from one another that produces a moment about the central axis of the portion of the human body to increase clamping force.

In some embodiments, accordingly, mounting apparatus 2201 may operate via a mechanism that grips both sides of a portion of the human anatomy, such as the proximal end of femur 1350 distal to the femoral neck cut. A clamping force may be transferred via a bolt (connector 2213) between the medial and lateral arms (arms 1320 and 1330, respectively) that may be initially hand tightened (for example, via tensioner 2211) and then fixated in rigid attachment through the levering action of a cam or lever (locking mechanism 2212).

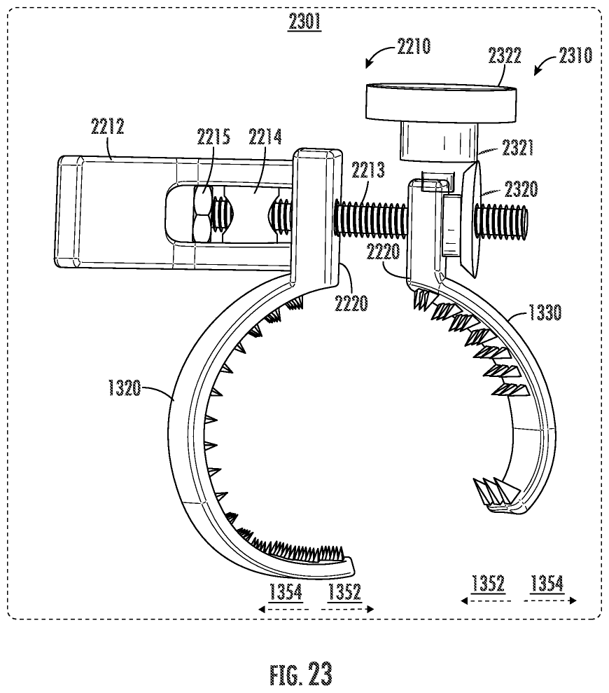

FIG. 23 shows a side view of a bevel-gear embodiment of a lever-locking mounting apparatus in accordance with features of the present disclosure. As shown in FIG. 23, a mounting apparatus 2301 may include a bevel-gear tensioner 2310 having an internally-threaded bevel gear 2320 engaged with an input bevel gear 2321. In some embodiments, input bevel gear 2321 may have or may be coupled to a head 2322 configured to engage a tool that may rotate input bevel gear 2321. For example, a drive, recess, shape, protrusion, or other element (not shown) may be arranged on top of head 2322 to receive a tool (for instance, a screwdriver, a hex driver, and/or the like) for rotating input bevel gear 2321.

Rotation of input bevel gear 2321 may cause a corresponding rotation in internally-threaded bevel gear 2320. In some embodiments, internally-threaded bevel gear 2320 may operate the same or substantially similar to tensioner 2211 of FIGS. 22A-22E except that threaded bevel gear 2320 may be rotated via rotation of input bevel gear 2321 instead of through direct rotation of tensioner 2211. In this manner, arm 1320 and/or arm 1330 may be tensioned and, therefore, moved in clamping direction 1352 or releasing direction 1354. from a position above mounting apparatus 2301. In the example depicted in FIG. 23, lever mechanism 2212 may be configured in the horizontal position.

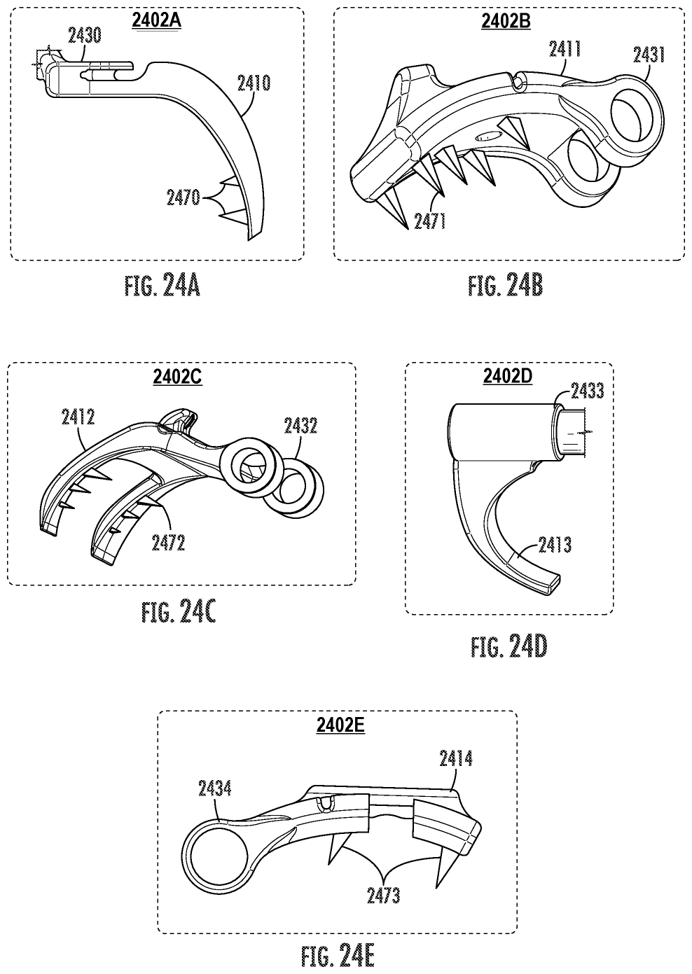

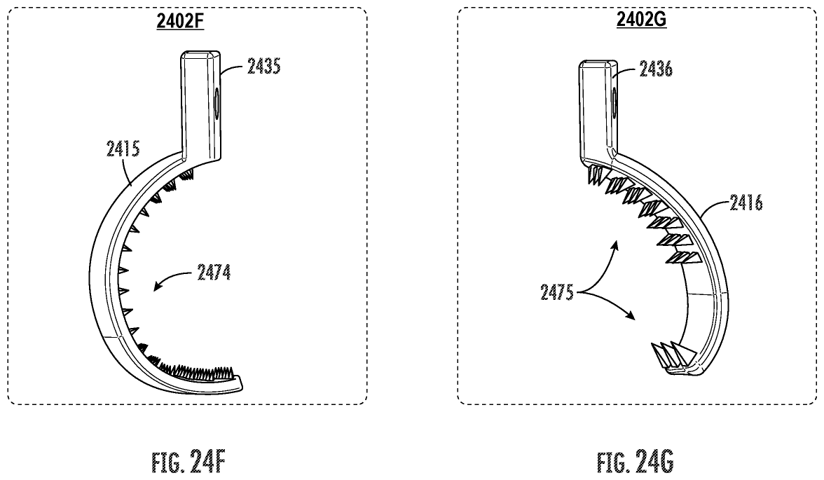

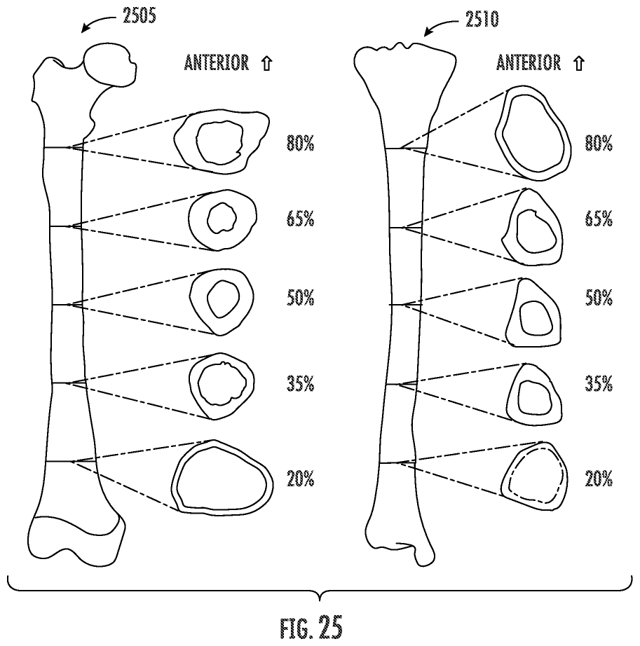

FIGS. 24A-24G depict perspective views of example embodiments of arms of a mounting apparatus in accordance with features of the present disclosure. In some embodiments, mounting structures may include one or more arms with various structures, such as mounting elements, protrusions, and/or the like. FIGS. 24A-24G depict arms 2402A-2402G, respectively, with various different types of jaws 2410-2416. In some embodiments, jaws 2410-2416 may differ with respect to multiple characteristics, such as materials, curvature, length, thickness, and/or the like. In various embodiments, arms 2402A-2402G may include different types of mounting structures or sections 2430-2436 configured to mount to different types of clamping assemblies and/or portions thereof. In exemplary embodiments, arms 2402A-2402G may include different types of protrusions 2470-2475. In various embodiments, protrusions 2470-2475 may include claw or claw-like structures configured to assist arms 2402A-2402G in gripping a portion of human anatomy, such as a femur or other bony anatomical structure. For example, protrusions 2470-2475 may include structures configured to dig into a bony anatomical structure to facilitate arms 2402A-2402G mounting to the bony anatomical structure and/or portions thereof (for example, greater or lesser trochanter). Embodiments are not limited in this context.

In some embodiments, the elements of arms 2402A-2402G may be interchangeable and/or used in combination, including with arms 202a, 202b, 602, 610, (for example, one or more of arms may be an offset arm and/or a ball-and-socket arm), 820, 830, 1320, and/or 1330. For example, arm 202a may include one or more of protrusions 2470 or 2475. In another example, arm 820 may have a curvature the same or substantially the same as arm 2402F, with protrusions 2471. In general, in some embodiments, the dimensions, spacing (for instance, distance between arms 202a and 202b), and other configurations of a mounting apparatus and/or portions thereof may be in a range suitable for affixing the mounting apparatus to a corresponding portion of the human body, such as around a femur or other bone structure. Embodiments are not limited in this context.

A mounting apparatus, clamping assembly, medical aid device, and/or components thereof may be made from various materials. Non-limiting example materials may include titanium, cobalt chrome, stainless steel, ceramic, polymers, variations thereof, alloys thereof (if applicable), combinations thereof, coatings thereof (for example, each of the aforementioned materials may be included as a coating for any other material), and/or other biocompatible materials. In some embodiments, the exterior surface of any component of a magnetically-actuated mounting apparatus may be porous and/or semi-porous. Various manufacturing techniques may be used to manufacture components of a mounting apparatus. For example, components of a mounting apparatus may be cast, additively manufactured, molded, machined, printed (for instance, via three-dimensional (3D) printing techniques), combinations thereof, and/or the like.

In some embodiments, at least a portion of a mounting apparatus may be formed of flexible material, allowing at least some measure of bending, twisting, flexing, or other movement of components. For example, arms 202a, 202b, 602, 820, 830, 1320, and/or 1330 may be formed of an at least partially flexible material. Flexible members may allow for transfer of the clamping force of a clamping assembly in the active state to the bony structure inclusive of adaptable tissue contacting pads that augment clamp stability during navigated surgery.

FIG. 25 shows cross sections of bone anatomical structures, for example, a femur 2505 and a tibia 2510. As indicated by the cross sections along the diaphysis for femur 2505 and tibia 2510, bone anatomy may have different dimensions (for instance, diameters) and shapes. In addition, bone anatomies may not have regular diameters, such as circles, ovals, and/or the like. Accordingly, mounting apparatuses according to some embodiments may be configured to handle different bony anatomy, including irregular shapes and dimensions. Accordingly, mounting apparatuses may have different types and/or shaped arms, for example, as depicted in FIGS. 24A-24G (and variations and derivative forms thereof). In addition, mounting apparatuses according to some embodiments may include projections (for example, projections 2470-2475 and/or variations thereof), and/or the like) of different types and angles. For example, an arm may include projections at an about 90 degree angle (for instance, with respect to a surface of the arm from which projections are extending from), an about 10 degree angle, about 20 degree angle, about 30 degree angle, about 45 degree angle, about 50 degree angle, about 60 degree angle, about 70 degree angle, 80 degree angle, or any value or range between any two of these values (including endpoints). In some embodiments, portions of projections may be at different angles to each other. For example, a portion of projections may be at an about 90 degree angle, while others may be at an about 45 degree angle. In another embodiments, certain projections may be configured for metaphysis and others for diaphysis. Embodiments are not limited in this context.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the certain embodiments or configurations of the disclosure may be combined in alternate embodiments or configurations.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (for example, proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure.

Connection references (for example, engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (for example, primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

What is claimed is:

1. A mechanically-actuated mounting apparatus for mounting a medical aid device to a bone structure of a human body, comprising:
a pair of opposing arms configured to be arranged around the bone structure and operatively coupled to a rack-and-pinion clamping assembly, the rack-and-pinion clamping assembly comprising configured to move the at least one of the pair of opposing arms in one of a clamping direction toward the bone structure or a releasing direction away from the bone structure, the clamping assembly comprising:
a pinion gear operative to engage a rack portion of the at least one of the pair of opposing arms to:
rotate in a tensioning direction to cause the at least one of the pair of opposing arms to move in the clamping direction to affix the mechanically-actuated mounting apparatus to the bone structure, and
rotate in a relaxing direction to cause the at least one of the pair of opposing arms to move in the releasing direction to release the mechanically-actuated mounting apparatus from the bone structure.

2. The mechanically-actuated mounting apparatus of claim 1, the rack-and-pinion clamping assembly comprising a ratchet pawl configured to prevent rotation of the pinion gear in the relaxing direction.

3. The mechanically-actuated mounting apparatus of claim 2, the rack-and-pinion clamping assembly comprising a biasing element configured to bias ratchet pawl in a direction to prevent rotation of the pinion gear in the relaxing direction.

4. The mechanically-actuated mounting apparatus of claim 3, the rack-and-pinion clamping assembly comprising a release element configured to move into an open position to disengage ratchet pawl from preventing rotation of the pinion gear in the relaxing direction to allow pinion to move in the relaxing direction.

5. The mechanically-actuated mounting apparatus of claim 1, the rack portion comprising a cylindrical rack.

6. The mechanically-actuated mounting apparatus of claim 1, further comprising:
a housing having the rack-and-pinion clamping assembly arranged therein,
the pair of opposing arms comprising a first arm integral to the housing and a second arm having the rack portion.

7. The mechanically-actuated mounting apparatus of claim 1, the rack-and-pinion clamping assembly comprising an anti-rotation hub comprising anti-rotation teeth, the pinion gear comprising ratchet kick teeth operative to engage the anti-rotation teeth to prevent rotation of the pinion gear in the relaxing direction when the anti-rotation hub is in a closed position.

8. The mechanically-actuated mounting apparatus of claim 7, the anti-rotation teeth configured to allow rotation of the pinion gear in the tensioning direction when the anti-rotation hub is in the closed position.

9. The mechanically-actuated mounting apparatus of claim 7, the anti-rotation hub configured to be moved into an open position to disengage anti-rotation teeth from the ratchet kick teeth to allow the pinion gear to rotate in the relaxing direction.

10. The mechanically-actuated mounting apparatus of claim 1, the medical aid device comprising one or more of a tracking array, a sensor, an image capturing device, a video capturing device, a logic device, or a wireless transmitter/receiver device.

11. The mechanically-actuated mounting apparatus of claim 1, the bone structure comprising a portion of a femur.

12. A magnetically-actuated mounting apparatus for mounting a medical aid device to a bone structure of a human body, comprising:
a pair of opposing arms; and
a magnetically-actuated clamping assembly operatively coupled to at least one of the pair of opposing arms, the magnetically-actuated clamping assembly operative to enter an active state responsive to generation of a magnetic clamping force and enter an inactive state responsive to removal of the magnetic clamping force;
wherein, in the active state, the pair of opposing arms is compressed in a clamping direction around the bone structure to rigidly affix the magnetically-actuated mounting apparatus to the bone structure,
wherein, in the inactive state, the pair of opposing arms is operative to move in a releasing direction to release the magnetically-actuated mounting apparatus from the bone structure.

13. The magnetically-actuated mounting apparatus of claim 12, the magnetically-actuated clamping assembly comprising a fixed magnet having a fixed magnetic field and an actuator associated with an actuator magnet having an actuator magnetic field.

14. The magnetically-actuated mounting apparatus of claim 13, the actuator configured to move to an engaged position to align the fixed magnetic field and the actuator magnetic field to generate the magnetic clamping force.

15. The magnetically-actuated mounting apparatus of claim 13, the magnetic clamping force generated responsive to the fixed magnetic field and the actuator magnetic field being aligned and the fixed magnetic field and the actuator magnetic field being within a threshold distance.

16. The magnetically-actuated mounting apparatus of claim 13, the actuator configured to rotate about a transverse axis of the magnetically-actuated clamping assembly to move to the engaged position or the disengaged position.

17. A spring-actuated mounting apparatus configured to mount a medical aid device to a bone structure of a human body, comprising:
a spring-actuated clamping assembly; and
a pair of opposing arms coupled to the spring-actuated clamping assembly, the spring-actuated clamping assembly comprising:
a pin extending through a connection end of each of the pair of opposing arms, each of the pair of opposing arms configured to rotate in one of a clamping direction or a releasing direction about the pin, and
a spring arranged around a portion of the pin, the spring comprising a pair of hooks extending away from a central body of the spring, each of the pair of hooks arranged to engage a portion of one of the pair of arms to bias an engagement end of each of the pair of arms in the clamping direction to affix the spring-actuated mounting apparatus to the bone portion.

18. The spring-actuated mounting assembly of claim 17, the bone portion comprising a femur, the pair of opposing arms comprising an anterior arm and a posterior arm, the anterior arm configured to engage an anterior side of a lesser trochanter region of the femur, and the posterior arm configured to engage a posterior side of the lesser trochanter region of the femur.

19. The spring-actuated mounting assembly of claim 17, at least one of the pair of opposing arms comprising a release attachment configured to receive a release device to place the spring-actuated mounting apparatus in an open position.

20. The spring-actuated mounting assembly of claim 17, the pin comprising an internally-threaded barrel configured to receive a corresponding externally-threaded fastener configured to be threaded into the barrel to form the pin.
